SEQUENCE LISTING (41 sequences)

SEQ ID NO: 1 - DNA, 2412 bp, Homo sapiens
atgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaa

SEQ ID NO: 2 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 3 - protein, 4 aa, Homo sapiens
KDEL

SEQ ID NO: 4 - DNA, 1455 bp, Homo sapiens
atgagactgggaagccctggcctgctgtttctgctgttcagcagcctgagagccgacacccaggaaaaagaagtgcgggccatggtgggaagcgacgtggaactgagctgcgcctgtcctgagggcagcagattcgacctgaacgacgtgtacgtgtactggcagaccagcgagagcaagaccgtcgtgacctaccacatcccccagaacagctccctggaaaacgtggacagccggtacagaaaccgggccctgatgtctcctgccggcatgctgagaggcgacttcagcctgcggctgttcaacgtgaccccccaggacgagcagaaattccactgcctggtgctgagccagagcctgggcttccaggaagtgctgagcgtggaagtgaccctgcacgtggccgccaatttcagcgtgccagtggtgtctgccccccacagcccttctcaggatgagctgaccttcacctgtaccagcatcaacggctaccccagacccaatgtgtactggatcaacaagaccgacaacagcctgctggaccaggccctgcagaacgataccgtgttcctgaacatgcggggcctgtacgacgtggtgtccgtgctgagaatcgccagaacccccagcgtgaacatcggctgctgcatcgagaacgtgctgctgcagcagaacctgaccgtgggcagccagaccggcaacgacatcggcgagagagacaagatcaccgagaaccccgtgtccaccggcgagaagaatgccgccacctctaagtacggccctccctgcccttcttgcccagcccctgaatttctgggcggaccctccgtgtttctgttccccccaaagcccaaggacaccctgatgatcagccggacccccgaagtgacctgcgtggtggtggatgtgtcccaggaagatcccgaggtgcagttcaattggtacgtggacggggtggaagtgcacaacgccaagaccaagcccagagaggaacagttcaacagcacctaccgggtggtgtctgtgctgaccgtgctgcaccaggattggctgagcggcaaagagtacaagtgcaaggtgtccagcaagggcctgcccagcagcatcgaaaagaccatcagcaacgccaccggccagcccagggaaccccaggtgtacacactgccccctagccaggaagagatgaccaagaaccaggtgtccctgacctgtctcgtgaagggcttctacccctccgatatcgccgtggaatgggagagcaacggccagccagagaacaactacaagaccacccccccagtgctggacagcgacggctcattcttcctgtactcccggctgacagtggacaagagcagctggcaggaaggcaacgtgttcagctgcagcgtgatgcacgaagccctgcacaaccactacacccagaagtccctgtctctgtccctgggcaaatga

SEQ ID NO: 5 - protein, 484 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 6 - DNA, 1305 bp, Homo sapiens
atgtctaagtacggccctccctgccctagctgccctgcccctgaatttctgggcggacccagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggattggctgagcggcaaagagtacaagtgcaaggtgtccagcaagggcctgcccagcagcatcgagaaaaccatcagcaacgccaccggccagcccagggaaccccaggtgtacacactgccccctagccaggaagagatgaccaagaaccaggtgtccctgacctgtctcgtgaagggcttctacccctccgatatcgccgtggaatgggagagcaacggccagcctgagaacaactacaagaccacccccccagtgctggacagcgacggctcattcttcctgtacagcagactgaccgtggacaagagcagctggcaggaaggcaacgtgttcagctgcagcgtgatgcacgaggccctgcacaaccactacacccagaagtccctgtctctgagcctgggcaaggcctgtccatgggctgtgtctggcgctagagcctctcctggatctgccgccagccccagactgagagagggacctgagctgagccccgatgatcctgccggactgctggatctgagacagggcatgttcgcccagctggtggcccagaacgtgctgctgatcgatggccccctgagctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatccggctctgtgtctctggctctgcatctgcagcccctgagatctgctgctggcgctgctgctctggccctgacagtggacctgcctcctgcctctagcgaggccagaaacagcgcattcgggtttcaaggcagactgctgcacctgtctgccggccagagactgggagtgcatctgcacacagaggccagagccaggcacgcctggcagctgactcagggcgctacagtgctgggcctgttcagagtgacccccgagattccagccggcctgcctagccccagatccgaatga

SEQ ID NO: 7 - protein, 434 aa, Homo sapiens
MSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 8 - DNA, 1284 bp, Homo sapiens
atgtctaagtacggccctccctgccctagctgccctgcccctgaatttctgggcggacccagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggattggctgagcggcaaagagtacaagtgcaaggtgtccagcaagggcctgcccagcagcatcgagaaaaccatcagcaacgccaccggccagcccagggaaccccaggtgtacacactgccccctagccaggaagagatgaccaagaaccaggtgtccctgacctgtctcgtgaagggcttctacccctccgatatcgccgtggaatgggagagcaacggccagcctgagaacaactacaagaccacccccccagtgctggacagcgacggctcattcttcctgtacagcagactgaccgtggacaagagcagctggcaggaaggcaacgtgttcagctgcagcgtgatgcacgaggccctgcacaaccactacacccagaagtccctgtctctgagcctgggcaagatcgagggccggatggatagagcccagggcgaagcctgcgtgcagttccaggctctgaagggccaggaattcgcccccagccaccagcaggtgtacgcccctctgagagccgacggcgataagcctagagcccacctgacagtcgtgcggcagacccctacccagcacttcaagaatcagttccccgccctgcactgggagcacgaactgggcctggccttcaccaagaacagaatgaactacaccaacaagtttctgctgatccccgagagcggcgactacttcatctacagccaagtgaccttccggggcatgaccagcgagtgcagcgagatcagacaggccggcagacctaacaagcccgacagcatcaccgtcgtgatcaccaaagtgaccgacagctaccccgagcccacccagctgctgatgggcaccaagagcgtgtgcgaagtgggcagcaactggttccagcccatctacctgggcgccatgtttagtctgcaagagggcgacaagctgatggtcaacgtgtccgacatcagcctggtggattacaccaaagaggacaagaccttcttcggcgcctttctgctctga

SEQ ID NO: 9 - protein, 427 aa, Homo sapiens
MSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 10 - DNA, 1107 bp, Homo sapiens
atgtctaagtacggccctccctgccctagctgccctgcccctgaatttctgggcggacccagcgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgaccgtgctgcaccaggattggctgagcggcaaagagtacaagtgcaaggtgtccagcaagggcctgcccagcagcatcgagaaaaccatcagcaacgccaccggccagcccagggaaccccaggtgtacacactgccccctagccaggaagagatgaccaagaaccaggtgtccctgacctgtctcgtgaagggcttctacccctccgatatcgccgtggaatgggagagcaacggccagcctgagaacaactacaagaccacccccccagtgctggacagcgacggctcattcttcctgtacagcagactgaccgtggacaagagcagctggcaggaaggcaacgtgttcagctgcagcgtgatgcacgaggccctgcacaaccactacacccagaagtccctgtctctgagcctgggcaagatcgagggccggatggatcaggtgtcacacagatacccccggatccagagcatcaaagtgcagtttaccgagtacaagaaagagaagggctttatcctgaccagccagaaagaggacgagatcatgaaggtgcagaacaacagcgtgatcatcaactgcgacgggttctacctgatcagcctgaagggctacttcagtcaggaagtgaacatcagcctgcactaccagaaggacgaggaacccctgttccagctgaagaaagtgcggagcgtgaacagcctgatggtggcctctctgacctacaaggacaaggtgtacctgaacgtgaccaccgacaacaccagcctggacgacttccacgtgaacggcggcgagctgatcctgattcaccagaaccccggcgagttctgcgtgctctga

SEQ ID NO: 11 - protein, 368 aa, Homo sapiens
MSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 12 - protein, 1588 aa, Homo sapiens
TCCCAAGTAGCTGGGACTACAGGAGCCCACCACCACCCCCGGCTAATTTTTTGTATTTTTAGTAGAGACGGGGTTTCACCGTGTTAGCCAAGATGGTCTTGATCACCTGACCTCGTGATCCACCCGCCTTGGCCTCCCAAAGTGCTGGGATTACAGGCATGAGCCACCGCGCCCGGCCTCCATTCAAGTCTTTATTGAATATCTGCTATGTTCTACACACTGTTCTAGGTGCTGGGGATGCAACAGGGGACAAAATAGGCAAAATCCCTGTCCTTTTGGGGTTGACATTCTAGTGACTCTTCATGTAGTCTAGAAGAAGCTCAGTGAATAGTGTCTGTGGTTGTTACCAGGGACACAATGACAGGAACATTCTTGGGTAGAGTGAGAGGCCTGGGGAGGGAAGGGTCTCTAGGATGGAGCAGATGCTGGGCAGTCTTAGGGAGCCCCTCCTGGCATGCACCCCCTCATCCCTCAGGCCACCCCCGTCCCTTGCAGGAGCACCCTGGGGAGCTGTCCAGAGCGCTGTGCCGCTGTCTGTGGCTGGAGGCAGAGTAGGTGGTGTGCTGGGAATGCGAGTGGGAGAACTGGGATGGACCGAGGGGAGGCGGGTGAGGAGGGGGGCAACCACCCAACACCCACCAGCTGCTTTCAGTGTTCTGGGTCCAGGTGCTCCTGGCTGGCCTTGTGGTCCCCCTCCTGCTTGGGGCCACCCTGACCTACACATACCGCCACTGCTGGCCTCACAAGCCCCTGGTTACTGCAGATGAAGCTGGGATGGAGGCTCTGACCCCACCACCGGCCACCCATCTGTCACCCTTGGACAGCGCCCACACCCTTCTAGCACCTCCTGACAGCAGTGAGAAGATCTGCACCGTCCAGTTGGTGGGTAACAGCTGGACCCCTGGCTACCCCGAGACCCAGGAGGCGCTCTGCCCGCAGGTGACATGGTCCTGGGACCAGTTGCCCAGCAGAGCTCTTGGCCCCGCTGCTGCGCCCACACTCTCGCCAGAGTCCCCAGCCGGCTCGCCAGCCATGATGCTGCAGCCGGGCCCGCAGCTCTACGACGTGATGGACGCGGTCCCAGCGCGGCGCTGGAAGGAGTTCGTGCGCACGCTGGGGCTGCGCGAGGCAGAGATCGAAGCCGTGGAGGTGGAGATCGGCCGCTTCCGAGACCAGCAGTACGAGATGCTCAAGCGCTGGCGCCAGCAGCAGCCCGCGGGCCTCGGAGCCGTTTACGCGGCCCTGGAGCGCATGGGGCTGGACGGCTGCGTGGAAGACTTGCGCAGCCGCCTGCAGCGCGGCCCGTGACACGGCGCCCACTTGCCACCTAGGCGCTCTGGTGGCCCTTGCAGAAGCCCTAAGTACGGTTACTTATGCGTGTAGACATTTTATGTCACTTATTAAGCCGCTGGCACGGCCCTGCGTAGCAGCACCAGCCGGCCCCACCCCTGCTCGCCCCTATCGCTCCAGCCAAGGCGAAGAAGCACGAACGAATGTCGAGAGGGGGTGAAGACATTTCTCAACTTCTCGGCCGGAGTTTGGCTGAGATCGCGGTATTAAATCTGTGAAAGAAAACAAAACAAAACAA

SEQ ID NO: 13 - protein, 426 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTPPPSLAGAPWGAVQSAVPLSVAGGRVGVFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 14 - protein, 5 aa, Artificial Sequence
  Synthetic Sequence
GGGGS

SEQ ID NO: 15 - protein, 5 aa, Artificial Sequence
  Synthetic Sequence
GGGGS

SEQ ID NO: 16 - protein, 8 aa, Artificial Sequence
  Synthetic Sequence
GGGGGGGG

SEQ ID NO: 17 - protein, 6 aa, Artificial Sequence
  Synthetic Sequence
GGGGGG

SEQ ID NO: 18 - protein, 5 aa, Artificial Sequence
  Synthetic Sequence
EAAAK

SEQ ID NO: 19 - protein, 12 aa, Artificial Sequence
  Synthetic Sequence
AEAAAKEAAAKA

SEQ ID NO: 20 - protein, 12 aa, Artificial Sequence
  Synthetic Sequence
AEAAAKEAAAKA

SEQ ID NO: 21 - protein, 46 aa, Artificial Sequence
  Synthetic Sequence
AEAAAKEAAAKEAAAKEAAAKALEAEAAAKEAAAKEAAAKEAAAKA

SEQ ID NO: 22 - protein, 5 aa, Artificial Sequence
  Synthetic Sequence
PAPAP

SEQ ID NO: 23 - protein, 18 aa, Artificial Sequence
  Synthetic Sequence
KESGSVSSEQLAQFRSLD

SEQ ID NO: 24 - protein, 14 aa, Artificial Sequence
  Synthetic Sequence
EGKSSGSGSESKST

SEQ ID NO: 25 - protein, 12 aa, Artificial Sequence
  Synthetic Sequence
GSAGSAAGSGEF

SEQ ID NO: 26 - DNA, 852 bp, Homo sapiens
atggagcctcctggagactgggggcctcctccctggagatccacccccaaaaccgacgtcttgaggctggtgctgtatctcaccttcctgggagccccctgctacgccccagctctgccgtcctgcaaggaggacgagtacccagtgggctccgagtgctgccccaagtgcagtccaggttatcgtgtgaaggaggcctgcggggagctgacgggcacagtgtgtgaaccctgccctccaggcacctacattgcccacctcaatggcctaagcaagtgtctgcagtgccaaatgtgtgacccagccatgggcctgcgcgcgagccggaactgctccaggacagagaacgccgtgtgtggctgcagcccaggccacttctgcatcgtccaggacggggaccactgcgccgcgtgccgcgcttacgccacctccagcccgggccagagggtgcagaagggaggcaccgagagtcaggacaccctgtgtcagaactgccccccggggaccttctctcccaatgggaccctggaggaatgtcagcaccagaccaagtgcagctggctggtgacgaaggccggagctgggaccagcagctcccactgggtatggtggtttctctcagggagcctcgtcatcgtcattgtttgctccacagttggcctaatcatatgtgtgaaaagaagaaagccaaggggtgatgtagtcaaggtgatcgtctccgtccagcggaaaagacaggaggcagaaggtgaggccacagtcattgaggccctgcaggcccctccggacgtcaccacggtggccgtggaggagacaataccctcattcacggggaggagcccaaaccattaa

SEQ ID NO: 27 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 28 - DNA, 4900 bp, Homo sapiens
taaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaagaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatggcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaaatggagataatggttacaaatgtctcttcctatagtataatctccataagggcatggcccaagtctgtctttgactctgcctatccctgacatttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccagatcgaaaatactgtactttggttgatttttaagtgggcttccattccatggatttaatcagtcccaagaagatcaaactcagcagtacttgggtgctgaagaactgttggatttaccctggcacgtgtgccacttgccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactactatatgtctgctttaaatttgtactttaatattgtcttttggtattaagaaagatatgctttcagaatagatatgcttcgctttggcaaggaatttggatagaacttgctatttaaaagaggtgtggggtaaatccttgtataaatctccagtttagccttttttgaaaaagctagactttcaaatactaatttcacttcaagcagggtacgtttctggtttgtttgcttgacttcagtcacaatttcttatcagaccaatggctgacctctttgagatgtcaggctaggcttacctatgtgttctgtgtcatgtgaatgctgagaagtttgacagagatccaacttcagccttgaccccatcagtccctcgggttaactaactgagccaccggtcctcatggctattttaatgagggtattgatggttaaatgcatgtctgatcccttatcccagccatttgcactgccagctgggaactataccagacctggatactgatcccaaagtgttaaattcaactacatgctggagattagagatggtgccaataaaggacccagaaccaggatcttgattgctatagacttattaataatccaggtcaaagagagtgacacacactctctcaagacctggggtgagggagtctgtgttatctgcaaggccatttgaggctcagaaagtctctctttcctatagatatatgcatactttctgacatataggaatgtatcaggaatactcaaccatcacaggcatgttcctacctcagggcctttacatgtcctgtttactctgtctagaatgtccttctgtagatgacctggcttgcctcgtcacccttcaggtccttgctcaagtgtcatcttctcccctagttaaactaccccacaccctgtctgctttccttgcttatttttctccatagcattttaccatctcttacattagacatttttcttatttatttgtagtttataagcttcatgaggcaagtaactttgctttgtttcttgctgtatctccagtgcccagagcagtgcctggtatataataaatatttattgactgagtgaaaaaaaaaaaaaaaaa

SEQ ID NO: 29 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 30 - DNA, 1906 bp, Homo sapiens
ccaagtcacatgattcaggattcagggggagaatccttcttggaacagagatgggcccagaactgaatcagatgaagagagataaggtgtgatgtggggaagactatataaagaatggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagagctccattcaagaagtcatgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactgaacagtttctcttggccttcaggaagaaagcgcctctctaccatacagtatttcatccctccaaacacttgggcaaaaagaaaactttagaccaagacaaactacacagggtattaaatagtatacttctccttctgtctcttggaaagatacagctccagggttaaaaagagagtttttagtgaagtatctttcagatagcaggcagggaagcaatgtagtgtggtgggcagagccccacacagaatcagaagggatgaatggatgtcccagcccaaccactaattcactgtatggtcttgatctatttcttctgttttgagagcctccagttaaaatggggcttcagtaccagagcagctagcaactctgccctaatgggaaatgaaggggagctgggtgtgagtgtttacactgtgcccttcacgggatacttcttttatctgcagatggcctaatgcttagttgtccaagtcgcgatcaaggactctctcacacaggaaacttccctatactggcagatacacttgtgactgaaccatgcccagtttatgcctgtctgactgtcactctggcactaggaggctgatcttgtactccatatgaccccacccctaggaacccccagggaaaaccaggctcggacagccccctgttcctgagatggaaagcacaaatttaatacaccaccacaatggaaaacaagttcaaagacttttacttacagatcctggacagaaagggcataatgagtctgaagggcagtcctccttctccaggttacatgaggcaggaataagaagtcagacagagacagcaagacagttaacaacgtaggtaaagaaatagggtgtggtcactctcaattcactggcaaatgcctgaatggtctgtctgaaggaagcaacagagaagtggggaatccagtctgctaggcaggaaagatgcctctaagttcttgtctctggccagaggtgtggtatagaaccagaaacccatatcaagggtgactaagcccggcttccggtatgagaaattaaacttgtatacaaaatggttgccaaggcaacataaaattataagaattc

SEQ ID NO: 31 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 32 - DNA, 1629 bp, Homo sapiens
tttcctgggcggggccaaggctggggcaggggagtcagcagaggcctcgctcgggcgcccagtggtcctgccgcctggtctcacctcgctatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctggggg

SEQ ID NO: 33 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 34 - DNA, 913 bp, Homo sapiens
ccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctgaccactgctgctgattagggttttttaaattttattttattttatttaagttcaagagaaaaagtgtacacacaggggccacccggggttggggtgggagtgtggtggggggtagtggtggcaggacaagagaaggcattgagctttttctttcattttcctattaaaaaatacaaaaatca

SEQ ID NO: 35 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 36 - DNA, 723 bp, Homo sapiens
atggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggccgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtgtga

SEQ ID NO: 37 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 38 - protein, 9 aa, Artificial Sequence
  Synthetic Sequence
EGSRNQDWL

SEQ ID NO: 39 - protein, 8 aa, Artificial Sequence
  Synthetic Sequence
TWHRYHLL

SEQ ID NO: 40 - protein, 9 aa, Artificial Sequence
  Synthetic Sequence
SVYDFFVWL

SEQ ID NO: 41 - protein, 8 aa, Artificial Sequence
  Synthetic Sequence
SIINFEKL

(19) United States
(12) Patent Application Publication
(10) Pub. No.: US 2021/0346486 A1
(43) Pub. Date: Nov. 11, 2021

(54) COMBINATION CELL-BASED THERAPIES

(71) Applicant: Heat Biologics, Inc., Morrisville, NC (US)

(72) Inventor: Jeff HUTCHINS, Morrisville, NC (US)

(21) Appl. No.: 17/277,871

(22) PCT Filed: Oct. 1, 2019
(86) PCT No.: PCT/US2019/053925

§ 371 Date: Mar. 19, 2021

Related U.S. Application Data
(60) Provisional application No. 62/807,783, filed on Feb. 20, 2019.
(60) Provisional application No. 62/739,814, filed on Oct. 1, 2018.

(51) Int. Cl. A61K 39/00; C07K 16/28; C07K 14/705; A61P 35/00

(52) U.S. Cl. CPC A61K 39/001176; C07K 16/2818; A61K 2039/505; A61P 35/00; A61K 2039/5152; C07K 14/70575

(57) ABSTRACT

The present disclosure provides methods of treatment with cells having a vaccine (e.g., gp96-Ig) and cells having a T-cell co-stimulatory molecule.

### CROSS-REFERENCE TO RELATED APPLICATIONS

This application claims priority to and the benefit of U.S. Provisional Patent Application No. 62/739,814, filed on Oct. 1, 2018, and U.S. Provisional Patent Application No. 62/807,783, filed on Feb. 20, 2019, the entire contents of which are herein incorporated by reference herein in their entireties.

### FIELD OF THE DISCLOSURE

The disclosure is directed to methods of treatment with cells having a vaccine (e.g., gp96-Ig) and cells having T-cell co-stimulatory molecules.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: HTB-030_ST25.txt; date recorded: Sep. 30, 2019; file size: 79.2 KB).

### BACKGROUND

Cancer is characterized by a progressive acquisition of genetic mutations that lead to intrinsic dysregulation of cell growth and death. Once a cell has acquired enough mutations, typically thought to be at least six, it no longer is responsive to intrinsic or extrinsic signals that would restrain its growth or trigger apoptosis. As tumors arise from host cells, the body's immune system is initially tolerant to those cells. A cell that acquires an immunogenic mutation, can be sought out and destroyed by the host immune system in a process known as immunosurveillance. Immune checkpoint therapy, which targets regulatory pathways in T cells to enhance anti-tumor immune responses, has led to important clinical advances and provides a new defense against cancer. Moreover, vaccines may contribute to this defense by also enhancing anti-tumor immune responses. Accordingly, it is possible that combination therapies including combinations or sub-combinations of one or more checkpoint inhibitors, one or more vaccines, and one or more T cell costimulatory molecules may expand the base of cancer patients that can benefit from immunotherapy.

### SUMMARY

Immunotherapies aimed at including combinations of one or more vaccines, one or more T cell costimulatory molecules, and one or more checkpoint inhibitors may expand the base of cancer patients that can benefit from such therapies. Vaccines may contribute to this response by increasing both the frequency of tumor-antigen specific CD8+ T cells and also the number of tumor antigens recognized by those CD8+ T cells. T cell costimulatory molecules may enhance the response by further increasing the frequency and/or enhancing the activation of tumor antigen-specific T cells, and also by increasing the expression of tumor-killing effector molecules by CD8+ T cells. When used in combination with checkpoint inhibitors, it may be possible to generate a broad range of highly activated CD8+ T cells that will be able to infiltrate tumors and will not be inhibited by various checkpoint pathways once infiltration has occurred. This present disclosure is based, at least in part, on the discovery that a combination of a vaccination, e.g., gp96-Ig vaccination, and T cell costimulation with one or more agonists of OX40, ICOS, 4-1BB, TNFRSF25, CD40, CD27, and/or GITR, among others, provides a synergistic anti-tumor benefit. Pre-clinical models have evaluated independent compositions of gp96-Ig vaccines combined with agonistic antibodies targeting OX40, ICOS, 4-1BB, and TNFRSF25, and demonstrated variable effects on mechanistic and anti-tumor complementarity. The methods described herein provide a first cell comprising an expression vector comprising, a nucleotide sequence that encodes a secretable vaccine protein, (e.g., gp96-Ig) expression vector, wherein the patient is undergoing a treatment with a second cell comprising an expression vector comprising a nucleotide sequence that encodes a T cell costimulatory fusion protein, including, without limitation, fusion proteins such as ICOSL-Ig, 4-1BBL-Ig, TL1A-Ig, OX40L-Ig, CD40L-Ig, CD70-Ig, or GITRL-Ig to provide T cell costimulation.

In some embodiments, the methods described herein secrete fusion proteins that work synergistically. The effect of a locally secreted T-cell costimulatory fusion protein (i.e., OX40L-Ig), inter alia, performs differently than a systemic administration in combination with the secretable vaccine protein (e.g., gp96-Ig). Not wishing to be bound by theory, the effects of a cell secreting a vaccine protein alone (e.g., gp96-Ig) and in combination with escalating doses of a cell secreting a T-cell costimulatory fusion protein (i.e., OX40L-Ig), performs differently when compared to the vaccine protein (e.g., gp96-Ig) and escalating doses of a systemic OX40 agonist antibody.

In some embodiments, the amount of a secretable vaccine protein (e.g., gp96-Ig) secretion is higher than the expression of a T cell costimulatory fusion protein, (e.g., OX40L-Ig). In some embodiments, the ratio of a vaccine protein (e.g., gp96-Ig) secretion to the expression of a T cell costimulatory fusion protein (e.g., OX40L-Ig) is about 1:10, 1:25, 1:50, 1:100, 1:200, 1:300, 1:400, 1:500, 1:600, 1:700, 1:800, 1:900 or 1:1000, (inclusive of all endpoints).

In some embodiments, the amount of a vaccine protein (e.g., gp96-Ig) secretion is lower than the expression of a T cell costimulatory fusion protein (e.g., OX40L-Ig). In some embodiments, the ratio of a vaccine protein (e.g., gp96-Ig) secretion to the expression of a T cell costimulatory fusion protein (e.g., OX40L-Ig) is about 10:1, 25:1, 50:1, 100:1, 200:1, 300:1, 400:1, 500:1, 600:1, 700:1, 800:1, 900:1, or 1000:1, (inclusive of all endpoints).

In some embodiments, the amount of the expression of a T cell costimulatory fusion protein (e.g., OX40L-Ig) is higher than the secretion of a vaccine protein (e.g., gp96-Ig). In some embodiments, the ratio of the expression of a T cell costimulatory fusion protein (e.g., OX40L-Ig) to the secretion of a vaccine protein (e.g., gp96-Ig) is about 1:10, 1:25, 1:50, 1:100, 1:200, 1:300, 1:400, 1:500, 1:600, 1:700, 1:800, 1:900, or 1:1000, (inclusive of all endpoints).

In some embodiments, the amount of the expression of a T cell costimulatory fusion protein (e.g., OX40L-Ig) is lower than the secretion of a vaccine protein (e.g., gp96-Ig). In some embodiments, the ratio of the expression of a T cell costimulatory fusion protein (e.g., OX40L-Ig) to the secretion of a vaccine protein (e.g., gp96-Ig) is about 10:1, 25:1, 50:1, 100:1, 200:1, 300:1, 400:1, 500:1, 600:1, 700:1, 800:1, 900:1, or 1000:1, (inclusive of all endpoints).

In some embodiments, the amount of a secretable vaccine protein (e.g., gp96-Ig) secretion is about the same as the expression of a T cell costimulatory fusion protein, (e.g., OX40L-Ig). In some embodiments, the ratio of a vaccine protein (e.g., gp96-Ig) secretion to the expression of a T cell costimulatory fusion protein (e.g., OX40L-Ig) is about 1:1. In some embodiments, the ratio of a vaccine protein (e.g., gp96-Ig) secretion to the expression of a T cell costimulatory fusion protein, such as OX40L-Ig, is about 1:1.3.

In some embodiments, the amount of a secretable vaccine protein (e.g., gp96-Ig) expression is about the same as the expression of a T cell costimulatory fusion protein, (e.g., OX40L-Ig). In some embodiments, the ratio of a vaccine protein (e.g., gp96-Ig) expression to the expression of a T cell costimulatory fusion protein (e.g., OX40L-Ig) is about 1:1. In some embodiments, the ratio of a vaccine protein (e.g., gp96-Ig) expression to the expression of a T cell costimulatory fusion protein, such as OX40L-Ig, is about 1:1.3.

In some embodiments, the amount of the expression of a T cell costimulatory fusion protein (e.g., OX40L-Ig) is about the same as the secretion of a vaccine protein (e.g., gp96-Ig). In some embodiments, the ratio of the expression of a T cell costimulatory fusion protein (e.g., OX40L-Ig) to the secretion of a vaccine protein (e.g., gp96-Ig) is about 1:1.

In some embodiments, the number of cells secreting a gp96-Ig is higher than the number of cells secreting a OX40L-Ig. In some embodiments, the ratio of the number of cells secreting a gp96-Ig to the number of cells secreting a OX40L-Ig is about 1:0.01, about 1:0.1, about 1:1, about 1:10, about 1:25, about 1:50, about 1:100, about 1:200, about 1:300, about 1:400, about 1:500, about 1:600, about 1:700, about 1:800, about 1:900 or about 1:1000 (inclusive of all endpoints).

In some embodiments, the number of cells secreting a gp96-Ig is lower than the number of cells secreting a OX40L-Ig. In some embodiments, the ratio of the number of cells secreting a gp96-Ig to the number of cells secreting a OX40L-Ig is about 0.01:1, about 0.1:1, about 1:1, about 1:1.3, about 10:1, about 25:1, about 50:1, about 100:1, about 200:1, about 300:1, about 400:1, about 500:1, about 600:1, about 700:1, about 800:1, about 900:1, or about 1000:1 (inclusive of all endpoints).

In some embodiments, the expression of a gp96-Ig is higher than the expression of a OX40L-Ig. In some embodiments, the ratio of the expression of the gp96-Ig to the expression of the OX40L-Ig is about 1:0.01, about 1:0.1, about 1:1, about 1:10, about 1:25, about 1:50, about 1:100, about 1:200, about 1:300, about 1:400, about 1:500, about 1:600, about 1:700, about 1:800, about 1:900 or about 1:1000 (inclusive of all endpoints).

In some embodiments, the expression of a gp96-Ig is lower than the expression of a OX40L-Ig. In some embodiments, the ratio of the expression of a gp96-Ig to the expression of a OX40L-Ig is about 0.01:1, about 0.1:1, about 1:1, about 1:3, about 10:1, about 25:1, about 50:1, about 100:1, about 200:1, about 300:1, about 400:1, about 500:1, about 600:1, about 700:1, about 800:1, about 900:1, or about 1000:1 (inclusive of all endpoints).

In some embodiments, an inducible promoter can be used for inducing the expression of the vaccine protein (e.g., gp96-Ig). In some embodiments, the gp96-Ig is under a strong inducible promoter. In some embodiments, the gp96-Ig is under an intermediate inducible promoter. In some embodiments, the gp96-Ig is under a weak inducible promoter.

In some embodiments, an inducible promoter can be used for inducing the expression T cell costimulatory fusion (e.g., OX40L-Ig). In some embodiments, the OX40L-Ig is under a strong inducible promoter. In some embodiments, the OX40L-Ig is under an intermediate inducible promoter. In some embodiments, the OX40L-Ig is under a weak inducible promoter.

In some embodiments, the vaccine protein (e.g., gp96-Ig) and/or T cell costimulatory fusion protein (e.g., OX40L-Ig) are expressed in host cells (e.g., mammalian cells). In some embodiments, expression and/or secretion of the gp96-Ig and/or OX40L-Ig can be readily detected and quantified by techniques known in the art, such as, in vitro cell culturing methods or protein detection assays. In some embodiments, the protein detection assays include enzyme-linked immunosorbent assay (ELISA), immunoprecipitation, and fluorescence based methods.

In some embodiments, the amount of secreted gp96-Ig by a cell is higher than the amount of secreted OX40L-Ig by a cell. In some embodiments, the ratio of the secreted gp96-Ig by a cell to the secreted OX40L-Ig by a cell is about 1:0.01, about 1:0.1, about 1:1, about 1:10, about 1:25, about 1:50, about 1:100, about 1:200, about 1:300, about 1:400, about 1:500, about 1:600, about 1:700, about 1:800, about 1:900 or about 1:1000 (inclusive of all endpoints).

In some embodiments, the amount of secreted gp96-Ig by a cell is lower than the amount of secreted OX40L-Ig by a cell. In some embodiments, the ratio of secreted gp96-Ig by a cell to secreted OX40L-Ig by a cell is about 0.01:1, about 0.1:1, about 1:1, about 1:1.3, about 10:1, about 25:1, about 50:1, about 100:1, about 200:1, about 300:1, about 400:1, about 500:1, about 600:1, about 700:1, about 800:1, about 900:1, or about 1000:1 (inclusive of all endpoints).

In one aspect, the disclosure provides a method for treating a patient comprising administering to the patient an effective amount of a first cell comprising an expression vector comprising, a nucleotide sequence that encodes a secretable vaccine protein, wherein the patient is undergoing a treatment with a second cell comprising an expression vector comprising a nucleotide sequence that encodes a T cell costimulatory fusion protein and wherein the T cell costimulatory fusion protein enhances activation of antigen-specific T cells when administered to the subject.

In one aspect, the disclosure provides a method for treating a patient comprising administering to the patient an effective amount of a second cell comprising an expression vector comprising a nucleotide sequence that encodes a T cell costimulatory fusion protein, wherein the T cell costimulatory fusion protein enhances activation of antigen-specific T cells when administered to the subject, and wherein the patient is undergoing a treatment with a first cell comprising an expression vector comprising, a nucleotide sequence that encodes a secretable vaccine protein.

In one aspect, the disclosure provides a method for treating a patient comprising administering to the patient an effective amount of (a) a first cell comprising an expression vector comprising a nucleotide sequence that encodes a T cell costimulatory fusion protein and (b) a second cell comprising an expression vector comprising a nucleotide sequence that encodes a T cell costimulatory fusion protein and wherein the T cell costimulatory fusion protein enhances activation of antigen-specific T cells when administered to the subject.

In some embodiments, the secretable vaccine protein is a secretable gp96-Ig fusion protein which optionally lacks the gp96 KDEL (SEQ ID NO:3) sequence. In some embodiments, the Ig tag in the gp96-Ig fusion protein comprises the Fc region of human IgG1, IgG2, IgG3, IgG4, IgM, IgA, or IgE.

In some embodiments, the T cell costimulatory fusion protein is OX40L-Ig, or a portion thereof that binds to OX40. In some embodiments, the T cell costimulatory fusion protein is ICOSL-Ig, or a portion thereof that binds to ICOS. In some embodiments, the T cell costimulatory fusion protein is 4-1BBL-Ig, or a portion thereof that binds to 4-1BBR. In some embodiments, the T cell costimulatory fusion protein is TL1A-Ig, or a portion thereof that binds to TNFRSF25. In some embodiments, the T cell costimulatory fusion protein is GITRL-Ig, or a portion thereof that binds to GITR. In some embodiments, the T cell costimulatory fusion protein is CD40L-Ig, or a portion thereof that binds to CD40. In some embodiments, the T cell costimulatory fusion protein is CD70-Ig, or a portion thereof that binds to CD27. In some embodiments, the Ig tag in the T cell costimulatory fusion protein comprises the Fc region of human IgG1, IgG2, IgG3, IgG4, IgM, IgA, or IgE.

In some embodiments, the expression vector is incorporated into a virus or virus-like particle. In some embodiments, the expression vector is incorporated into a human tumor cell. In some embodiments, the patient is a human cancer patient. In some embodiments, administration to the human patient increases the activation or proliferation of tumor antigen specific T cells in the patient.

In some embodiments, the activation or proliferation of tumor antigen specific T cells in the patient is increased by at least 25 percent as compared to the level of activation or proliferation of tumor antigen specific T cells in the patient prior to the administration.

In some embodiments, administration is in combination with an agent that inhibits immunosuppressive molecules produced by tumor cells. In some embodiments, the agent is an antibody against PD-1. In some embodiments, the antibody against PD-1 is selected from Nivolumab, Pembrolizumab, Pidilizumab, Cemiplimab, AGEN2034, AMP-224, AMP-514, PDR001.

In some embodiments, the patient is a human with an acute or chronic infection. In some embodiments, the acute or chronic infection is an infection by hepatitis C virus, hepatitis B virus, human immunodeficiency virus, or malaria.

In some embodiments, administration to the human patient stimulates the activation or proliferation of pathogenic antigen specific T cells.

In some embodiments, the T cell costimulatory molecule enhances the activation of antigen-specific T cells in the subject to a greater level than gp96-Ig vaccination alone.

### BRIEF DESCRIPTION OF THE DRAWINGS

FIG. 1 is a plasmid vector map for pcDNA3.4 OX40L-Ig.

FIG. 2 is a graph showing the activation of human OX40 receptor in Jurkat cells by mouse and human OX40L.

FIG. 3 is an image showing mouse HS-110 (B16F10-OVA-gp96) and mouse HS-130 (B16F10-OVA-OX40L) immunization over a dose-range to correlate CD8+ T-cell expansion to tumor growth delay.

FIG. 4 shows flow cytometry diagrams, dot plots and gating strategy for Day 7 after a primary vaccination (peripheral blood). Recipient mice were injected with a mouse mHS-110 at a constant dose of 1 million cells (290 ng of gp96-Ig) with different ratios of mHS-130 (0.1, 0.3, 1, 3, 10). A ratio of 1 to 1 is 290 ng of gp96 (1 million mHS-110 cells) to 290 ng of OX40L. FIG. 4 shows a flow cytometry gating strategy using FlowJo Version 10 (2018), by gating in the blood on singlets and CD3+ T cells, then CD8+ OT-1 GFP+ T cells. Sample analysis was taken on day 7 and numbers in representative dot plots indicate percentages of CD8+OT-1 GFP+ positive cells within the gated population. Plots show an individual, representative mouse that illustrates peak expansion for the day selected.

FIG. 5 shows flow cytometry diagrams, dot plots and gating strategy for Day 21 after a boost vaccination (peripheral blood). Recipient mice were injected with a mouse mHS-110 at a constant dose of 1 million cells (290 ng of gp96-Ig) with different ratios of mHS-130 (0.1, 0.3, 1, 3, 10). A ratio of 1 to 1 is 290 ng of gp96 (1 million mHS-110 cells) to 290 ng of OX40L. FIG. 5 shows a flow cytometry gating strategy using FlowJo Version 10 (2018), by gating in the blood on singlets and CD3+ T cells, then CD8+OT-1 GFP+ T cells. Sample analysis was taken on day 21 after a boost immunization that was given on day 14, and numbers in the representative dot plots indicate percentages of CD8+OT-1 GFP+ positive cells within the gated population. Plots show an individual, representative mouse that illustrates peak expansion for the day selected.

FIG. 6A and FIG. 6B are graphs showing the percent of OT-1 CD8+ T-cells after primary and secondary vaccination with a set dose mHS-110 with different ratios of mHS-130 before and after tumor challenge (peripheral blood). Recipient mice were injected with a mHS-110 at a constant dose of 1 million cells (290 ng of gp96-Ig) with different ratios of mHS130. After vaccination, OT-1 GFP+CD8+ T cells were analyzed in the blood on days 0-53 days post-vaccination. Then mice were boosted on day 14 with the same ratios of mHS110 and mHS130 as in the primary phase, and OT-1 GFP+CD8+ T cells were analyzed in the blood on days 17, 19, 21, 24, 28, 33, 38, 41 days post-challenge. Data represent mean total numbers±SEM from n=5 mice. *p<0.05 **p<0.01 (mHS-110 Only versus different ratios of mHS-130). (FIG. 6A): Line graph without overlay; (FIG. 6B): Line graph with mouse outliers removed out to day 41 only.

FIG. 7A, FIG. 7B, FIG. 7C, FIG. 7D, and FIG. 7E are graphs showing the day-54, End-of-Study, endogenous response to vaccination. FIG. 7A is a graph showing an End-of study, day-54, endogenous spleen response to vaccination, percent, gated FSC-H by FSC-A as to live/dead gate for doublets, then gated on CD45 by SSC then CD3+ CD8+ double positive cells. FIG. 7A shows mean±SEM, *p<0.05 compared to mHS-110 via the non-parametric statistical test, Mann-Whitney. FIG. 7B is a flow cytometry diagram and FIG. 7C shows a graph of End-of study, day-54, endogenous spleen response to vaccination and ex vivo stimulation with pg100 peptide for intracellular cytokine staining. Percent shown in graph. Events gated on FSC-H by FSC-A as to live/dead gate for doublets, then gated on CD45 by SSC then IFN-γ CD8+ double positive cells. FIG. 7C is a graph showing mean±SEM, *p<0.05 compared to mHS-110 via the non-parametric statistical test, Mann-Whitney; ‘ns’ denotes p>0.05, not significant. FIG. 7D are graphs showing endogenous spleen immune response measured by IFN-gamma ELISPOT. FIG. 7D shows graphs having mean±SEM IFN-γ spots per million splenocytes, **p<0.01, *p<0.05 compared to mHS-110 via the non-parametric statistical test, Mann-Whitney; ‘ns’ denotes p>0.05, not significant. Far right shows representative ELISPOT well with machine counts. Background (media alone) wells not subtracted from graphed data sets. Positive control wells worked (data not shown). FIG. 7E is a graph showing End-of study, day-54, endogenous response to vaccination, percent, gated FSC-H by FSC-A as to live/dead gate for doublets, then gated on CD45 by SSC then CD3+CD4+ double positive cells. FIG. 7E shows mean±SEM, **p<0.01 compared to mHS-110 via the non-parametric statistical test, Mann-Whitney.

FIG. 8A is a flow cytometry diagram and FIG. 8B is a graph showing CD8+ Tumor Infiltrating Lymphocytes (TILs). FIG. 8A shows graphs of the End-of study, day-54, endogenous TIL response to vaccination, percent, gated FSC-H by FSC-A to as live/dead gate for doublets, then gated on CD45 by SSC then CD3+CD8+ double positive cells. The MACS Miltenyl Biotec tumor dissociation kit was used for this procedure (cat #130-096-730). FIG. 8B is a graph showing mean±SEM, *p<0.05 compared to mHS-110 via the non-parametric statistical test, Mann-Whitney.

FIG. 9 is a graph showing End of study, final tumor mass, in grams, of individual mice. Tumor mass (wet weight) were weighed using a milligram sensitive scale for each animal. FIG. 9 shows mean±SEM. Statistics performed were non-parametric Mann-Whitney, ‘ns’ designates a non-significant (p>0.05) value, *p<0.05; **p<0.01.

FIG. 10A and FIG. 10B shows tumor volumes over time, tumor mean size, and individual plots for individual animals. FIG. 10A is a graph showing mean±SEM for all tumor volumes over time. FIG. 10B is a graph showing means for individual animals for each measured timepoint. Tumor implantation, melanoma B16F10 cells were harvested and resuspended at a concentration of 5×10⁵ cells/100 μl in a volume of 80 μl HBSS and 20 μl Matrigel. C57BL/6 mice were subcutaneously injected with 100 μl of B16F10 cells (5×10⁵ cells/mouse) on the inner abdomen. The tumor size was measured and documented every 3 days with a caliper, starting on day 7, and calculated using the formula (A×B) (A as the largest and B as the smallest diameter of tumor). Tumor growth was documented as standard error mean. To record the survival of the tumor-bearing mice, either natural death or a tumor volume greater than 450 mm² leading to death was counted as death. Each experimental group included five animals. Statistics performed for FIG. 10A was a 2-way ANOVA (shown below FIG. 10A), p<0.05 is considered significantly different vs. mHS-110 group alone.

FIG. 11A and FIG. 11B are graphs showing the percent CD8+OT-1+ T-cells on day 54 (spleen), and the flow plot gating strategy. FIG. 11A is a graph showing the End-of study, day-54, endogenous response to vaccination, percent, gated FSC-H by FSC-A as to live/dead gate for doublets, then gated on CD45 by SSC then GFP-OT-1 CD8+ double positive cells. FIG. 11B is a graph showing mean±SEM, *p<0.05 compared to mHS-110 via the non-parametric statistical test, Mann-Whitney.

FIG. 12A and FIG. 12B are graphs showing the percent CD8+PD-1+ T-cells on day 54 (spleen), and the flow plot gating strategy. FIG. 12A is a graph showing End-of study, day-54, endogenous response to vaccination, percent, gated FSC-H by FSC-A as to live/dead gate for doublets, then gated on CD3 by SSC, then PD-1+CD8+ double positive cells. FIG. 12B is a graph showing mean±SEM, *p<0.05 compared to mHS-110 via the non-parametric statistical test, Mann-Whitney.

FIG. 13 is a non-limiting schematic of the design of the study of gp96-Ig (mHS-110, B16F10-OVA-gp96) to OX40L-Ig (mHS-130, B16F10-OVA-OX40L) dose ratios, to correlate CD8+ T-cell expansion to tumor growth delay.

FIG. 14 are graphs illustrating anti-tumor CD8+OT-I T cell expansion in the peripheral blood with prime and boost Immunization of different ratios and dose combinations of mHS-110 and mHS-130, in the study of FIG. 13. Recipient mice were injected with mHS-110 and mHS-130 at different ratios and doses of gp96-Ig to OX40L-Ig. OT-I GFP+CD8+ T cells were analyzed in the blood on days 0-54 days post-vaccination. Mice were boosted on day 14 with the same ratios of mHS-110 and mHS-130 as in the primary phase, and OT-I GFP+CD8+ T cells were analyzed in the blood days post-challenge. Data represent mean percent±SEM.

FIGS. 15A to 15D illustrate flow cytometry gating strategy and cellular expansion of CD8+OT-I T-cells, over time, with mHS-110/130 immunization in the study of FIG. 13. FIG. 15A are graphs illustrating flow cytometry gating strategy of CD8+OT-I T-cells, over time, for the tested ratios and mHS-110/130 immunization doses. FIG. 15B are bar charts illustrating expansion of CD8+OT-I T-cells on days 7 and 17. FIG. 15C are bar charts illustrating expansion of CD8+OT-I T-cells on days 19, 21, 24, 26, 28, 33, 38, and 41. FIG. 15D are bar charts illustrating expansion of CD8+OT-I T-cells on days 45, 48, and 54. Data represent mean percent±SEM. Statistical analysis was Mann-Whitney, *p<0.05, **p<0.01, ***p<0.001; ‘ns’ denotes p>0.05 or ‘not significant’.

FIGS. 16, 17 and 18 illustrate percent of T-cells in the peripheral blood for SLECs, MPECs, Activated/CD44^{hi} CD8+ endogenous and exogenous (OT-I), T-cells, on day 7 of the study of FIG. 13. FIG. 16 are graphs illustrating flow cytometry gating strategy for MPECs and SLECs. FIG. 17 are bar charts illustrating MPECs and SLEC for endogenous CD8+ T-cells. FIG. 18 bar charts illustrating percent of CD44^{hi} endogenous CD8+ T-cells (% CD8+CD44+ T cells).

FIGS. 19 and 20 are graphs illustrating tumor growth delay/inhibition over time, in the study of FIG. 13. FIG. 19 shows tumor diameter (in mm³) for each of the dose ratio groups, for days 0-28, as mean±SEM grouped tumor diameter growth curves. FIG. 20 are bar charts (left panel) illustrating tumor weights (in grams) as mean±SEM, and scatter plots (right panel) illustrating individual tumors (in grams) as mean±SEM. Statistical analysis performed was Mann-Whitney, *p<0.05, **p<0.01; ‘ns’ denotes p>0.05 or ‘not significant’.

FIG. 21 are bar charts illustrating percent of CD3+CD8+ tetramer-TRP2+ T-cells in the spleen on day 55 of the study of FIG. 13. Graphed values for gated samples are shown, and represent mean percent±SEM. Statistical analysis performed was Mann-Whitney, *p<0.05, **p<0.01; ‘ns’ denotes p>0.05 or ‘not significant’.

FIG. 22 are bar charts illustrating percent of CD3+CD8+eGFP/OT-1+ T-cells in the spleen and blood on day 55 of the study of FIG. 13. CD8+eGFP/OT-1+ T-cells gated for blood and spleen are shown, and represent mean percent±SEM. Statistical analysis performed was Mann-Whitney.

FIG. 23 are bar charts illustrating splenocytes phenotypes on day 55. Data shows percent of CD3+CD4+PD-1+ T cells in the spleen on day 55 of the study of FIG. 13. Data represent mean percent±SEM. Statistical analysis performed was Mann-Whitney, ‘ns’ denotes p>0.05 or ‘not significant’.

FIG. 24 are bar charts illustrating percent of CD3+CD4+CD44/CD62L central memory T-cells in the spleen on day 55 of the study of FIG. 13. Data represent mean percent±SEM. Statistical analysis performed was Mann-Whitney, *p<0.05, **p<0.01.

FIG. 25 are bar charts illustrating tumor infiltrating lymphocytes (TILs) phenotypes. CD8+ TILs (% CD8+CD3+ T-cells) are shown on day 55 of the study of FIG. 13. Data represent mean percent±SEM. Statistical analysis performed was Mann-Whitney, *p<0.05, ‘ns’ denotes p>0.05 or ‘not significant’.

FIG. 26 are bar charts illustrating tumor infiltrating lymphocytes (TILs) phenotypes. CD4+ TILs (% CD4+CD3+ T cells) are shown on day 55 of the study of FIG. 13. Data represent mean percent±SEM from. Statistical analysis performed was Mann-Whitney, *p<0.05; ‘ns’ denotes p>0.05 or ‘not significant’.

### DETAILED DESCRIPTION OF THE DISCLOSURE

The various secretable proteins, i.e., vaccine proteins as described herein, can be used to stimulate an immune response in vivo. For example, secretable heat-shock protein gp96-Ig based allogeneic cellular vaccines can achieve high-frequency polyclonal CD8+ T cell responses to femto-molar concentrations of tumor antigens through antigen cross-priming in vivo (Oizumi et al., J Immunol 2007, 179(4):2310-2317). Multiple immunosuppressive mechanisms elaborated by established tumors can dampen the activity of this vaccine approach, however. In combination immunotherapy for patients with advanced disease, a systematic comparison of PD-1, PD-L1, CTLA-4, and LAG-3 blocking antibodies in mouse models of long-established B16-F10 melanoma demonstrated superior combination between gp96-Ig vaccination and PD-1 blockade as compared to other checkpoints. Synergistic anti-tumor benefits may result from triple combinations of gp96-Ig vaccination, PD-1 blockade, and T cell costimulation using one or of an agonist of OX40 (e.g., an OX40 ligand-Ig (OX40L-Ig) fusion, or a fragment thereof that binds OX40), an agonist of inducible T-cell costimulator (ICOS) (e.g., an ICOS ligand-Ig (ICOSL-Ig) fusion, or a fragment thereof that binds ICOS), an agonist of CD40 (e.g., a CD40L-Ig fusion protein, or fragment thereof), an agonist of CD27 (e.g., a CD70-Ig fusion protein or fragment thereof), an agonist of 4-1BB (e.g., a 4-1BB ligand-Ig (4-1BBL-Ig) fusion, or a fragment thereof that binds 4-1BB), an agonist of TNFRSF25 (e.g., a TL1A-Ig fusion, or a fragment thereof that binds TNFRSF25), or an agonist of glucocorticoid-induced tumor necrosis factor receptor (GITR) (e.g., a GITR ligand-Ig (GITRL-Ig) fusion, or a fragment thereof that binds GITF). Secretion of gp96-Ig and these costimulatory fusion proteins by allogeneic cell lines, enhances activation of antigen-specific CD8+ T cells. Notwithstanding any theory, the effect of a locally secreted T-cell costimulatory fusion protein (i.e., OX40L-Ig), inter alia, performs differently than a systemic administration in combination with the secretable vaccine protein (e.g., gp96-Ig).

### Vaccine Proteins

Vaccine proteins can induce immune responses that find use in the present invention. In some embodiments, the disclosure provides a cell based therapy comprising a first cell comprising an expression vector comprising, a nucleotide sequence that encode a secretable vaccine protein and a second cell comprising an expression vector comprising a nucleotide sequence that encodes a T cell costimulatory fusion protein. Compositions useful in the cell based therapy of the present invention are also provided. In various embodiments, such compositions are utilized in methods of treating subjects to stimulate immune responses in the subject including enhancing the activation of antigen-specific T cells in the subject. The present compositions find use in the treatment of various diseases including cancer.

The heat shock protein (hsp) gp96, localized in the endoplasmic reticulum (ER), serves as a chaperone for peptides on their way to MHC class I and II molecules. Gp96 obtained from tumor cells and used as a vaccine can induce specific tumor immunity, presumably through the transport of tumor-specific peptides to antigen-presenting cells (APCs) (J Immunol 1999, 163(10):5178-5182). For example, gp96-associated peptides are cross-presented to CD8 cells by dendritic cells (DCs).

A vaccination system was developed for antitumor therapy by transfecting a gp96-Ig G1-Fc fusion protein into tumor cells, resulting in secretion of gp96-Ig in complex with chaperoned tumor peptides (see, J Immunother 2008, 31(4):394-401, and references cited therein). Parenteral administration of gp96-Ig secreting tumor cells triggers robust, antigen-specific CD8 cytotoxic T lymphocyte (CTL) expansion, combined with activation of the innate immune system. Tumor-secreted gp96 causes the recruitment of DCs and natural killer (NK) cells to the site of gp96 secretion, and mediates DC activation. Further, the endocytic uptake of gp96 and its chaperoned peptides triggers peptide cross presentation via major MHC class I, as well as strong, cognate CD8 activation independent of CD4 cells.

The cell based therapy provided herein involve a first nucleotide sequence that encodes a gp96-Ig fusion protein. The coding region of human gp96 is 2,412 bases in length (SEQ ID NO:1), and encodes an 803 amino acid protein (SEQ ID NO:2) that includes a 21 amino acid signal peptide at the amino terminus, a potential transmembrane region rich in hydrophobic residues, and an ER retention peptide sequence at the carboxyl terminus (GENBANK® Accession No. X15187; see, Maki et al., Proc Natl Acad Sci USA 1990, 87:5658-5562). The DNA and protein sequences of human gp96 follow:

(SEQ ID NO: 1)
atgagggccctgtgggtgctgggcctctgctgcgtcctgctgaccttcg
ggtcggtcagagctgacgatgaagttgatgtggatggtacagtagaaga
ggatctgggtaaaagtagagaaggatcaaggacggatgatgaagtagta
cagagagaggaagaagctattcagttggatggattaaatgcatcacaaa
taagagaacttagagagaagtcggaaaagtttgccttccaagccgaagt
taacagaatgatgaaacttatcatcaattcattgtataaaaataaagag
attttcctgagagaactgatttcaaatgcttctgatgctttagataaga
taaggctaatatcactgactgatgaaaatgctctttctggaaatgagga
actaacagtcaaaattaagtgtgataaggagaagaacctgctgcatgtc
acagacaccggtgtaggaatgaccagagaagagttggttaaaaaccttg
gtaccatagccaaatctgggacaagcgagtttttaaacaaaatgactga
agcacaggaagatggccagtcaacttctgaattgattggccagtttggt
gtcggtttctattccgccttccttgtagcagataaggttattgtcactt
caaaacacaacaacgatacccagcacatctgggagtctgactccaatga
attttctgtaattgctgacccaagaggaaacactctaggacggggaacg
acaattacccttgtcttaaaagaagaagcatctgattaccttgaattgg
atacaattaaaaatctcgtcaaaaaatattcacagttcataaactttcc
tatttatgtatggagcagcaagactgaaactgttgaggagcccatggag
gaagaagaagcagccaaagaagagaaagaagaatctgatgatgaagctg
cagtagaggaagaagaagaagaaaagaaaccaaagactaaaaaagttga
aaaaactgtctgggactgggaacttatgaatgatatcaaaccaatatgg
cagagaccatcaaaagaagtagaagaagatgaatacaaagctttctaca
aatcattttcaaaggaaagtgatgaccccatggcttatattcactttac
tgctgaaggggaagttaccttcaaatcaattttatttgtacccacatct
gctccacgtggtctgtttgacgaatatggatctaaaaagagcgattaca
ttaagctctatgtgcgccgtgtattcatcacagacgacttccatgatat
gatgcctaaatacctcaattttgtcaagggtgtggtggactcagatgat
ctccccttgaatgtttcccgcgagactcttcagcaacataaactgctta
aggtgattaggaagaagcttgttcgtaaaacgctggacatgatcaagaa
gattgctgatgataaatacaatgatactttttggaaagaatttggtacc
aacatcaagcttggtgtgattgaagaccactcgaatcgaacacgtcttg
ctaaacttcttaggttccagtcttctcatcatccaactgacattactag
cctagaccagtatgtggaaagaatgaaggaaaaacaagacaaaatctac
ttcatggctgggtccagcagaaaagaggctgaatcttctccatttgttg
agcgacttctgaaaaagggctatgaagttatttacctcacagaacctgt
ggatgaatactgtattcaggcccttcccgaatttgatgggaagaggttc
cagaatgttgccaaggaaggagtgaagttcgatgaaagtgagaaaacta
aggagagtcgtgaagcagttgagaaagaatttgagcctctgctgaattg
gatgaaagataaagcccttaaggacaagattgaaaaggctgtggtgtct
cagcgcctgacagaatctccgtgtgctttggtggccagccagtacggat
ggtctggcaacatggagagaatcatgaaagcacaagcgtaccaaacggg
caaggacatctctacaaattactatgcgagtcagaagaaaacatttgaa
attaatcccagacacccgctgatcagagacatgcttcgacgaattaagg
aagatgaagatgataaaacagttttggatcttgctgtggttttgtttga
aacagcaacgcttcggtcagggtatcttttaccagacactaaagcatat
ggagatagaatagaaagaatgcttcgcctcagtttgaacattgaccctg
atgcaaaggtggaagaagagcccgaagaagaacctgaagagacagcaga
agacacaacagaagacacagagcaagacgaagatgaagaaatggatgtg
ggaacagatgaagaagaagaaacagcaaaggaatctacagctgaaaaag
atgaattgtaa
(SEQ ID NO: 2)
MRALWVLGLCCVLLTFGSVRADDEVDVDGTVEEDLGKSREGSRTDDEVV
QREEEAIQLDGLNASQIRELREKSEKFAFQAEVNRMMKLIINSLYKNKE
IFLRELISNASDALDKIRLISLTDENALSGNEELTVKIKCDKEKNLLHV
TDTGVGMTREELVKNLGTIAKSGTSEFLNKMTEAQEDGQSTSELIGQFG
VGFYSAFLVADKVIVISKHNNDTQHIWESDSNEFSVIADPRGNTLGRGT
TITLVLKEEASDYLELDTIKNLVKKYSQFINFPIYVWSSKTETVEEPME
EEEAAKEEKEESDDEAAVEEEEEEKKPKTKKVEKTVWDWELMNDIKPIW
QRPSKEVEEDEYKAFYKSFSKESDDPMAYIHFTAEGEVTFKSILFVPTS
APRGLFDEYGSKKSDYIKLYVRRVFITDDFHDMMPKYLNFVKGVVDSDD
LPLNVSRETLQQHKLLKVIRKKLVRKTLDMIKKIADDKYNDTFWKEFGT
NIKLGVIEDHSNRTRLAKLLRFQSSHHPTDITSLDQYVERMKEKQDKIY
FMAGSSRKEAESSPFVERLLKKGYEVIYLTEPVDEYCIQALPEFDGKRF
QNVAKEGVKFDESEKTKESREAVEKEFEPLLNWMKDKALKDKIEKAWSQ
RLTESPCALVASQYGWSGNMERIMKAQAYQTGKDISTNYYASQKKTFEI
NPRHPLIRDMLRRIKEDEDDKTVLDLAVVLFETATLRSGYLLPDTKAYG
DRIERMLRLSLNIDPDAKVEEEPEEEPEETAEDTTEDTEQDEDEEMDVG
TDEEEETAKESTAEKDEL.

A nucleic acid encoding a gp96-Ig fusion sequence can be produced using the methods described in U.S. Pat. No. 8,685,384, which is incorporated herein by reference in its entirety. In some embodiments, the gp96 portion of a gp96-Ig fusion protein can contain all or a portion of a wild type gp96 sequence (e.g., the human sequence set forth in SEQ ID NO:2). For example, a secretable gp96-Ig fusion protein can include the first 799 amino acids of SEQ ID NO:2, such that it lacks the C-terminal KDEL (SEQ ID NO:3) sequence. Alternatively, the gp96 portion of the fusion protein can have an amino acid sequence that contains one or more substitutions, deletions, or additions as compared to the first 799 amino acids of the wild type gp96 sequence, such that it has at least 90% (e.g., at least 90%, at least 91%, at least 92%, at least 93%, at least 94%, at least 95%, at least 96%, at least 97%, at least 98%, or at least 99%) sequence identity to the wild type polypeptide.

As used throughout this disclosure, the percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:1), or by an articulated length (e.g., 100 consecutive nucleotides or amino acid residues from a sequence set forth in an identified sequence), followed by multiplying the resulting value by 100. For example, a nucleic acid sequence that has 2,200 matches when aligned with the sequence set forth in SEQ ID NO:1 is 91.2 percent identical to the sequence set forth in SEQ ID NO:1 (i.e., 2,000+2,412×100=91.2). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

Thus, in some embodiments, the gp96 portion of nucleic acid encoding a gp96-Ig fusion polypeptide can encode an amino acid sequence that differs from the wild type gp96 polypeptide at one or more amino acid positions, such that it contains one or more conservative substitutions, non-conservative substitutions, splice variants, isoforms, homologues from other species, and polymorphisms.

As defined herein, a “conservative substitution” denotes the replacement of an amino acid residue by another, biologically similar, residue. Typically, biological similarity, as referred to above, reflects substitutions on the wild type sequence with conserved amino acids. For example, conservative amino acid substitutions would be expected to have little or no effect on biological activity, particularly if they represent less than 10% of the total number of residues in the polypeptide or protein. Conservative substitutions may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. Accordingly, conservative substitutions may be effected by exchanging an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Additional examples of conserved amino acid substitutions, include, without limitation, the substitution of one hydrophobic residue for another, such as isoleucine, valine, leucine, or methionine, or the substitution of one polar residue for another, such as the substitution of arginine for lysine, glutamic for aspartic acid, or glutamine for asparagine, and the like. The term “conservative substitution” also includes the use of a substituted amino acid residue in place of an un-substituted parent amino acid residue, provided that antibodies raised to the substituted polypeptide also immunoreact with the un-substituted polypeptide.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g., selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the present fusion proteins by reference to the genetic code, including taking into account codon degeneracy.

The Ig portion (“tag”) of a gp96-Ig fusion protein can contain, for example, a non-variable portion of an immunoglobulin molecule (e.g., an IgG1, IgG2, IgG3, IgG4, IgM, IgA, or IgE molecule). Typically, such portions contain at least functional CH2 and CH3 domains of the constant region of an immunoglobulin heavy chain. Fusions also can be made using the carboxyl terminus of the Fc portion of a constant domain, or a region immediately amino-terminal to the CH1 of the heavy or light chain. The Ig tag can be from a mammalian (e.g., human, mouse, monkey, or rat) immunoglobulin, but human immunoglobulin can be particularly useful when the gp96-Ig fusion is intended for in vivo use for humans.

DNAs encoding immunoglobulin light or heavy chain constant regions are known or readily available from cDNA libraries. See, for example, Adams et al., Biochemistry 1980, 19:2711-2719; Gough et al., Biochemistry 1980 19:2702-2710; Dolby et al., Proc Natl Acad Sci USA 1980, 77:6027-6031; Rice et al., Proc Natl Acad Sci USA 1982, 79:7862-7865; Falkner et al., Nature 1982, 298:286-288; and Morrison et al., Ann Rev Immunol 1984, 2:239-256. Since many immunological reagents and labeling systems are available for the detection of immunoglobulins, gp96-Ig fusion proteins can readily be detected and quantified by a variety of immunological techniques known in the art, such as enzyme-linked immunosorbent assay (ELISA), immunoprecipitation, and fluorescence activated cell sorting (FACS). Similarly, if the peptide tag is an epitope with readily available antibodies, such reagents can be used with the techniques mentioned above to detect, quantitate, and isolate gp96-Ig fusions.

In various embodiments, the gp96-Ig fusion protein and/or the costimulatory molecule fusions, comprises a linker. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In some embodiments, the linker is a synthetic linker such as PEG.

In other embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid. In various embodiments, the linker is substantially comprised of glycine and serine residues (e.g. about 30%, or about 40%, or about 50%, or about 60%, or about 70%, or about 80%, or about 90%, or about 95%, or about 97% glycines and serines).

In various embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

Additional illustrative linkers include, but are not limited to, linkers having the sequence LE, GGGGS (SEQ ID NO:14), (GGGGS)n (n=1-4) (SEQ ID NO: 15), (Gly)8 (SEQ ID NO:16), (Gly)6 (SEQ ID NO:17), (EAAAK)n (n=1-3) (SEQ ID NO: 18), A(EAAAK)nA (n=2-5) (SEQ ID NO: 19), AEAAAKEAAAKA (SEQ ID NO: 20), A(EAAAK)4ALEA(EAAAK)4A (SEQ ID NO: 21), PAPAP (SEQ ID NO: 22), KESGSVSSEQLAQFRSLD (SEQ ID NO: 23), EGKSSGSGSESKST(SEQ ID NO: 24), GSAGSAAGSGEF (SEQ ID NO: 25), and (XP)n, with X designating any amino acid, e.g., Ala, Lys, or Glu.

In various embodiments, the linker may be functional. For example, without limitation, the linker may function to improve the folding and/or stability, improve the expression, improve the pharmacokinetics, and/or improve the bioactivity of the present compositions. In another example, the linker may function to target the compositions to a particular cell type or location.

In some embodiments, a gp96 peptide can be fused to the hinge, CH2 and CH3 domains of murine IgG1 (Bowen et al., J Immunol 1996, 156:442-449). This region of the IgG1 molecule contains three cysteine residues that normally are involved in disulfide bonding with other cysteines in the Ig molecule. Since none of the cysteines are required for the peptide to function as a tag, one or more of these cysteine residues can be substituted by another amino acid residue, such as, for example, serine.

Various leader sequences known in the art also can be used for efficient secretion of gp96-Ig fusion proteins from bacterial and mammalian cells (see, von Heijne, J Mol Biol 1985, 184:99-105). Leader peptides can be selected based on the intended host cell, and may include bacterial, yeast, viral, animal, and mammalian sequences. For example, the herpes virus glycoprotein D leader peptide is suitable for use in a variety of mammalian cells. Another leader peptide for use in mammalian cells can be obtained from the V-J2-C region of the mouse immunoglobulin kappa chain (Bernard et al., Proc Natl Acad Sci USA 1981, 78:5812-5816). DNA sequences encoding peptide tags or leader peptides are known or readily available from libraries or commercial suppliers, and are suitable in the fusion proteins described herein.

Furthermore, in various embodiments, one may substitute the gp96 of the present disclosure with one or more vaccine proteins. For instance, various heat shock proteins are among the vaccine proteins. In various embodiments, the heat shock protein is one or more of a small hsp, hsp40, hsp60, hsp70, hsp90, and hsp110 family member, inclusive of fragments, variants, mutants, derivatives or combinations thereof (Hickey, et al., 1989, Mol. Cell. Biol. 9:2615-2626; Jindal, 1989, Mol. Cell. Biol. 9:2279-2283).

### T-Cell Co-Stimulation

The cell based therapy using the expression vectors provided herein can encode one or more biological response modifiers. In various embodiments, the cell based therapies can encode one or more T cell costimulatory molecules.

In various embodiments, the cell based therapies allow for a robust, antigen-specific CD8 cytotoxic T lymphocyte (CTL) expansion. In various embodiments, the cell based therapies selectively enhance CD8 cytotoxic T lymphocyte (CTL) and do not substantially enhance T cell types that can be pro-tumor, and which include, but are not limited to, Tregs, CD4+ and/or CD8+ T cells expressing one or more checkpoint inhibitory receptors, Th2 cells and Th17 cells. Checkpoint inhibitory receptors refers to receptors (e.g., CTLA-4, B7-H3, B7-H4, TIM-3) expressed on immune cells that prevent or inhibit uncontrolled immune responses. For instance, the present cell based therapies do not substantially enhance FOXP3+ regulatory T cells. In some embodiments, this selective CD8 T cell enhancement is in contrast to the non-specific T cell enhancement observed with a combination therapy of a gp-96 fusion and an antibody against a T cell costimulatory molecule.

For example, the cell based therapies comprise an agonist of OX40 (e.g., an OX40 ligand-Ig (OX40L-Ig) fusion, or a fragment thereof that binds OX40), an agonist of inducible T-cell costimulator (ICOS) (e.g., an ICOS ligand-Ig (ICOSL-Ig) fusion, or a fragment thereof that binds ICOS), an agonist of CD40 (e.g., a CD40L-Ig fusion protein, or fragment thereof), an agonist of CD27 (e.g., a CD70-Ig fusion protein or fragment thereof), or an agonist of 4-1BB (e.g., a 4-1BB ligand-Ig (4-1BBL-Ig) fusion, or a fragment thereof that binds 4-1BB). In some embodiments, the cell based therapies comprise a vector which encodes an agonist of TNFRSF25 (e.g., a TL1A-Ig fusion, or a fragment thereof that binds TNFRSF25), or an agonist of glucocorticoid-induced tumor necrosis factor receptor (GITR) (e.g., a GITR ligand-Ig (GITRL-Ig) fusion, or a fragment thereof that binds GITR), or an agonist of CD40 (e.g., a CD40 ligand-Ig (CD40L-Ig) fusion, or a fragment thereof that binds CD40); or an agonist of CD27 (e.g., a CD27 ligand-Ig (e.g. CD70L-Ig) fusion, or a fragment thereof that binds CD40).

ICOS is an inducible T cell costimulatory receptor molecule that displays some homology to CD28 and CTLA-4, and interacts with B7-H2 expressed on the surface of antigen-presenting cells. ICOS has been implicated in the regulation of cell-mediated and humoral immune responses.

4-1BB is a type 2 transmembrane glycoprotein belonging to the TNF superfamily, and is expressed on activated T Lymphocytes.

OX40 (also referred to as CD134 or TNFRSF4) is a T cell costimulatory molecule that is engaged by OX40L, and frequently is induced in antigen presenting cells and other cell types. OX40 is known to enhance cytokine expression and survival of effector T cells.

GITR (TNFRSF18) is a T cell costimulatory molecule that is engaged by GITRL and is preferentially expressed in FoxP3+ regulatory T cells. GITR plays a significant role in the maintenance and function of Treg within the tumor microenvironment.

TNFRSF25 is a T cell costimulatory molecule that is preferentially expressed in CD4+ and CD8+ T cells following antigen stimulation. Signaling through TNFRSF25 is provided by TL1A, and functions to enhance T cell sensitivity to IL-2 receptor mediated proliferation in a cognate antigen dependent manner.

CD40 is a costimulatory protein found on various antigen presenting cells which plays a role in their activation. The binding of CD40L (CD154) on TH cells to CD40 activates antigen presenting cells and induces a variety of downstream effects.

CD27 a T cell costimulatory molecule belonging to the TNF superfamily which plays a role in the generation and long-term maintenance of T cell immunity. It binds to a ligand CD70 in various immunological processes.

Additional costimulatory molecules that may be utilized in the present invention include, but are not limited to, HVEM, CD28, CD30, CD30L, CD40, CD70, LIGHT (CD258), B7-1, and B7-2.

As for the gp96-Ig fusions, the Ig portion (“tag”) of the T cell costimulatory fusion protein can contain, a non-variable portion of an immunoglobulin molecule (e.g., an IgG1, IgG2, IgG3, IgG4, IgM, IgA, or IgE molecule). As described above, such portions typically contain at least functional CH2 and CH3 domains of the constant region of an immunoglobulin heavy chain. In some embodiments, a T cell costimulatory peptide can be fused to the hinge, CH2 and CH3 domains of murine IgG1 (Bowen et al., J Immunol 1996, 156:442-449). The Ig tag can be from a mammalian (e.g., human, mouse, monkey, or rat) immunoglobulin, but human immunoglobulin can be particularly useful when the fusion protein is intended for in vivo use for humans. Again, DNAs encoding immunoglobulin light or heavy chain constant regions are known or readily available from cDNA libraries. Various leader sequences as described above also can be used for secretion of T cell costimulatory fusion proteins from bacterial and mammalian cells.

A representative nucleotide sequence (SEQ ID NO:4) encoding the extracellular domain of human ICOSL fused to Ig, and the amino acid sequence of the encoded fusion (SEQ ID NO:5) are provided:

(SEQ ID NO: 4)
ATGAGACTGGGAAGCCCTGGCCTGCTGTTTCTGCTGTTCAGCAGCCTGA
GAGCCGACACCCAGGAAAAAGAAGTGCGGGCCATGGTGGGAAGCGACGT
GGAACTGAGCTGCGCCTGTCCTGAGGGCAGCAGATTCGACCTGAACGAC
GTGTACGTGTACTGGCAGACCAGCGAGAGCAAGACCGTCGTGACCTACC
ACATCCCCCAGAACAGCTCCCTGGAAAACGTGGACAGCCGGTACAGAAA
CCGGGCCCTGATGTCTCCTGCCGGCATGCTGAGAGGCGACTTCAGCCTG
CGGCTGTTCAACGTGACCCCCCAGGACGAGCAGAAATTCCACTGCCTGG
TGCTGAGCCAGAGCCTGGGCTTCCAGGAAGTGCTGAGCGTGGAAGTGAC
CCTGCACGTGGCCGCCAATTTCAGCGTGCCAGTGGTGTCTGCCCCCCAC
AGCCCTTCTCAGGATGAGCTGACCTTCACCTGTACCAGCATCAACGGCT
ACCCCAGACCCAATGTGTACTGGATCAACAAGACCGACAACAGCCTGCT
GGACCAGGCCCTGCAGAACGATACCGTGTTCCTGAACATGCGGGGCCTG
TACGACGTGGTGTCCGTGCTGAGAATCGCCAGAACCCCCAGCGTGAACA
TCGGCTGCTGCATCGAGAACGTGCTGCTGCAGCAGAACCTGACCGTGGG
CAGCCAGACCGGCAACGACATCGGCGAGAGAGACAAGATCACCGAGAAC
CCCGTGTCCACCGGCGAGAAGAATGCCGCCACCTCTAAGTACGGCCCTC
CCTGCCCTTCTTGCCCAGCCCCTGAATTTCTGGGCGGACCCTCCGTGTT
TCTGTTCCCCCCAAAGCCCAAGGACACCCTGATGATCAGCCGGACCCCC
GAAGTGACCTGCGTGGTGGTGTGTCCCAGGAAGATCCCGAGGTGCAGTT
CAATTGGTACGTGGACGGGGTGGAAGTGCACAACGCCAAGACCAAGCCC
AGAGAGGAACAGTTCAACAGCACCTACCGGGTGGTGTCTGTGCTGACCG
TGCTGCACCAGGATTGGCTGAGCGGCAAAGAGTACAAGTGCAAGGTGTC
CAGCAAGGGCCTGCCCAGCAGCATCGAAAAGACCATCAGCAACGCCACC
GGCCAGCCCAGGGAACCCCAGGTGTACACACTGCCCCCTAGCCAGGAAG
AGATGACCAAGAACCAGGTGTCCCTGACCTGTCTCGTGAAGGGCTTCTA
CCCCTCCGATATCGCCGTGGAATGGGAGAGCAACGGCCAGCCAGAGAAC
AACTACAAGACCACCCCCCCAGTGCTGGACAGCGACGGCTCATTCTTCC
TGTACTCCCGGCTGACAGTGGACAAGAGCAGCTGGCAGGAAGGCAACGT
GTTCAGCTGCAGCGTGATGCACGAAGCCCTGCACAACCACTACACCCAG
AAGTCCCTGTCTCTGTCCCTGGGCAAATGA.
(SEQ ID NO: 5)
MRLGSPGLLFLLFSSLRADTQEKEVRAMVGSDVELSCACPEGSRFDLND
VYVYWQTSESKTVVTYHIPQNSSLENVDSRYRNRALMSPAGMLRGDFSL
RLFNVTPQDEQKFHCLVLSQSLGFQEVLSVEVTLHVAANFSVPVVSAPH
SPSQDELTFICTSINGYPRPNVYWINKTDNSLLDQALQNDTVFLNMRGL
YDVVSVLRIARTPSVNIGCCIENVLLQQNLTVGSQTGNDIGERDKITEN
PVSTGEKNAATSKYGPPCPSCPAPEFLGGPSVFLFPPKPKDTLMISRTP
EVTCVVVDVSQEDPEVQFNWYVDGVEVHNAKTKPREEQFNSTYRVVSVL
TVLHQDWLSGKEYKCKVSSKGLPSSIEKTISNATGQPREPQVYTLPPSQ
EEMTKNQVSLTCLVKGFYPSDIAVEWESNGQPENNYKTTPPVLDSDGSF
FLYSRLTVDKSSWQEGNVFSCSVMHEALHNHYTQKSLSLSLGK.

A representative nucleotide sequence (SEQ ID NO:6) encoding the extracellular domain of human 4-1BBL fused to Ig, and the encoded amino acid sequence (SEQ ID NO:7) are provided:

(SEQ ID NO: 6)
ATGTCTAAGTACGGCCCTCCCTGCCCTAGCTGCCCTGCCCCTGAATTTC
TGGGCGGACCCAGCGTGTTCCTGTTCCCCCCAAAGCCCAAGGACACCCT
GATGATCAGCCGGACCCCCGAAGTGACCTGCGTGGTGGTGGATGTGTCC
CAGGAAGATCCCGAGGTGCAGTTCAATTGGTACGTGGACGGCGTGGAAG
TGCACAACGCCAAGACCAAGCCCAGAGAGGAACAGTTCAACAGCACCTA
CCGGGTGGTGTCCGTGCTGACCGTGCTGCACCAGGATTGGCTGAGCGGC
AAAGAGTACAAGTGCAAGGTGTCCAGCAAGGGCCTGCCCAGCAGCATCG
AGAAAACCATCAGCAACGCCACCGGCCAGCCCAGGGAACCCCAGGTGTA
CACACTGCCCCCTAGCCAGGAAGAGATGACCAAGAACCAGGTGTCCCTG
ACCTGTCTCGTGAAGGGCTTCTACCCCTCCGATATCGCCGTGGAATGGG
AGAGCAACGGCCAGCCTGAGAACAACTACAAGACCACCCCCCCAGTGCT
GGACAGCGACGGCTCATTCTTCCTGTACAGCAGACTGACCGTGGACAAG
AGCAGCTGGCAGGAAGGCAACGTGTTCAGCTGCAGCGTGATGCACGAGG
CCCTGCACAACCACTACACCCAGAAGTCCCTGTCTCTGAGCCTGGGCAA
GGCCTGTCCATGGGCTGTGTCTGGCGCTAGAGCCTCTCCTGGATCTGCC
GCCAGCCCCAGACTGAGAGAGGGACCTGAGCTGAGCCCCGATGATCCTG
CCGGACTGCTGGATCTGAGACAGGGCATGTTCGCCCAGCTGGTGGCCCA
GAACGTGCTGCTGATCGATGGCCCCCTGAGCTGGTACAGCGATCCTGGA
CTGGCTGGCGTGTCACTGACAGGCGGCCTGAGCTACAAAGAGGACACCA
AAGAACTGGTGGTGGCCAAGGCCGGCGTGTACTACGTGTTCTTTCAGCT
GGAACTGCGGAGAGTGGTGGCCGGCGAAGGATCCGGCTCTGTGTCTCTG
GCTCTGCATCTGCAGCCCCTGAGATCTGCTGCTGGCGCTGCTGCTCTGG
CCCTGACAGTGGACCTGCCTCCTGCCTCTAGCGAGGCCAGAAACAGCGC
ATTCGGGTTTCAAGGCAGACTGCTGCACCTGTCTGCCGGCCAGAGACTG
GGAGTGCATCTGCACACAGAGGCCAGAGCCAGGCACGCCTGGCAGCTGA
CTCAGGGCGCTACAGTGCTGGGCCTGTTCAGAGTGACCCCCGAGATTCC
AGCCGGCCTGCCTAGCCCCAGATCCGAATGA
(SEQ ID NO: 7)
MSKYGPPCPSCPAPEFLGGPSVFLFPPKPKDTLMISRTPETCVVVDVSQ
EDPEVQFNWYVDGVEVHNAKTKPREEQFNSTYRVVSVLTVLHQDWLSGK
EYKCKVSSKGLPSSIEKTISNATGQPREPQVYTLPPSQEEMTKNQVSLT
CLVKGFYPSDIAVEWESNGQPENNYKTTPPVLDSDGSFFLYSRLTVDKS
SWQEGNVFSCSVMHEALHNHYTQKSLSLSLGKACPWAVSGARASPGSAA
SPRLREGPELSPDDPAGLLDLRQGMFAQLVAQNVLLIDGPLSWYSDPGL
AGVSLTGGLSYKEDTKELWAKAGVYYVFFQLELRRVVAGEGSGSVSLAL
HLQPLRSAAGAAALALTVDLPPASSEARNSAFGFQGRLLHLSAGQRLGV
HLHTEARARHAWQLTQGATVLGLFRVTPEIPAGLPSPRSE.

A representative nucleotide sequence (SEQ ID NO:8) encoding the extracellular domain of human TL1A fused to Ig, and the encoded amino acid sequence (SEQ ID NO:9) are provided:

(SEQ ID NO: 8)
ATGTCTAAGTACGGCCCTCCCTGCCCTAGCTGCCCTGCCCCTGAATTTC
TGGGCGGACCCAGCGTGTTCCTGTTCCCCCCAAAGCCCAAGGACACCCT
GATGATCAGCCGGACCCCCGAAGTGACCTGCGTGGTGGTGGATGTGTCC
CAGGAAGATCCCGAGGTGCAGTTCAATTGGTACGTGGACGGCGTGGAAG
TGCACAACGCCAAGACCAAGCCCAGAGAGGAACAGTTCAACAGCACCTA
CCGGGTGGTGTCCGTGCTGACCGTGCTGCACCAGGATTGGCTGAGCGGC
AAAGAGTACAAGTGCAAGGTGTCCAGCAAGGGCCTGCCCAGCAGCATCG
AGAAAACCATCAGCAACGCCACCGGCCAGCCCAGGGAACCCCAGGTGTA
CACACTGCCCCCTAGCCAGGAAGAGATGACCAAGAACCAGGTGTCCCTG
ACCTGTCTCGTGAAGGGCTTCTACCCCTCCGATATCGCCGTGGAATGGG
AGAGCAACGGCCAGCCTGAGAACAACTACAAGACCACCCCCCCAGTGCT
GGACAGCGACGGCTCATTCTTCCTGTACAGCAGACTGACCGTGGACAAG
AGCAGCTGGCAGGAAGGCAACGTGTTCAGCTGCAGCGTGATGCACGAGG
CCCTGCACAACCACTACACCCAGAAGTCCCTGTCTCTGAGCCTGGGCAA
GATCGAGGGCCGGATGGATAGAGCCCAGGGCGAAGCCTGCGTGCAGTTC
CAGGCTCTGAAGGGCCAGGAATTCGCCCCCAGCCACCAGCAGGTGTACG
CCCCTCTGAGAGCCGACGGCGATAAGCCTAGAGCCCACCTGACAGTCGT
GCGGCAGACCCCTACCCAGCACTTCAAGAATCAGTTCCCCGCCCTGCAC
TGGGAGCACGAACTGGGCCTGGCCTTCACCAAGAACAGAATGAACTACA
CCAACAAGTTTCTGCTGATCCCCGAGAGCGGCGACTACTTCATCTACAG
CCAAGTGACCTTCCGGGGCATGACCAGCGAGTGCAGCGAGATCAGACAG
GCCGGCAGACCTAACAAGCCCGACAGCATCACCGTCGTGATCACCAAAG
TGACCGACAGCTACCCCGAGCCCACCCAGCTGCTGATGGGCACCAAGAG
CGTGTGCGAAGTGGGCAGCAACTGGTTCCAGCCCATCTACCTGGGCGCC
ATGTTTAGTCTGCAAGAGGGCGACAAGCTGATGGTCAACGTGTCCGACA
TCAGCCTGGTGGATTACACCAAAGAGGACAAGACCTTCTTCGGCGCCTT
TCTGCTCTGA
(SEQ ID NO: 9)
MSKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLSG
KEYKCKVSSKGLPSSIEKTISNATGQPREPQVYTLPPSQEEMTKNQVSL
TCLVKGFYPSDIAVEWESNGQPENNYKTTPPVLDSDGSFFLYSRLTVDK
SSWQEGNVFSCSVMHEALHNHYTQKSLSLSLGKIEGRMDRAQGEACVQF
QALKGQEFAPSHQQVYAPLRADGDKPRAHLTWRQTPTQHFKNQFPALHW
EHELGLAFTKNRMNYTNKFLLIPESGDYFIYSQVTFRGMTSECSEIRQA
GRPNKPDSITWITKVTDSYPEPTQLLMGTKSVCEVGSNWFQPIYLGAMF
SLQEGDKLMVNVSDISLVDYTKEDKTFFGAFLL.

A representative nucleotide sequence (SEQ ID NO:10) encoding human OX40L-Ig, and the encoded amino acid sequence (SEQ ID NO:11) are provided:

(SEQ ID NO:10)
ATGTCTAAGTACGGCCCTCCCTGCCCTAGCTGCCCTGCCCCTGAATTTC
TGGGCGGACCCAGCGTGTTCCTGTTCCCCCCAAAGCCCAAGGACACCCT
GATGATCAGCCGGACCCCCGAAGTGACCTGCGTGGTGGTGGATGTGTCC
CAGGAAGATCCCGAGGTGCAGTTCAATTGGTACGTGGACGGCGTGGAAG
TGCACAACGCCAAGACCAAGCCCAGAGAGGAACAGTTCAACAGCACCTA
CCGGGTGGTGTCCGTGCTGACCGTGCTGCACCAGGATTGGCTGAGCGGC
AAAGAGTACAAGTGCAAGGTGTCCAGCAAGGGCCTGCCCAGCAGCATCG
AGAAAACCATCAGCAACGCCACCGGCCAGCCCAGGGAACCCCAGGTGTA
CACACTGCCCCCTAGCCAGGAAGAGATGACCAAGAACCAGGTGTCCCTG
ACCTGTCTCGTGAAGGGCTTCTACCCCTCCGATATCGCCGTGGAATGGG
AGAGCAACGGCCAGCCTGAGAACAACTACAAGACCACCCCCCCAGTGCT
GGACAGCGACGGCTCATTCTTCCTGTACAGCAGACTGACCGTGGACAAG
AGCAGCTGGCAGGAAGGCAACGTGTTCAGCTGCAGCGTGATGCACGAGG
CCCTGCACAACCACTACACCCAGAAGTCCCTGTCTCTGAGCCTGGGCAA
GATCGAGGGCCGGATGGATCAGGTGTCACACAGATACCCCCGGATCCAG
AGCATCAAAGTGCAGTTTACCGAGTACAAGAAAGAGAAGGGCTTTATCC
TGACCAGCCAGAAAGAGGACGAGATCATGAAGGTGCAGAACAACAGCGT
GATCATCAACTGCGACGGGTTCTACCTGATCAGCCTGAAGGGCTACTTC
AGTCAGGAAGTGAACATCAGCCTGCACTACCAGAAGGACGAGGAACCCC
TGTTCCAGCTGAAGAAAGTGCGGAGCGTGAACAGCCTGATGGTGGCCTC
TCTGACCTACAAGGACAAGGTGTACCTGAACGTGACCACCGACAACACC
AGCCTGGACGACTTCCACGTGAACGGCGGCGAGCTGATCCTGATTCACC
AGAACCCCGGCGAGTTCTGCGTGCTCTGA
(SEQ ID NO: 11)
MSKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLSG
KEYKCKVSSKGLPSSIEKTISNATGQPREPQVYTLPPSQEEMTKNQVSL
TCLVKGFYPSDIAVEWESNGQPENNYKTTPPVLDSDGSFFLYSRLTVDK
SSWQEGNVFSCSVMHEALHNHYTQKSLSLSLGKIEGRMDQVSHRYPRIQ
SIKVQFTEYKKEKGFILTSQKEDEIMKVQNNSVIINCDGFYLISLKGYF
SQEVNISLHYQKDEEPLFQLKKVRSVNSLMVASLTYKDKVYLNVTTDNT
SLDDFHVNGGELILIHQNPGEFCVL.

Representative nucleotide and amino acid sequences for human TL1A are set forth in SEQ ID NO:12 and SEQ ID NO:13, respectively:

(SEQ ID NO: 12)
TCCCAAGTAGCTGGGACTACAGGAGCCCACCACCACCCCCGGCTAATTT
TTTGTATTTTTAGTAGAGACGGGGTTTCACCGTGTTAGCCAAGATGGTC
TTGATCACCTGACCTCGTGATCCACCCGCCTTGGCCTCCCAAAGTGCTG
GGATTACAGGCATGAGCCACCGCGCCCGGCCTCCATTCAAGTCTTTATT
GAATATCTGCTATGTTCTACACACTGTTCTAGGTGCTGGGGATGCAACA
GGGGACAAAATAGGCAAAATCCCTGTCCTTTTGGGGTTGACATTCTAGT
GACTCTTCATGTAGTCTAGAAGAAGCTCAGTGAATAGTGTCTGTGGTTG
TTACCAGGGACACAATGACAGGAACATTCTTGGGTAGAGTGAGAGGCCT
GGGGAGGGAAGGGTCTCTAGGATGGAGCAGATGCTGGGCAGTCTTAGGG
AGCCCCTCCTGGCATGCACCCCCTCATCCCTCAGGCCACCCCCGTCCCT
TGCAGGAGCACCCTGGGGAGCTGTCCAGAGCGCTGTGCCGCTGTCTGTG
GCTGGAGGCAGAGTAGGTGGTGTGCTGGGAATGCGAGTGGGAGAACTGG
GATGGACCGAGGGGAGGCGGGTGAGGAGGGGGGCAACCACCCAACACCC
ACCAGCTGCTTTCAGTGTTCTGGGTCCAGGTGCTCCTGGCTGGCCTTGT
GGTCCCCCTCCTGCTTGGGGCCACCCTGACCTACACATACCGCCACTGC
TGGCCTCACAAGCCCCTGGTTACTGCAGATGAAGCTGGGATGGAGGCTC
TGACCCCACCACCGGCCACCCATCTGTCACCCTTGGACAGCGCCCACAC
CCTTCTAGCACCTCCTGACAGCAGTGAGAAGATCTGCACCGTCCAGTTG
GTGGGTAACAGCTGGACCCCTGGCTACCCCGAGACCCAGGAGGCGCTCT
GCCCGCAGGTGACATGGTCCTGGGACCAGTTGCCCAGCAGAGCTCTTGG
CCCCGCTGCTGCGCCCACACTCTCGCCAGAGTCCCCAGCCGGCTCGCCA
GCCATGATGCTGCAGCCGGGCCCGCAGCTCTACGACGTGATGGACGCGG
TCCCAGCGCGGCGCTGGAAGGAGTTCGTGCGCACGCTGGGGCTGCGCGA
GGCAGAGATCGAAGCCGTGGAGGTGGAGATCGGCCGCTTCCGAGACCAG
CAGTACGAGATGCTCAAGCGCTGGCGCCAGCAGCAGCCCGCGGGCCTCG
GAGCCGTTTACGCGGCCCTGGAGCGCATGGGGCTGGACGGCTGCGTGGA
AGACTTGCGCAGCCGCCTGCAGCGCGGCCCGTGACACGGCGCCCACTTG
CCACCTAGGCGCTCTGGTGGCCCTTGCAGAAGCCCTAAGTACGGTTACT
TATGCGTGTAGACATTTTATGTCACTTATTAAGCCGCTGGCACGGCCCT
GCGTAGCAGCACCAGCCGGCCCCACCCCTGCTCGCCCCTATCGCTCCAG
CCAAGGCGAAGAAGCACGAACGAATGTCGAGAGGGGGTGAAGACATTTC
TCAACTTCTCGGCCGGAGTTTGGCTGAGATCGCGGTATTAAATCTGTGA
AAGAAAACAAAACAAAACAA
(SEQ ID NO: 13)
MEQRPRGCAAVAAALLLVLLGARAQGGTRSPRCDCAGDFHKKIGLFCCR
GCPAGHYLKAPCTEPCGNSTCLVCPQDTFLAWENHHNSECARCQACDEQ
ASQVALENCSAVADTRCGCKPGWFVECQVSQCVSSSPFYCQPCLDCGAL
HRHTRLLCSRRDTDCGTCLPGFYEHGDGCVSCPTPPPSLAGAPWGAVQS
AVPLSVAGGRVGVFWVQVLLAGLVVPLLLGATLTYTYRHCWPHKPLVTA
DEAGMEALTPPPATHLSPLDSAHTLLAPPDSSEKICTVQLVGNSWTPGY
PETQEALCPQVTWSWDQLPSRALGPAAAPTLSPESPAGSPAMMLQPGPQ
LYDVMDAVPARRWKEFVRTLGLREAEIEAVEVEIGRFRDQQYEMLKRWR
QQQPAGLGAVYAALERMGLDGCVEDLRSRLQRGP.

Representative nucleotide and amino acid sequences for human HVEM are set forth in SEQ ID NO:26 (accession no CR456909) and SEQ ID NO:27, respectively (accession no CR456909):

(SEQ ID NO: 26)
ATGGAGCCTCCTGGAGACTGGGGGCCTCCTCCCTGGAGATCCACCCCCA
AAACCGACGTCTTGAGGCTGGTGCTGTATCTCACCTTCCTGGGAGCCCC
CTGCTACGCCCCAGCTCTGCCGTCCTGCAAGGAGGACGAGTACCCAGTG
GGCTCCGAGTGCTGCCCCAAGTGCAGTCCAGGTTATCGTGTGAAGGAGG
CCTGCGGGGAGCTGACGGGCACAGTGTGTGAACCCTGCCCTCCAGGCAC
CTACATTGCCCACCTCAATGGCCTAAGCAAGTGTCTGCAGTGCCAAATG
TGTGACCCAGCCATGGGCCTGCGCGCGAGCCGGAACTGCTCCAGGACAG
AGAACGCCGTGTGTGGCTGCAGCCCAGGCCACTTCTGCATCGTCCAGGA
CGGGGACCACTGCGCCGCGTGCCGCGCTTACGCCACCTCCAGCCCGGGC
CAGAGGGTGCAGAAGGGAGGCACCGAGAGTCAGGACACCCTGTGTCAGA
ACTGCCCCCCGGGGACCTTCTCTCCCAATGGGACCCTGGAGGAATGTCA
GCACCAGACCAAGTGCAGCTGGCTGGTGACGAAGGCCGGAGCTGGGACC
AGCAGCTCCCACTGGGTATGGTGGTTTCTCTCAGGGAGCCTCGTCATCG
TCATTGTTTGCTCCACAGTTGGCCTAATCATATGTGTGAAAAGAAGAAA
GCCAAGGGGTGATGTAGTCAAGGTGATCGTCTCCGTCCAGCGGAAAAGA
CAGGAGGCAGAAGGTGAGGCCACAGTCATTGAGGCCCTGCAGGCCCCTC
CGGACGTCACCACGGTGGCCGTGGAGGAGACAATACCCTCATTCACGGG
GAGGAGCCCAAACCATTAA
(SEQ ID NO: 27)
MEPPGDWGPPPWRSTPKTDVLRLVLYLTFLGAPCYAPALPSCKEDEYPV
GSECCPKCSPGYRVKEACGELTGTVCEPCPPGTYIAHLNGLSKCLQCQM
CDPAMGLRASRNCSRTENAVCGCSPGHFCIVQDGDHCAACRAYATSSPG
QRVQKGGTESQDTLCQNCPPGTFSPNGTLEECQHQTKCSWLVTKAGAGT
SSSHWVWWFLSGSLVIVIVCSTVGLIICVKRRKPRGDVVKVIVSVQRKR
QEAEGEATVIEALQAPPDVTTVAVEETIPSFTGRSPNH.

Representative nucleotide and amino acid sequences for human CD28 are set forth in SEQ ID NO:28 (accession no. NM_006139) and SEQ ID NO:29, respectively:

(SEQ ID NO: 28)
TAAAGTCATCAAAACAACGTTATATCCTGTGTGAAATGCTGCAGTCAGG
ATGCCTTGTGGTTTGAGTGCCTTGATCATGTGCCCTAAGGGGATGGTGG
CGGTGGTGGTGGCCGTGGATGACGGAGACTCTCAGGCCTTGGCAGGTGC
GTCTTTCAGTTCCCCTCACACTTCGGGTTCCTCGGGGAGGAGGGGCTGG
AACCCTAGCCCATCGTCAGGACAAAGATGCTCAGGCTGCTCTTGGCTCT
CAACTTATTCCCTTCAATTCAAGTAACAGGAAACAAGATTTTGGTGAAG
CAGTCGCCCATGCTTGTAGCGTACGACAATGCGGTCAACCTTAGCTGCA
AGTATTCCTACAATCTCTTCTCAAGGGAGTTCCGGGCATCCCTTCACAA
AGGACTGGATAGTGCTGTGGAAGTCTGTGTTGTATATGGGAATTACTCC
CAGCAGCTTCAGGTTTACTCAAAAACGGGGTTCAACTGTGATGGGAAAT
TGGGCAATGAATCAGTGACATTCTACCTCCAGAATTTGTATGTTAACCA
AACAGATATTTACTTCTGCAAAATTGAAGTTATGTATCCTCCTCCTTAC
CTAGACAATGAGAAGAGCAATGGAACCATTATCCATGTGAAAGGGAAAC
ACCTTTGTCCAAGTCCCCTATTTCCCGGACCTTCTAAGCCCTTTTGGGT
GCTGGTGGTGGTTGGTGGAGTCCTGGCTTGCTATAGCTTGCTAGTAACA
GTGGCCTTTATTATTTTCTGGGTGAGGAGTAAGAGGAGCAGGCTCCTGC
ACAGTGACTACATGAACATGACTCCCCGCCGCCCCGGGCCCACCCGCAA
GCATTACCAGCCCTATGCCCCACCACGCGACTTCGCAGCCTATCGCTCC
TGACACGGACGCCTATCCAGAAGCCAGCCGGCTGGCAGCCCCCATCTGC
TCAATATCACTGCTCTGGATAGGAAATGACCGCCATCTCCAGCCGGCCA
CCTCAGGCCCCTGTTGGGCCACCAATGCCAATTTTTCTCGAGTGACTAG
ACCAAATATCAAGATCATTTTGAGACTCTGAAATGAAGTAAAAGAGATT
TCCTGTGACAGGCCAAGTCTTACAGTGCCATGGCCCACATTCCAACTTA
CCATGTACTTAGTGACTTGACTGAGAAGTTAGGGTAGAAAACAAAAAGG
GAGTGGATTCTGGGAGCCTCTTCCCTTTCTCACTCACCTGCACATCTCA
GTCAAGCAAAGTGTGGTATCCACAGACATTTTAGTTGCAGAAGAAAGGC
TAGGAAATCATTCCTTTTGGTTAAATGGGTGTTTAATCTTTTGGTTAGT
GGGTTAAACGGGGTAAGTTAGAGTAGGGGGAGGGATAGGAAGACATATT
TAAAAACCATTAAAACACTGTCTCCCACTCATGAAATGAGCCACGTAGT
TCCTATTTAATGCTGTTTTCCTTTAGTTTAGAAATACATAGACATTGTC
TTTTATGAATTCTGATCATATTTAGTCATTTTGACCAAATGAGGGATTT
GGTCAAATGAGGGATTCCCTCAAAGCAATATCAGGTAAACCAAGTTGCT
TTCCTCACTCCCTGTCATGAGACTTCAGTGTTAATGTTCACAATATACT
TTCGAAAGAATAAAATAGTTCTCCTACATGAAGAAAGAATATGTCAGGA
AATAAGGTCACTTTATGTCAAAATTATTTGAGTACTATGGGACCTGGCG
CAGTGGCTCATGCTTGTAATCCCAGCACTTTGGGAGGCCGAGGTGGGCA
GATCACTTGAGATCAGGACCAGCCTGGTCAAGATGGTGAAACTCCGTCT
GTACTAAAAATACAAAATTTAGCTTGGCCTGGTGGCAGGCACCTGTAAT
CCCAGCTGCCCAAGAGGCTGAGGCATGAGAATCGCTTGAACCTGGCAGG
CGGAGGTTGCAGTGAGCCGAGATAGTGCCACAGCTCTCCAGCCTGGGCG
ACAGAGTGAGACTCCATCTCAAACAACAACAACAACAACAACAACAACA
ACAAACCACAAAATTATTTGAGTACTGTGAAGGATTATTTGTCTAACAG
TTCATTCCAATCAGACCAGGTAGGAGCTTTCCTGTTTCATATGTTTCAG
GGTTGCACAGTTGGTCTCTTTAATGTCGGTGTGGAGATCCAAAGTGGGT
TGTGGAAAGAGCGTCCATAGGAGAAGTGAGAATACTGTGAAAAAGGGAT
GTTAGCATTCATTAGAGTATGAGGATGAGTCCCAAGAAGGTTCTTTGGA
AGGAGGACGAATAGAATGGAGTAATGAAATTCTTGCCATGTGCTGAGGA
GATAGCCAGCATTAGGTGACAATCTTCCAGAAGTGGTCAGGCAGAAGGT
GCCCTGGTGAGAGCTCCTTTACAGGGACTTTATGTGGTTTAGGGCTCAG
AGCTCCAAAACTCTGGGCTCAGCTGCTCCTGTACCTTGGAGGTCCATTC
ACATGGGAAAGTATTTTGGAATGTGTCTTTTGAAGAGAGCATCAGAGTT
CTTAAGGGACTGGGTAAGGCCTGACCCTGAAATGACCATGGATATTTTT
CTACCTACAGTTTGAGTCAACTAGAATATGCCTGGGGACCTTGAAGAAT
GGCCCTTCAGTGGCCCTCACCATTTGTTCATGCTTCAGTTAATTCAGGT
GTTGAAGGAGCTTAGGTTTTAGAGGCACGTAGACTTGGTTCAAGTCTCG
TTAGTAGTTGAATAGCCTCAGGCAAGTCACTGCCCACCTAAGATGATGG
TTCTTCAACTATAAAATGGAGATAATGGTTACAAATGTCTCTTCCTATA
GTATAATCTCCATAAGGGCATGGCCCAAGTCTGTCTTTGACTCTGCCTA
TCCCTGACATTTAGTAGCATGCCCGACATACAATGTTAGCTATTGGTAT
TATTGCCATATAGATAAATTATGTATAAAAATTAAACTGGGCAATAGCC
TAAGAAGGGGGGAATATTGTAACACAAATTTAAACCCACTACGCAGGGA
TGAGGTGCTATAATATGAGGACCTTTTAACTTCCATCATTTTCCTGTTT
CTTGAAATAGTTTATCTTGTAATGAAATATAAGGCACCTCCCACTTTTA
TGTATAGAAAGAGGTCTTTTAATTTTTTTTTAATGTGAGAAGGAAGGGA
GGAGTAGGAATCTTGAGATTCCAGATCGAAAATACTGTACTTTGGTTGA
TTTTTAAGTGGGCTTCCATTCCATGGATTTAATCAGTCCCAAGAAGATC
AAACTCAGCAGTACTTGGGTGCTGAAGAACTGTTGGATTTACCCTGGCA
CGTGTGCCACTTGCCAGCTTCTTGGGCACACAGAGTTCTTCAATCCAAG
TTATCAGATTGTATTTGAAAATGACAGAGCTGGAGAGTTTTTTGAAATG
GCAGTGGCAAATAAATAAATACTTTTTTTTAAATGGAAAGACTTGATCT
ATGGTAATAAATGATTTTGTTTTCTGACTGGAAAAATAGGCCTACTAAA
GATGAATCACACTTGAGATGTTTCTTACTCACTCTGCACAGAAACAAAG
AAGAAATGTTATACAGGGAAGTCCGTTTTCACTATTAGTATGAACCAAG
AAATGGTTCAAAAACAGTGGTAGGAGCAATGCTTTCATAGTTTCAGATA
TGGTAGTTATGAAGAAAACAATGTCATTTGCTGCTATTATTGTAAGAGT
CTTATAATTAATGGTACTCCTATAATTTTTGATTGTGAGCTCACCTATT
TGGGTTAAGCATGCCAATTTAAAGAGACCAAGTGTATGTACATTATGTT
CTACATATTCAGTGATAAAATTACTAAACTACTATATGTCTGCTTTAAA
TTTGTACTTTAATATTGTCTTTTGGTATTAAGAAAGATATGCTTTCAGA
ATAGATATGCTTCGCTTTGGCAAGGAATTTGGATAGAACTTGCTATTTA
AAAGAGGTGTGGGGTAAATCCTTGTATAAATCTCCAGTTTAGCCTTTTT
TGAAAAAGCTAGACTTTCAAATACTAATTTCACTTCAAGCAGGGTACGT
TTCTGGTTTGTTTGCTTGACTTCAGTCACAATTTCTTATCAGACCAATG
GCTGACCTCTTTGAGATGTCAGGCTAGGCTTACCTATGTGTTCTGTGTC
ATGTGAATGCTGAGAAGTTTGACAGAGATCCAACTTCAGCCTTGACCCC
ATCAGTCCCTCGGGTTAACTAACTGAGCCACCGGTCCTCATGGCTATTT
TAATGAGGGTATTGATGGTTAAATGCATGTCTGATCCCTTATCCCAGCC
ATTTGCACTGCCAGCTGGGAACTATACCAGACCTGGATACTGATCCCAA
AGTGTTAAATTCAACTACATGCTGGAGATTAGAGATGGTGCCAATAAAG
GACCCAGAACCAGGATCTTGATTGCTATAGACTTATTAATAATCCAGGT
CAAAGAGAGTGACACACACTCTCTCAAGACCTGGGGTGAGGGAGTCTGT
GTTATCTGCAAGGCCATTTGAGGCTCAGAAAGTCTCTCTTTCCTATAGA
TATATGCATACTTTCTGACATATAGGAATGTATCAGGAATACTCAACCA
TCACAGGCATGTTCCTACCTCAGGGCCTTTACATGTCCTGTTTACTCTG
TCTAGAATGTCCTTCTGTAGATGACCTGGCTTGCCTCGTCACCCTTCAG
GTCCTTGCTCAAGTGTCATCTTCTCCCCTAGTTAAACTACCCCACACCC
TGTCTGCTTTCCTTGCTTATTTTTCTCCATAGCATTTTACCATCTCTTA
CATTAGACATTTTTCTTATTTATTTGTAGTTTATAAGCTTCATGAGGCA
AGTAACTTTGCTTTGTTTCTTGCTGTATCTCCAGTGCCCAGAGCAGTGC
CTGGTATATAATAAATATTTATTGACTGAGTGAAAAAAAAAAAAAAAAA
(SEQ ID NO: 29)
MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS.

Representative nucleotide and amino acid sequences for human CD30L are set forth in SEQ ID NO:30 (accession no. L09753) and SEQ ID NO:31, respectively:

(SEQ ID NO: 30)
CCAAGTCACATGATTCAGGATTCAGGGGGAGAATCCTTCTTGGAACAGA
GATGGGCCCAGAACTGAATCAGATGAAGAGAGATAAGGTGTGATGTGGG
GAAGACTATATAAAGAATGGACCCAGGGCTGCAGCAAGCACTCAACGGA
ATGGCCCCTCCTGGAGACACAGCCATGCATGTGCCGGCGGGCTCCGTGG
CCAGCCACCTGGGGACCACGAGCCGCAGCTATTTCTATTTGACCACAGC
CACTCTGGCTCTGTGCCTTGTCTTCACGGTGGCCACTATTATGGTGTTG
GTCGTTCAGAGGACGGACTCCATTCCCAACTCACCTGACAACGTCCCCC
TCAAAGGAGGAAATTGCTCAGAAGACCTCTTATGTATCCTGAAAAGAGC
TCCATTCAAGAAGTCATGGGCCTACCTCCAAGTGGCAAAGCATCTAAAC
AAAACCAAGTTGTCTTGGAACAAAGATGGCATTCTCCATGGAGTCAGAT
ATCAGGATGGGAATCTGGTGATCCAATTCCCTGGTTTGTACTTCATCAT
TTGCCAACTGCAGTTTCTTGTACAATGCCCAAATAATTCTGTCGATCTG
AAGTTGGAGCTTCTCATCAACAAGCATATCAAAAAACAGGCCCTGGTGA
CAGTGTGTGAGTCTGGAATGCAAACGAAACACGTATACCAGAATCTCTC
TCAATTCTTGCTGGATTACCTGCAGGTCAACACCACCATATCAGTCAAT
GTGGATACATTCCAGTACATAGATACAAGCACCTTTCCTCTTGAGAATG
TGTTGTCCATCTTCTTATACAGTAATTCAGACTGAACAGTTTCTCTTGG
CCTTCAGGAAGAAAGCGCCTCTCTACCATACAGTATTTCATCCCTCCAA
ACACTTGGGCAAAAAGAAAACTTTAGACCAAGACAAACTACACAGGGTA
TTAAATAGTATACTTCTCCTTCTGTCTCTTGGAAAGATACAGCTCCAGG
GTTAAAAAGAGAGTTTTTAGTGAAGTATCTTTCAGATAGCAGGCAGGGA
AGCAATGTAGTGTGGTGGGCAGAGCCCCACACAGAATCAGAAGGGATGA
ATGGATGTCCCAGCCCAACCACTAATTCACTGTATGGTCTTGATCTATT
TCTTCTGTTTTGAGAGCCTCCAGTTAAAATGGGGCTTCAGTACCAGAGC
AGCTAGCAACTCTGCCCTAATGGGAAATGAAGGGGAGCTGGGTGTGAGT
GTTTACACTGTGCCCTTCACGGGATACTTCTTTTATCTGCAGATGGCCT
AATGCTTAGTTGTCCAAGTCGCGATCAAGGACTCTCTCACACAGGAAAC
TTCCCTATACTGGCAGATACACTTGTGACTGAACCATGCCCAGTTTATG
CCTGTCTGACTGTCACTCTGGCACTAGGAGGCTGATCTTGTACTCCATA
TGACCCCACCCCTAGGAACCCCCAGGGAAAACCAGGCTCGGACAGCCCC
CTGTTCCTGAGATGGAAAGCACAAATTTAATACACCACCACAATGGAAA
ACAAGTTCAAAGACTTTTACTTACAGATCCTGGACAGAAAGGGCATAAT
GAGTCTGAAGGGCAGTCCTCCTTCTCCAGGTTACATGAGGCAGGAATAA
GAAGTCAGACAGAGACAGCAAGACAGTTAACAACGTAGGTAAAGAAATA
GGGTGTGGTCACTCTCAATTCACTGGCAAATGCCTGAATGGTCTGTCTG
AAGGAAGCAACAGAGAAGTGGGGAATCCAGTCTGCTAGGCAGGAAAGAT
GCCTCTAAGTTCTTGTCTCTGGCCAGAGGTGTGGTATAGAACCAGAAAC
CCATATCAAGGGTGACTAAGCCCGGCTTCCGGTATGAGAAATTAAACTT
GTATACAAAATGGTTGCCAAGGCAACATAAAATTATAAGAATTC
(SEQ ID NO: 31)
MDPGLQQALNGMAPPGDTAMHVPAGSVASHLGTTSRSYFYLTTATLALC
LVFTVATIMVLWQRTDSIPNSPDNVPLKGGNCSEDLLCILKRAPFKKSW
AYLQVAKHLNKTKLSWNKDGILHGVRYQDGNLVIQFPGLYFIICQLQFL
VQCPNNSVDLKLELLINKHIKKQALVTVCESGMQTKHVYQNLSQFLLDY
LQVNTTISVNVDTFQYIDTSTFPLENVLSIFLYSNSD.

Representative nucleotide and amino acid sequences for human CD40 are set forth in SEQ ID NO:32 (accession no. NM_001250) and SEQ ID NO:33, respectively:

(SEQ ID NO: 32)
TTTCCTGGGCGGGGCCAAGGCTGGGGCAGGGGAGTCAGCAGAGGCCTCG
CTCGGGCGCCCAGTGGTCCTGCCGCCTGGTCTCACCTCGCTATGGTTCG
TCTGCCTCTGCAGTGCGTCCTCTGGGGCTGCTTGCTGACCGCTGTCCAT
CCAGAACCACCCACTGCATGCAGAGAAAAACAGTACCTAATAAACAGTC
AGTGCTGTTCTTTGTGCCAGCCAGGACAGAAACTGGTGAGTGACTGCAC
AGAGTTCACTGAAACGGAATGCCTTCCTTGCGGTGAAAGCGAATTCCTA
GACACCTGGAACAGAGAGACACACTGCCACCAGCACAAATACTGCGACC
CCAACCTAGGGCTTCGGGTCCAGCAGAAGGGCACCTCAGAAACAGACAC
CATCTGCACCTGTGAAGAAGGCTGGCACTGTACGAGTGAGGCCTGTGAG
AGCTGTGTCCTGCACCGCTCATGCTCGCCCGGCTTTGGGGTCAAGCAGA
TTGCTACAGGGGTTTCTGATACCATCTGCGAGCCCTGCCCAGTCGGCTT
CTTCTCCAATGTGTCATCTGCTTTCGAAAAATGTCACCCTTGGACAAGC
TGTGAGACCAAAGACCTGGTTGTGCAACAGGCAGGCACAAACAAGACTG
ATGTTGTCTGTGGTCCCCAGGATCGGCTGAGAGCCCTGGTGGTGATCCC
CATCATCTTCGGGATCCTGTTTGCCATCCTCTTGGTGCTGGTCTTTATC
AAAAAGGTGGCCAAGAAGCCAACCAATAAGGCCCCCCACCCCAAGCAGG
AACCCCAGGAGATCAATTTTCCCGACGATCTTCCTGGCTCCAACACTGC
TGCTCCAGTGCAGGAGACTTTACATGGATGCCAACCGGTCACCCAGGAG
GATGGCAAAGAGAGTCGCATCTCAGTGCAGGAGAGACAGTGAGGCTGCA
CCCACCCAGGAGTGTGGCCACGTGGGCAAACAGGCAGTTGGCCAGAGAG
CCTGGTGCTGCTGCTGCTGTGGCGTGAGGGTGAGGGGCTGGCACTGACT
GGGCATAGCTCCCCGCTTCTGCCTGCACCCCTGCAGTTTGAGACAGGAG
ACCTGGCACTGGATGCAGAAACAGTTCACCTTGAAGAACCTCTCACTTC
ACCCTGGAGCCCATCCAGTCTCCCAACTTGTATTAAAGACAGAGGCAGA
AGTTTGGTGGTGGTGGTGTTGGGGTATGGTTTAGTAATATCCACCAGAC
CTTCCGATCCAGCAGTTTGGTGCCCAGAGAGGCATCATGGTGGCTTCCC
TGCGCCCAGGAAGCCATATACACAGATGCCCATTGCAGCATTGTTTGTG
ATAGTGAACAACTGGAAGCTGCTTAACTGTCCATCAGCAGGAGACTGGC
TAAATAAAATTAGAATATATTTATACAACAGAATCTCAAAAACACTGTT
GAGTAAGGAAAAAAAGGCATGCTGCTGAATGATGGGTATGGAACTTTTT
AAAAAAGTACATGCTTTTATGTATGTATATTGCCTATGGATATATGTAT
AAATACAATATGCATCATATATTGATATAACAAGGGTTCTGGAAGGGTA
CACAGAAAACCCACAGCTCGAAGAGTGGTGACGTCTGGGGTGGGGAAGA
AGGGTCTGGGGG
(SEQ ID NO: 33)
MVRLPLQCVLWGCLLTAVHPEPPTACREKQYLINSQCCSLCQPGQKLVS
DCTEFTETECLPCGESEFLDTWNRETHCHQHKYCDPNLGLRVQQKGTSE
TDTICTCEEGWHCTSEACESCVLHRSCSPGFGVKQIATGVSDTICEPCP
VGFFSNVSSAFEKCHPWTSCETKDLVVQQAGTNKTDVVCGPQDRLRALV
VIPIIFGILFAILLVLVFIKKVAKKPTNKAPHPKQEPQEINFPDDLPGS
NTAAPVQETLHGCQPVTQEDGKESRISVQERQ.

Representative nucleotide and amino acid sequences for human CD70 are set forth in SEQ ID NO:34 (accession no. NM_001252) and SEQ ID NO:35, respectively:

(SEQ ID NO: 34)
CCAGAGAGGGGCAGGCTGGTCCCCTGACAGGTTGAAGCAAGTAGACGCC
CAGGAGCCCCGGGAGGGGGCTGCAGTTTCCTTCCTTCCTTCTCGGCAGC
GCTCCGCGCCCCCATCGCCCCTCCTGCGCTAGCGGAGGTGATCGCCGCG
GCGATGCCGGAGGAGGGTTCGGGCTGCTCGGTGCGGCGCAGGCCCTATG
GGTGCGTCCTGCGGGCTGCTTTGGTCCCATTGGTCGCGGGCTTGGTGAT
CTGCCTCGTGGTGTGCATCCAGCGCTTCGCACAGGCTCAGCAGCAGCTG
CCGCTCGAGTCACTTGGGTGGGACGTAGCTGAGCTGCAGCTGAATCACA
CAGGACCTCAGCAGGACCCCAGGCTATACTGGCAGGGGGGCCCAGCACT
GGGCCGCTCCTTCCTGCATGGACCAGAGCTGGACAAGGGGCAGCTACGT
ATCCATCGTGATGGCATCTACATGGTACACATCCAGGTGACGCTGGCCA
TCTGCTCCTCCACGACGGCCTCCAGGCACCACCCCACCACCCTGGCCGT
GGGAATCTGCTCTCCCGCCTCCCGTAGCATCAGCCTGCTGCGTCTCAGC
TTCCACCAAGGTTGTACCATTGCCTCCCAGCGCCTGACGCCCCTGGCCC
GAGGGGACACACTCTGCACCAACCTCACTGGGACACTTTTGCCTTCCCG
AAACACTGATGAGACCTTCTTTGGAGTGCAGTGGGTGCGCCCCTGACCA
CTGCTGCTGATTAGGGTTTTTTAAATTTTATTTTATTTTATTTAAGTTC
AAGAGAAAAAGTGTACACACAGGGGCCACCCGGGGTTGGGGTGGGAGTG
TGGTGGGGGGTAGTGGTGGCAGGACAAGAGAAGGCATTGAGCTTTTTCT
TTCATTTTCCTATTAAAAAATACAAAAATCA
(SEQ ID NO: 35)
MPEEGSGCSVRRRPYGCVLRAALVPLVAGLVICLVVCIQRFAQAQQQLP
LESLGWDVAELQLNHTGPQQDPRLYWQGGPALGRSFLHGPELDKGQLRI
HRDGIYMVHIQVTLAICSSTTASRHHPTTLAVGICSPASRSISLLRLSF
HQGCTIASQRLTPLARGDTLCTNLTGTLLPSRNTDETFFGVQWVRP.

Representative nucleotide and amino acid sequences for human LIGHT are set forth in SEQ ID NO:36 (accession no. CR541854) and SEQ ID NO:37, respectively:

(SEQ ID NO: 36)
ATGGAGGAGAGTGTCGTACGGCCCTCAGTGTTTGTGGTGGATGGACAGA
CCGACATCCCATTCACGAGGCTGGGACGAAGCCACCGGAGACAGTCGTG
CAGTGTGGCCCGGGTGGGTCTGGGTCTCTTGCTGTTGCTGATGGGGGCC
GGGCTGGCCGTCCAAGGCTGGTTCCTCCTGCAGCTGCACTGGCGTCTAG
GAGAGATGGTCACCCGCCTGCCTGACGGACCTGCAGGCTCCTGGGAGCA
GCTGATACAAGAGCGAAGGTCTCACGAGGTCAACCCAGCAGCGCATCTC
ACAGGGGCCAACTCCAGCTTGACCGGCAGCGGGGGGCCGCTGTTATGGG
AGACTCAGCTGGGCCTGGCCTTCCTGAGGGGCCTCAGCTACCACGATGG
GGCCCTTGTGGTCACCAAAGCTGGCTACTACTACATCTACTCCAAGGTG
CAGCTGGGCGGTGTGGGCTGCCCGCTGGGCCTGGCCAGCACCATCACCC
ACGGCCTCTACAAGCGCACACCCCGCTACCCCGAGGAGCTGGAGCTGTT
GGTCAGCCAGCAGTCACCCTGCGGACGGGCCACCAGCAGCTCCCGGGTC
TGGTGGGACAGCAGCTTCCTGGGTGGTGTGGTACACCTGGAGGCTGGGG
AGGAGGTGGTCGTCCGTGTGCTGGATGAACGCCTGGTTCGACTGCGTGA
TGGTACCCGGTCTTACTTCGGGGCTTTCATGGTGTGA
(SEQ ID NO: 37)
MEESWRPSVFVVDGQTDIPFTRLGRSHRRQSCSVARVGLGLLLLLMGAG
LAVQGWFLLQLHWRLGEMVTRLPDGPAGSWEQLIQERRSHEVNPAAHLT
GANSSLTGSGGPLLWETQLGLAFLRGLSYHDGALVVTKAGYYYIYSKVQ
LGGVGCPLGLASTITHGLYKRTPRYPEELELLVSQQSPCGRATSSSRVW
WDSSFLGGWHLEAGEEWVRVLDERLVRLRDGTRSYFGAFMV.

In various embodiments, the present invention provides for variants comprising any of the sequences described herein, for instance, a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with any of the sequences disclosed herein (for example, SEQ ID NOS: 1-13 and 26-37).

In various embodiments, the present invention provides for an amino acid sequence having one or more amino acid mutations relative any of the protein sequences described herein. In some embodiments, the one or more amino acid mutations may be independently selected from conservative or non-conservative substitutions, insertions, deletions, and truncations as described herein.

### Checkpoint Blockade/Blockage of Tumor Immunosuppression

Some human tumors can be eliminated by a patient's immune system. For example, administration of a monoclonal antibody targeted to an immune “checkpoint” molecule can lead to complete response and tumor remission. A mode of action of such antibodies is through inhibition of an immune regulatory molecule that the tumors have co-opted as protection from an anti-tumor immune response. By inhibiting these “checkpoint” molecules (e.g., with an antagonistic antibody), a patient's CD8+ T cells may be allowed to proliferate and destroy tumor cells. For example, administration of a monoclonal antibody targeted to by way of example, without limitation, CTLA-4 or PD-1 can lead to complete response and tumor remission. The mode of action of such antibodies is through inhibition of CTLA-4 or PD-1 that the tumors have co-opted as protection from an anti-tumor immune response. By inhibiting these “checkpoint” molecules (e.g., with an antagonistic antibody), a patient's CD8+ T cells may be allowed to proliferate and destroy tumor cells.

Thus, the cell based therapies provided herein can be used in combination with one or more blocking antibodies targeted to an immune “checkpoint” molecule. For instance, in some embodiments, the cell based therapies provided herein can be used in combination with one or more blocking antibodies targeted to a molecule such as CTLA-4 or PD-1. For example, the cell based therapies provided herein may be used in combination with an agent that blocks, reduces and/or inhibits PD-1 and PD-L1 or PD-L2 and/or the binding of PD-1 with PD-L1 or PD-L2 (by way of non-limiting example, one or more of nivolumab (ONO-4538/BMS-936558, MDX1106, OPDIVO, BRISTOL MYERS SQUIBB), pembrolizumab (KEYTRUDA, Merck), pidilizumab (CT-011, CURE TECH), MK-3475 (MERCK), BMS 936559 (BRISTOL MYERS SQUIBB), MPDL3280A (ROCHE)). In an embodiment, the cell based therapies provided herein may be used in combination with an agent that blocks, reduces and/or inhibits the activity of CTLA-4 and/or the binding of CTLA-4 with one or more receptors (e.g. CD80, CD86, AP2M1, SHP-2, and PPP2R5A). For instance, in some embodiments, the immune-modulating agent is an antibody such as, by way of non-limitation, ipilimumab (MDX-010, MDX-101, Yervoy, BMS) and/or tremelimumab (Pfizer). Blocking antibodies against these molecules can be obtained from, for example, Bristol Myers Squibb (New York, N.Y.), Merck (Kenilworth, N.J.), MedImmune (Gaithersburg, Md.), and Pfizer (New York, N.Y.).

Further, the cell based therapies provided herein can be used in combination with one or more blocking antibodies targeted to an immune “checkpoint” molecule such as for example, BTLA, HVEM, TIM3, GAL9, LAG3, VISTA, KIR, 2B4, CD160 (also referred to as BY55), CGEN-15049, CHK 1 and CHK2 kinases, A2aR, CEACAM (e.g., CEACAM-1, CEACAM-3 and/or CEACAM-5), GITR, GITRL, galectin-9, CD244, CD160, TIGIT, SIRPα, ICOS, CD172a, and TMIGD2 and various B-7 family ligands (including, but are not limited to, B7-1, B7-2, B7-DC, B7-H1, B7-H2, B7-H3, B7-H4, B7-H5, B7-H6 and B7-H7).

### Cell Based Therapies

The present disclosure provides a cell based therapy comprising a first cell containing an expression vector comprising, a nucleotide sequence that encode a secretable vaccine protein (e.g., a gp96-Ig fusion protein) and a second cell containing an expression vector comprising a nucleotide sequence that encodes a T cell costimulatory fusion protein, (e.g., OX40L-Ig or a portion thereof that binds specifically to OX40, ICOSL-Ig or a portion thereof that binds specifically to ICOS, 4-1BBL-Ig, or a portion thereof that binds specifically to 4-1BBR, CD40L-Ig, or a portion thereof that binds specifically to CD40, CD70-Ig, or a portion thereof that binds specifically to CD27, TL1A-Ig or a portion thereof that binds specifically to TNFRSF25, or GITRL-Ig or a portion thereof that binds specifically to GITR). In addition, present disclosure provides methods for making the cell based therapies described herein, as well as methods for administering the cell based therapies. In general, the methods provided herein include administering to a patient an effective amount of a first cell comprising an expression vector comprising, a nucleotide sequence that encodes a secretable vaccine protein, wherein the patient is undergoing a treatment with a second cell comprising an expression vector comprising a nucleotide sequence that encodes a T cell costimulatory fusion protein and wherein the T cell costimulatory fusion protein enhances activation of antigen-specific T cells when administered to the subject.

In some embodiments, the methods provided herein include administering to a patient an effective amount of a second cell comprising an expression vector comprising a nucleotide sequence that encodes a T cell costimulatory fusion protein, wherein the T cell costimulatory fusion protein enhances activation of antigen-specific T cells when administered to the subject, and wherein the patient is undergoing a treatment with a first cell comprising an expression vector comprising, a nucleotide sequence that encodes a secretable vaccine protein.

In some embodiments, the methods provided herein include administering to the patient an effective amount of a first cell comprising an expression vector comprising a nucleotide sequence that encodes a T cell costimulatory fusion protein and a second cell comprising an expression vector comprising a nucleotide sequence that encodes a T cell costimulatory fusion protein and wherein the T cell costimulatory fusion protein enhances activation of antigen-specific T cells when administered to the subject.

In some embodiments, gp96-Ig based vaccines can be generated to stimulate antigen specific immune responses against individual antigens expressed by simian immunodeficiency virus, human immunodeficiency virus, hepatitis C virus and malaria. Immune responses to these vaccines may be enhanced through a cell based therapy of a T cell costimulatory fusion protein and a cell based therapy of gp96-Ig.

cDNA or DNA sequences encoding a vaccine protein fusion (e.g., a gp96-Ig fusion) and a T cell costimulatory fusion protein can be obtained (and, if desired, modified) using conventional DNA cloning and mutagenesis methods, DNA amplification methods, and/or synthetic methods. In general, a sequence encoding a vaccine protein fusion protein (e.g., a gp96-Ig fusion protein) and/or a T cell costimulatory fusion protein can be inserted into a cloning vector for genetic modification and replication purposes prior to expression. Each coding sequence can be operably linked to a regulatory element, such as a promoter, for purposes of expressing the encoded protein in suitable host cells in vitro and in vivo.

The cell based therapy can be administered to produce secreted vaccine proteins (e.g., gp96-Ig) and T cell costimulatory fusion proteins. Cells may be cultured in vitro or genetically engineered, for example. Host cells can be obtained from normal or affected subjects, including healthy humans, cancer patients, and patients with an infectious disease, private laboratory deposits, public culture collections such as the American Type Culture Collection, or from commercial suppliers. Cells that can be used for production and secretion of gp96-Ig fusion proteins and T cell costimulatory fusion proteins in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, or granulocytes, various stem or progenitor cells, such as hematopoietic stem or progenitor cells (e.g., as obtained from bone marrow), umbilical cord blood, peripheral blood, fetal liver, etc., and tumor cells (e.g., human tumor cells). The choice of cell type depends on the type of tumor or infectious disease being treated or prevented, and can be determined by one of skill in the art.

Different host cells have characteristic and specific mechanisms for post-translational processing and modification of proteins. A host cell may be chosen which modifies and processes the expressed gene products in a specific fashion similar to the way the recipient processes its heat shock proteins (hsps). For the purpose of producing large amounts of gp96-Ig, it can be preferable that the type of host cell has been used for expression of heterologous genes, and is reasonably well characterized and developed for large-scale production processes. In some embodiments, the host cells are autologous to the patient to whom the present fusion or recombinant cells secreting the present fusion proteins are subsequently administered.

In some embodiments, the cell based therapy as provided herein can be introduced into an antigenic cell. As used herein, antigenic cells can include preneoplastic cells that are infected with a cancer-causing infectious agent, such as a virus, but that are not yet neoplastic, or antigenic cells that have been exposed to a mutagen or cancer-causing agent, such as a DNA-damaging agent or radiation, for example. Other cells that can be used are preneoplastic cells that are in transition from a normal to a neoplastic form as characterized by morphology or physiological or biochemical function.

Typically, the cancer cells and preneoplastic cells used in the methods provided herein are of mammalian origin. Mammals contemplated include humans, companion animals (e.g., dogs and cats), livestock animals (e.g., sheep, cattle, goats, pigs and horses), laboratory animals (e.g., mice, rats and rabbits), and captive or free wild animals.

In some embodiments, cancer cells (e.g., human tumor cells) can be used in the methods described herein. The cancer cells provide antigenic peptides that become associated non-covalently with the expressed gp96-Ig fusion proteins. Cell lines derived from a preneoplastic lesion, cancer tissue, or cancer cells also can be used, provided that the cells of the cell line have at least one or more antigenic determinant in common with the antigens on the target cancer cells. Cancer tissues, cancer cells, cells infected with a cancer-causing agent, other preneoplastic cells, and cell lines of human origin can be used. Cancer cells excised from the patient to whom ultimately the fusion proteins ultimately are to be administered can be particularly useful, although allogeneic cells also can be used. In some embodiments, a cancer cell can be from an established tumor cell line such as, without limitation, an established non-small cell lung carcinoma (NSCLC), bladder cancer, melanoma, ovarian cancer, renal cell carcinoma, prostate carcinoma, sarcoma, breast carcinoma, squamous cell carcinoma, head and neck carcinoma, hepatocellular carcinoma, pancreatic carcinoma, or colon carcinoma cell line.

In various embodiments, the present fusion proteins allow for both the costimulation T cell and the presentation of various tumor cell antigens. For instance, in some embodiments, the present vaccine protein fusions (e.g., gp96 fusions) chaperone these various tumor antigens. In various embodiments, the tumor cells secrete a variety of antigens. Illustrative, but non-limiting, antigens that can be secreted are: ACRBP, ACTL8, ADAM2, ADAM29, AKAP3, AKAP4, ANKRD45, ARMC3, ARX, ATAD2, BAGE, BAGE2, BAGE3, BAGE4, BAGE5, BRDT, C150RF60, C210RF99, CABYR, CAGE1, CALR3, CASC5, CCDC110, CCDC33, CCDC36, CCDCl62, CCDCl83, CDCA1, CEP290, CEP55, COX6B2, CPXCR1, CRISP2, CSAG1, CSAG2, CSAG3B, CT16.2, CT45A1, CT45A2, CT45A3, CT45A4, CT45A5, CT45A6, CT47A1, CT47A10, CT47A11, CT47A2, CT47A3, CT47A4, CT47A5, CT47A6, CT47A7, CT47A8, CT47A9, CT47B1, CT66, AA884595, CT69, BC040308, CT70, Bl818097, CTAG1A, CTAG1B, CTAG2, CTAGE-2, CTAGE1, CTAGE5, CTCFL, CTNNA2, CXORF48, CXORF61, CYCLIN A1, DCAF12, DDX43, DDX53, DKKL1, DMRT1, DNAJB8, DPPA2, DSCR8, EDAG, NDR, ELOVL4, FAM133A, FAM46D, FATE1, FBX039, FMR1NB, FTHL17, GAGE1, GAGE12B, GAGE12C, GAGE12D, GAGE12E, GAGE12F, GAGE12G, GAGE12H, GAGE12I, GAGE12J, GAGE13, GAGE2A, GAGE3, GAGE4, GAGE5, GAGE6, GAGE7, GAGE8, GOLGAGL2 FA, GPAT2, GPATCH2, HIWI, MIWI, PIWI, HORMAD1, HORMAD2, HSPB9, IGSF11, IL13RA2, IMP-3, JARID1B, KIAA0100, LAGE-1B, LDHC, LEMD1, LIPI, LOC130576, LOC196993, LOC348120, LOC440934, LOC647107, LOC728137, LUZP4, LY6K, MAEL, MAGEA1, MAGEA10, MAGEA11, MAGEA12, MAGEA2, MAGEA2B, MAGEA3, MAGEA4, MAGEA5, MAGEA6, MAGEA8, MAGEA9, MAGEA9B, LOC728269, MAGEB1, MAGEB2, MAGEB3, MAGEB4, MAGEB5, MAGEB6, MAGEC1, MAGEC2, MAGEC3, MCAK, MMA1B, MORC1, MPHOSPH1, NLRP4, NOL4, NR6A1, NXF2, NXF2B, NY-ESO-1, ODF1, ODF2, ODF3, ODF4, OIP5, OTOA, PAGE1, PAGE2, PAGE2B, PAGE3, PAGE4, PAGE5, PASD1, PBK, PEPP2, PIWIL2, PLAC1, POTEA, POTEB, POTEC, POTED, POTEE, POTEG, POTEH, PRAME, PRM1, PRM2, PRSS54, PRSS55, PTPN20A, RBM46, RGS22, ROPN1, RQCD1, SAGE1, SEMG1, SLC06A1, SPA17, SPACA3, SPAG1, SPAG17, SPAG4, SPAG6, SPAG8, SPAG9, SPANXA1, SPANXA2, SPANXB1, SPANXB2, SPANXC, SPANXD, SPANXE, SPANXN1, SPANXN2, SPANXN3, SPANXN4, SPANXN5, SPATA19, SPEF2, SPINLW1, SPO11, SSX1, SSX2, SSX2B, SSX3, SSX4, SSX4B, SSX5, SSX6, SSX7, SSX9, SYCE1, SYCP1, TAF7L, TAG, TDRD1, TDRD4, TDRD6, TEKT5, TEX101, TEX14, TEX15, TFDP3, THEG, TMEFF1, TMEFF2, TMEM108, TMPRSS12, TPPP2, TPTE, TSGA10, TSP50, TSPY1D, TSPY1E, TSPY1F, TSPY1G, TSPY1H, TSPY1I, TSPY2, TSPY3, TSSK6, TTK, TULP2, VENTXP1, XAGE-3B, XAGE-4, RP11-167P23.2, XAGE1, XAGE1B, XAGE1C, XAGE1D, XAGE1E, XAGE2, XAGE2B, CTD-2267G17.3, XAGE3, XAGE5, ZNF165, ZNF645, MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, RAGE, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pme1117, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SCP-1 CT-7, c-erbB-2, CD19, CD20, CD22, CD30, CD33, CD37, CD56, CD70, CD74, CD138, AGS16, MUC1, GPNMB, Ep-CAM, PD-L1, PD-L2, PMSA. In some embodiments, the antigens are human endogenous retroviral antigens. Illustrative antigens can also include antigens from human endogenous retroviruses which include, but are not limited to, epitopes derived from at least a portion of Gag, at least a portion of Tat, at least a portion of Rev, a least a portion of Nef, and at least a portion of gp160.

Further, in some embodiments, the present vaccine protein fusions (e.g., gp96 fusions) provide for an adjuvant effect that further allows the immune system of a patient, when used in the various methods described herein, to be activated against a disease of interest.

Both prokaryotic and eukaryotic vectors can be used for expression of the vaccine protein (e.g., gp96-Ig) and T cell costimulatory fusion proteins in the cell based therapy methods provided herein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and λPL. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful.

A variety of regulatory regions can be used for expression of the vaccine protein (e.g., gp96-Ig) and T cell costimulatory fusions in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the R-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the fusion proteins in recombinant host cells.

In some embodiments, the present invention contemplates the use of inducible promoters capable of effecting high level of expression transiently in response to a cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Animal regulatory regions that exhibit tissue specificity and have been utilized in transgenic animals also can be used in tumor cells of a particular tissue type: the elastase I gene control region that is active in pancreatic acinar cells (Swift et al., Cell 1984, 38:639-646; Ornitz et al., Cold Spring Harbor Symp Quant Biol 1986, 50:399-409; and MacDonald, Hepatology 1987, 7:425-515); the insulin gene control region that is active in pancreatic beta cells (Hanahan, Nature 1985, 315:115-122), the immunoglobulin gene control region that is active in lymphoid cells (Grosschedl et al., Cell 1984, 38:647-658; Adames et al., Nature 1985, 318:533-538; and Alexander et al., Mol Cell Biol 1987, 7:1436-1444), the mouse mammary tumor virus control region that is active in testicular, breast, lymphoid and mast cells (Leder et al., Cell 1986, 45:485-495), the albumin gene control region that is active in liver (Pinkert et al., Genes Devel, 1987, 1:268-276), the alpha-fetoprotein gene control region that is active in liver (Krumlauf et al., Mol Cell Biol 1985, 5:1639-1648; and Hammer et al., Science 1987, 235:53-58); the alpha 1-antitrypsin gene control region that is active in liver (Kelsey et al., Genes Devel 1987, 1:161-171), the beta-globin gene control region that is active in myeloid cells (Mogram et al., Nature 1985, 315:338-340; and Kollias et al., Cell 1986, 46:89-94); the myelin basic protein gene control region that is active in oligodendrocyte cells in the brain (Readhead et al., Cell 1987, 48:703-712); the myosin light chain-2 gene control region that is active in skeletal muscle (Sani, Nature 1985, 314:283-286), and the gonadotropic releasing hormone gene control region that is active in the hypothalamus (Mason et al., Science 1986, 234:1372-1378).

An expression vector also can include transcription enhancer elements, such as those found in SV40 virus, Hepatitis B virus, cytomegalovirus, immunoglobulin genes, metallothionein, and β-actin (see, Bittner et al., Meth Enzymol 1987, 153:516-544; and Gorman, Curr Op Biotechnol 1990, 1:36-47). In addition, an expression vector can contain sequences that permit maintenance and replication of the vector in more than one type of host cell, or integration of the vector into the host chromosome. Such sequences include, without limitation, to replication origins, autonomously replicating sequences (ARS), centromere DNA, and telomere DNA.

In addition, an expression vector can contain one or more selectable or screenable marker genes for initially isolating, identifying, or tracking host cells that contain DNA encoding fusion proteins as described herein. For long term, high yield production of gp96-Ig and T cell costimulatory fusion proteins, stable expression in mammalian cells can be useful. A number of selection systems can be used for mammalian cells. For example, the Herpes simplex virus thymidine kinase (Wigler et al., Cell 1977, 11:223), hypoxanthine-guanine phosphoribosyltransferase (Szybalski and Szybalski, Proc Natl Acad Sci USA 1962, 48:2026), and adenine phosphoribosyltransferase (Lowy et al., Cell 1980, 22:817) genes can be employed in tk−, hgprt−, or aprt− cells, respectively. In addition, antimetabolite resistance can be used as the basis of selection for dihydrofolate reductase (dhfr), which confers resistance to methotrexate (Wigler et al., Proc Natl Acad Sci USA 1980, 77:3567; O'Hare et al., Proc Natl Acad Sci USA 1981, 78:1527); gpt, which confers resistance to mycophenolic acid (Mulligan and Berg, Proc Natl Acad Sci USA 1981, 78:2072); neomycin phosphotransferase (neo), which confers resistance to the aminoglycoside G-418 (Colberre-Garapin et al., J Mol Biol 1981, 150:1); and hygromycin phosphotransferase (hyg), which confers resistance to hygromycin (Santerre et al., Gene 1984, 30:147). Other selectable markers such as histidinol and Zeocin™ also can be used.

Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the fusion proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, human small cell lung carcinoma cell lines, SCLC #2 and SCLC #7, human lung adenocarcinoma cell line, e.g., AD100, and human prostate cancer cell line, e.g., PC-3.

A number of viral-based expression systems also can be used with mammalian cells to produce gp96-Ig and T cell costimulatory fusion proteins. Vectors using DNA virus backbones have been derived from simian virus 40 (SV40) (Hamer et al., Cell 1979, 17:725), adenovirus (Van Doren et al., Mol Cell Biol 1984, 4:1653), adeno-associated virus (McLaughlin et al., J Virol 1988, 62:1963), and bovine papillomas virus (Zinn et al., Proc Natl Acad Sci USA 1982, 79:4897). When an adenovirus is used as an expression vector, the donor DNA sequence may be ligated to an adenovirus transcription/translation control complex, e.g., the late promoter and tripartite leader sequence. This fusion gene may then be inserted in the adenovirus genome by in vitro or in vivo recombination. Insertion in a non-essential region of the viral genome (e.g., region E1 or E3) can result in a recombinant virus that is viable and capable of expressing heterologous products in infected hosts. (See, e.g., Logan and Shenk, Proc Natl Acad Sci USA 1984, 81:3655-3659).

Bovine papillomavirus (BPV) can infect many higher vertebrates, including man, and its DNA replicates as an episome. A number of shuttle vectors have been developed for recombinant gene expression which exist as stable, multicopy (20-300 copies/cell) extrachromosomal elements in mammalian cells. Typically, these vectors contain a segment of BPV DNA (the entire genome or a 69% transforming fragment), a promoter with a broad host range, a polyadenylation signal, splice signals, a selectable marker, and “poisonless” plasmid sequences that allow the vector to be propagated in E. coli. Following construction and amplification in bacteria, the expression gene constructs are transfected into cultured mammalian cells by, for example, calcium phosphate coprecipitation. For those host cells that do not manifest a transformed phenotype, selection of transformants is achieved by use of a dominant selectable marker, such as histidinol and G418 resistance.

Alternatively, the vaccinia7.5K promoter can be used. (See, e.g., Mackett et al., Proc Natl Acad Sci USA 1982, 79:7415-7419; Mackett et al., J Virol 1984, 49:857-864; and Panicali et al., Proc Natl Acad Sci USA 1982, 79:4927-4931.) In cases where a human host cell is used, vectors based on the Epstein-Barr virus (EBV) origin (OriP) and EBV nuclear antigen 1 (EBNA-1; a trans-acting replication factor) can be used. Such vectors can be used with a broad range of human host cells, e.g., EBO-pCD (Spickofsky et al., DNA Prot Eng Tech 1990, 2:14-18); pDR2 and ADR2 (available from Clontech Laboratories).

Gp96-Ig and T cell costimulatory fusion proteins also can be made with retrovirus-based expression systems. Retroviruses, such as Moloney murine leukemia virus, can be used since most of the viral gene sequence can be removed and replaced with exogenous coding sequence while the missing viral functions can be supplied in trans. In contrast to transfection, retroviruses can efficiently infect and transfer genes to a wide range of cell types including, for example, primary hematopoietic cells. Moreover, the host range for infection by a retroviral vector can be manipulated by the choice of envelope used for vector packaging.

For example, a retroviral vector can comprise a 5′ long terminal repeat (LTR), a 3′ LTR, a packaging signal, a bacterial origin of replication, and a selectable marker. The gp96-Ig fusion protein coding sequence, for example, can be inserted into a position between the 5′ LTR and 3′ LTR, such that transcription from the 5′ LTR promoter transcribes the cloned DNA. The 5′ LTR contains a promoter (e.g., an LTR promoter), an R region, a U5 region, and a primer binding site, in that order. Nucleotide sequences of these LTR elements are well known in the art. A heterologous promoter as well as multiple drug selection markers also can be included in the expression vector to facilitate selection of infected cells. See, McLauchlin et al., Prog Nucleic Acid Res Mol Biol 1990, 38:91-135; Morgenstern et al., Nucleic Acid Res 1990, 18:3587-3596; Choulika et al., J Virol 1996, 70:1792-1798; Boesen et al., Biotherapy 1994, 6:291-302; Salmons and Gunzberg, Human Gene Ther 1993, 4:129-141; and Grossman and Wilson, Curr Opin Genet Devel 1993, 3:110-114.

Any of the cloning and expression vectors described herein may be synthesized and assembled from known DNA sequences using techniques that are known in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

### Methods of Treating

Cell based therapies can be used in administration to a subject (e.g., a research animal or a mammal, such as a human, having a clinical condition such as cancer or an infection). For example, the cell based therapy can be administered to a subject for the treatment of cancer or infection. Thus, this document provides methods for treating clinical conditions such as cancer or infection with the expression vectors provided herein. The infection can be, for example, an acute infection or a chronic infection. In some embodiments, the infection can be an infection by hepatitis C virus, hepatitis B virus, human immunodeficiency virus, or malaria. The methods can include administering to a subject the cell based therapies under conditions wherein the progression or a symptom of the clinical condition in the subject is reduced in a therapeutic manner.

In various embodiments, the present invention pertains to cancers and/or tumors; for example, the treatment or prevention of cancers and/or tumors. Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be lymphoma. The cancer may invade a tissue such as liver, lung, bladder, or intestinal.

Illustrative cancers that may be treated include, but are not limited to, carcinomas, e.g. various subtypes, including, for example, adenocarcinoma, basal cell carcinoma, squamous cell carcinoma, and transitional cell carcinoma), sarcomas (including, for example, bone and soft tissue), leukemias (including, for example, acute myeloid, acute lymphoblastic, chronic myeloid, chronic lymphocytic, and hairy cell), lymphomas and myelomas (including, for example, Hodgkin and non-Hodgkin lymphomas, light chain, non-secretory, MGUS, and plasmacytomas), and central nervous system cancers (including, for example, brain (e.g. gliomas (e.g., astrocytoma, oligodendroglioma, and ependymoma), meningioma, pituitary adenoma, and neuromas, and spinal cord tumors (e.g., meningiomas and neurofibroma).

Representative cancers and/or tumors of the present invention include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some aspects, the present fusions are used to eliminate intracellular pathogens. In some aspects, the present fusions are used to treat one or more infections. In some embodiments, the present fusion proteins are used in methods of treating viral infections (including, for example, HIV and HCV), parasitic infections (including, for example, malaria), and bacterial infections. In various embodiments, the infections induce immunosuppression. For example, HIV infections often result in immunosuppression in the infected subjects. Accordingly, as described elsewhere herein, the treatment of such infections may involve, in various embodiments, modulating the immune system with the present fusion proteins to favor immune stimulation over immune inhibition. Alternatively, the present invention provides methods for treating infections that induce immunoactivation. For example, intestinal helminth infections have been associated with chronic immune activation. In these embodiments, the treatment of such infections may involve modulating the immune system with the present fusion proteins to favor immune inhibition over immune stimulation.

In various embodiments, the present invention provides methods of treating viral infections including, without limitation, acute or chronic viral infections, for example, of the respiratory tract, of papilloma virus infections, of herpes simplex virus (HSV) infection, of human immunodeficiency virus (HIV) infection, and of viral infection of internal organs such as infection with hepatitis viruses. In some embodiments, the viral infection is caused by a virus of family Flaviviridae. In some embodiments, the virus of family Flaviviridae is selected from Yellow Fever Virus, West Nile virus, Dengue virus, Japanese Encephalitis Virus, St. Louis Encephalitis Virus, and Hepatitis C Virus. In other embodiments, the viral infection is caused by a virus of family Picornaviridae, e.g., poliovirus, rhinovirus, coxsackievirus. In other embodiments, the viral infection is caused by a member of Orthomyxoviridae, e.g., an influenza virus. In other embodiments, the viral infection is caused by a member of Retroviridae, e.g., a lentivirus. In other embodiments, the viral infection is caused by a member of Paramyxoviridae, e.g., respiratory syncytial virus, a human parainfluenza virus, rubulavirus (e.g., mumps virus), measles virus, and human metapneumovirus. In other embodiments, the viral infection is caused by a member of Bunyaviridae, e.g., hantavirus. In other embodiments, the viral infection is caused by a member of Reoviridae, e.g., a rotavirus.

In various embodiments, the present invention provides methods of treating parasitic infections such as protozoan or helminths infections. In some embodiments, the parasitic infection is by a protozoan parasite. In some embodiments, the oritiziab parasite is selected from intestinal protozoa, tissue protozoa, or blood protozoa. Illustrative protozoan parasites include, but are not limited to, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falciparum, Trichomonas vaginalis, and Histomonas meleagridis. In some embodiments, the parasitic infection is by a helminthic parasite such as nematodes (e.g., Adenophorea). In some embodiments, the parasite is selected from Secementea (e.g., Trichuris trichiura, Ascaris lumbricoides, Enterobius vermicularis, Ancylostoma duodenale, Necator americanus, Strongyloides stercoralis, Wuchereria bancrofti, Dracunculus medinensis). In some embodiments, the parasite is selected from trematodes (e.g. blood flukes, liver flukes, intestinal flukes, and lung flukes). In some embodiments, the parasite is selected from: Schistosoma mansoni, Schistosoma haematobium, Schistosoma japonicum, Fasciola hepatica, Fasciola gigantica, Heterophyes heterophyes, Paragonimus westermani. In some embodiments, the parasite is selected from cestodes (e.g., Taenia solium, Taenia saginata, Hymenolepis nana, Echinococcus granulosus).

In various embodiments, the present invention provides methods of treating bacterial infections. In various embodiments, the bacterial infection is by a gram-positive bacterium, gram-negative bacteria, aerobic and/or anaerobic bacteria. In various embodiments, the bacteria is selected from, but not limited to, Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Nisseria, Baccillus, Bacteroides, Peptococcus, Clostridium, Salmonella, Shigella, Serratia, Haemophilus, Brucella and other organisms. In some embodiments, the bacteria is selected from, but not limited to, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus. The cell based therapy to be administered can be admixed, encapsulated, conjugated or otherwise associated with other molecules, molecular structures, or mixtures of compounds such as, for example, liposomes, receptor or cell targeted molecules, or oral, topical or other formulations for assisting in uptake, distribution and/or absorption. The cell based therapy to be administered can be in combination with a pharmaceutically acceptable carrier.

This present disclosure therefore also provides compositions containing cell based therapies described herein, in combination with a physiologically and pharmaceutically acceptable carrier. The physiologically and pharmaceutically acceptable carrier can be include any of the well-known components useful for immunization. The carrier can facilitate or enhance an immune response to an antigen administered in a vaccine. The cell formulations can contain buffers to maintain a preferred pH range, salts or other components that present an antigen to an individual in a composition that stimulates an immune response to the antigen. The physiologically acceptable carrier also can contain one or more adjuvants that enhance the immune response to an antigen. Pharmaceutically acceptable carriers include, for example, pharmaceutically acceptable solvents, suspending agents, or any other pharmacologically inert vehicles for delivering compounds to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water, saline solution, binding agents (e.g., polyvinylpyrrolidone or hydroxypropyl methylcellulose); fillers (e.g., lactose or dextrose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). Compositions can be formulated for subcutaneous, intramuscular, or intradermal administration, or in any manner acceptable for immunization.

An adjuvant refers to a substance which, when added to an immunogenic agent such as a tumor cell expressing secreted vaccine protein (e.g., gp96-Ig) and T cell costimulatory fusion polypeptides, nonspecifically enhances or potentiates an immune response to the agent in the recipient host upon exposure to the mixture. Adjuvants can include, for example, oil-in-water emulsions, water-in oil emulsions, alum (aluminum salts), liposomes and microparticles, such as, polysytrene, starch, polyphosphazene and polylactide/polyglycosides.

Adjuvants can also include, for example, squalene mixtures (SAF-I), muramyl peptide, saponin derivatives, mycobacterium cell wall preparations, monophosphoryl lipid A, mycolic acid derivatives, nonionic block copolymer surfactants, Quil A, cholera toxin B subunit, polyphosphazene and derivatives, and immunostimulating complexes (ISCOMs) such as those described by Takahashi et al., Nature 1990, 344:873-875. For veterinary use and for production of antibodies in animals, mitogenic components of Freund's adjuvant (both complete and incomplete) can be used. In humans, Incomplete Freund's Adjuvant (IFA) is a useful adjuvant. Various appropriate adjuvants are well known in the art (see, for example, Warren and Chedid, CRC Critical Reviews in Immunology 1988, 8:83; and Allison and Byars, in Vaccines: New Approaches to Immunological Problems, 1992, Ellis, ed., Butterworth-Heinemann, Boston). Additional adjuvants include, for example, bacille Calmett-Guerin (BCG), DETOX (containing cell wall skeleton of Mycobacterium phlei (CWS) and monophosphoryl lipid A from Salmonella minnesota (MPL)), and the like (see, for example, Hoover et al., J Clin Oncol 1993, 11:390; and Woodlock et al., J Immunother 1999, 22:251-259).

In some embodiments, a cell secreting a T cell costimulatory fusion protein (e.g., OX40L-Ig) can be administered to a subject at about 100,000 cells, about 150,000 cells, about 200,000 cells, about 250,000 cells, about 300,000 cells, about 350,000 cells, about 400,000 cells, about 450,000 cells, about 500,000 cells, about 550,000 cells, about 600,000 cells, about 650,000 cells, about 700,000 cells, about 750,000 cells, about 800,000 cells, about 850,000 cells, about 900,000 cells, about 950,000 cells, about 1 million cells, about 1.5 million cells, about 2 million cells, about 2.5 million cells, about 3 million cells, about 3.5 million cells, about 4 million cells, about 4.5 million cells, about 6 million cells, about 6.5 million cells, about 7 million cells, about 7.5 million cells, about 8 million cells, about 8.5 million cells, about 9 million cells, about 9.5 million cells, or about 10 million cells.

In some embodiments, a cell secreting a vaccine protein (e.g., gp96-Ig) can be administered to a subject at about 100,000 cells, about 150,000 cells, about 200,000 cells, about 250,000 cells, about 300,000 cells, about 350,000 cells, about 400,000 cells, about 450,000 cells, about 500,000 cells, about 550,000 cells, about 600,000 cells, about 650,000 cells, about 700,000 cells, about 750,000 cells, about 800,000 cells, about 850,000 cells, about 900,000 cells, about 950,000 cells, about 1 million cells, about 1.5 million cells, about 2 million cells, about 2.5 million cells, about 3 million cells, about 3.5 million cells, about 4 million cells, about 4.5 million cells, about 6 million cells, about 6.5 million cells, about 7 million cells, about 7.5 million cells, about 8 million cells, about 8.5 million cells, about 9 million cells, about 9.5 million cells, or about 10 million cells.

In some embodiments, a fixed dose of a cell secreting a vaccine protein is 1×10⁷ cells.

In some embodiments, a fixed dose of a cell secreting a T cell costimulatory fusion protein (e.g., OX40L-Ig) is 1×10⁷ cells.

In some embodiments, about a cell secreting a T cell costimulatory fusion protein (e.g., OX40L-Ig) can be administered to a subject at about 0.1-1.1×10⁷ cells, about 0.2-1.1×10⁷ cells, about 0.3-1.1×10⁷ cells, about 0.4-1.1×10⁷ cells, about 0.5-1.1×10⁷ cells, about 0.6-1.1×10⁷ cells, about 0.7-1.1×10⁷ cells, about 0.8-1.1×10⁷ cells, about or 0.9-1.1×10⁷ cells, about 0.1-2.1×10⁷ cells, about 0.2-2.1×10⁷ cells, about 0.3-2.1×10⁷ cells, about 0.4-2.1×10⁷ cells, about 0.5-2.1×10⁷ cells, about 0.6-2.1×10⁷ cells, about 0.7-2.1×10⁷ cells, about 0.8-2.1×10⁷ cells, about 0.9-2.1×10⁷ cells, about 0.1-3.1×10⁷ cells, about 0.2-3.1×10⁷ cells, about 0.3-3.1×10⁷ cells, about 0.4-3.1×10⁷ cells, about 0.5-3.1×10⁷ cells, about 0.6-3.1×10⁷ cells, about 0.7-3.1×10⁷ cells, about 0.8-3.1×10⁷ cells, about 0.9-3.1×10⁷ cells, about 0.1-4.1×10⁷ cells, about 0.2-4.1×10⁷ cells, about 0.3-4.1×10⁷ cells, about 0.4-4.1×10⁷ cells, about 0.5-4.1×10⁷ cells, about 0.6-4.1×10⁷ cells, about 0.7-4.1×10⁷ cells, about 0.8-4.1×10⁷ cells, about 0.9-4.1×10⁷ cells, about 0.1-5.1×10⁷ cells, about 0.2-5.1×10⁷ cells, about 0.3-5.1×10⁷ cells, about 0.4-5.1×10⁷ cells, about 0.5-5.1×10⁷ cells, about 0.6-5.1×10⁷ cells, about 0.7-5.1×10⁷ cells, about 0.8-5.1×10⁷ cells, about 0.9-5.1×10⁷ cells, about 0.1-6.1×10⁷ cells, about 0.2-6.1×10⁷ cells, about 0.3-6.1×10⁷ cells, about 0.4-6.1×10⁷ cells, about 0.5-6.1×10⁷ cells, about 0.6-6.1×10⁷ cells, about 0.7-6.1×10⁷ cells, about 0.8-6.1×10⁷ cells, about 0.9-6.1×10⁷ cells, about 0.1-7.1×10⁷ cells, about 0.2-7.1×10⁷ cells, about 0.3-7.1×10⁷ cells, about 0.4-7.1×10⁷ cells, about 0.5-7.1×10⁷ cells, about 0.6-7.1×10⁷ cells, about 0.7-7.1×10⁷ cells, about 0.8-7.1×10⁷ cells, about 0.9-7.1×10⁷ cells, about 0.1-8.1×10⁷ cells, about 0.2-8.1×10⁷ cells, about 0.3-8.1×10⁷ cells, about 0.4-8.1×10⁷ cells, about 0.5-8.1×10⁷ cells, about 0.6-8.1×10⁷ cells, about 0.7-8.1×10⁷ cells, about 0.8-8.1×10⁷ cells, about 0.9-8.1×10⁷ cells, about 0.1-9.1×10⁷ cells, about 0.2-9.1×10⁷ cells, about 0.3-9.1×10⁷ cells, about 0.4-9.1×10⁷ cells, about 0.5-9.1×10⁷ cells, about 0.6-9.1×10⁷ cells, about 0.7-9.1×10⁷ cells, about 0.8-9.1×10⁷ cells, or about 0.9-9.1×10⁷ cells.

In some embodiments, a cell secreting a vaccine protein (e.g., gp96-Ig) can be administered to a subject at about 0.1-1.1×10⁷ cells, about 0.2-1.1×10⁷ cells, about 0.3-1.1×10⁷ cells, about 0.4-1.1×10⁷ cells, about 0.5-1.1×10⁷ cells, about 0.6-1.1×10⁷ cells, about 0.7-1.1×10⁷ cells, about 0.8-1.1×10⁷ cells, about or 0.9-1.1×10⁷ cells, about 0.1-2.1×10⁷ cells, about 0.2-2.1×10⁷ cells, about 0.3-2.1×10⁷ cells, about 0.4-2.1×10⁷ cells, about 0.5-2.1×10⁷ cells, about 0.6-2.1×10⁷ cells, about 0.7-2.1×10⁷ cells, about 0.8-2.1×10⁷ cells, about 0.9-2.1×10⁷ cells, about 0.1-3.1×10⁷ cells, about 0.2-3.1×10⁷ cells, about 0.3-3.1×10⁷ cells, about 0.4-3.1×10⁷ cells, about 0.5-3.1×10⁷ cells, about 0.6-3.1×10⁷ cells, about 0.7-3.1×10⁷ cells, about 0.8-3.1×10⁷ cells, about 0.9-3.1×10⁷ cells, about 0.1-4.1×10⁷ cells, about 0.2-4.1×10⁷ cells, about 0.3-4.1×10⁷ cells, about 0.4-4.1×10⁷ cells, about 0.5-4.1×10⁷ cells, about 0.6-4.1×10⁷ cells, about 0.7-4.1×10⁷ cells, about 0.8-4.1×10⁷ cells, about 0.9-4.1×10⁷ cells, about 0.1-5.1×10⁷ cells, about 0.2-5.1×10⁷ cells, about 0.3-5.1×10⁷ cells, about 0.4-5.1×10⁷ cells, about 0.5-5.1×10⁷ cells, about 0.6-5.1×10⁷ cells, about 0.7-5.1×10⁷ cells, about 0.8-5.1×10⁷ cells, about 0.9-5.1×10⁷ cells, about 0.1-6.1×10⁷ cells, about 0.2-6.1×10⁷ cells, about 0.3-6.1×10⁷ cells, about 0.4-6.1×10⁷ cells, about 0.5-6.1×10⁷ cells, about 0.6-6.1×10⁷ cells, about 0.7-6.1×10⁷ cells, about 0.8-6.1×10⁷ cells, about 0.9-6.1×10⁷ cells, about 0.1-7.1×10⁷ cells, about 0.2-7.1×10⁷ cells, about 0.3-7.1×10⁷ cells, about 0.4-7.1×10⁷ cells, about 0.5-7.1×10⁷ cells, about 0.6-7.1×10⁷ cells, about 0.7-7.1×10⁷ cells, about 0.8-7.1×10⁷ cells, about 0.9-7.1×10⁷ cells, about 0.1-8.1×10⁷ cells, about 0.2-8.1×10⁷ cells, about 0.3-8.1×10⁷ cells, about 0.4-8.1×10⁷ cells, about 0.5-8.1×10⁷ cells, about 0.6-8.1×10⁷ cells, about 0.7-8.1×10⁷ cells, about 0.8-8.1×10⁷ cells, about 0.9-8.1×10⁷ cells, about 0.1-9.1×10⁷ cells, about 0.2-9.1×10⁷ cells, about 0.3-9.1×10⁷ cells, about 0.4-9.1×10⁷ cells, about 0.5-9.1×10⁷ cells, about 0.6-9.1×10⁷ cells, about 0.7-9.1×10⁷ cells, about 0.8-9.1×10⁷ cells, or about 0.9-9.1×10⁷ cells.

In some embodiments, the cell based therapy can be administered to a subject one or more times (e.g., once, twice, two to four times, three to five times, five to eight times, six to ten times, eight to 12 times, or more than 12 times). The cell based therapy as provided herein can be administered one or more times per day, one or more times per week, every other week, one or more times per month, once every two to three months, once every three to six months, or once every six to 12 months. The cell based therapy can be administered over any suitable period of time, such as a period from about 1 day to about 12 months. In some embodiments, for example, the period of administration can be from about 1 day to 90 days; from about 1 day to 60 days; from about 1 day to 30 days; from about 1 day to 20 days; from about 1 day to 10 days; from about 1 day to 7 days. In some embodiments, the period of administration can be from about 1 week to 50 weeks; from about 1 week to 50 weeks; from about 1 week to 40 weeks; from about 1 week to 30 weeks; from about 1 week to 24 weeks; from about 1 week to 20 weeks; from about 1 week to 16 weeks; from about 1 week to 12 weeks; from about 1 week to 8 weeks; from about 1 week to 4 weeks; from about 1 week to 3 weeks; from about 1 week to 2 weeks; from about 2 weeks to 3 weeks; from about 2 weeks to 4 weeks; from about 2 weeks to 6 weeks; from about 2 weeks to 8 weeks; from about 3 weeks to 8 weeks; from about 3 weeks to 12 weeks; or from about 4 weeks to 20 weeks or any weekly increment of time in between.

In some embodiments, after an initial dose (sometimes referred to as a “priming” dose) of a cell based therapy has been administered and a maximal antigen-specific immune response has been achieved, one or more boosting doses of the cell based therapy as provided herein can be administered. For example, a boosting dose can be administered about 10 to 30 days, about 15 to 35 days, about 20 to 40 days, about 25 to 45 days, or about 30 to 50 days after a priming dose.

In some embodiments, a secretable vaccine protein (e.g., gp96-Ig) and the T cell costimulatory fusion protein (e.g., a cell secreting OX40L-Ig), are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

In some embodiments, a regimen is provided in which a first treatment of a cell secreting a T cell costimulatory fusion protein (e.g., OX40L-Ig) is administered and subsequently a second treatment of a cell secreting a T cell costimulatory fusion protein (e.g., OX40L-Ig) is administered and a cell secreting a vaccine protein (e.g., gp96-Ig) is administered. For instance, in some embodiments, the first treatment and the second treatment are about 3 days, or about 5 days, or about 1 week, or about 2 weeks or about 3 weeks apart.

In some embodiments, the first and second treatments are about two weeks apart.

In some embodiments, a single fixed dose of a cell secreting a T cell costimulatory fusion protein (e.g., OX40L-Ig) is administered and following the first dose of the cell secreting the T cell costimulatory fusion protein (e.g., OX40L-Ig), a second dose is administered along with a fixed dose of a cell secreting a vaccine protein (e.g., gp96-Ig) is administered.

In some embodiments, a single fixed dose of a cell secreting the secretable vaccine protein (e.g., gp96-Ig) is administered with an ascending dose of the cell secreting the T cell costimulatory fusion protein (e.g., OX40L-Ig).

In some embodiments, the methods provided herein can be used for controlling solid tumor growth (e.g., breast, prostate, melanoma, renal, colon, or cervical tumor growth) and/or metastasis. The methods can include administering an effective amount of a cell based therapy as described herein to a subject in need thereof. In some embodiments, the subject is a mammal (e.g., a human).

The cell based therapies and methods provided herein can be useful for stimulating an immune response against a tumor. Such immune response is useful in treating or alleviating a sign or symptom associated with the tumor. As used herein, by “treating” is meant reducing, preventing, and/or reversing the symptoms in the individual to which a cell based therapy as described herein has been administered, as compared to the symptoms of an individual not being treated. A practitioner will appreciate that the methods described herein are to be used in concomitance with continuous clinical evaluations by a skilled practitioner (physician or veterinarian) to determine subsequent therapy. Such evaluations will aid and inform in evaluating whether to increase, reduce, or continue a particular treatment dose, mode of administration, etc.

The methods provided herein can thus be used to treat a tumor, including, for example, a cancer. The methods can be used, for example, to inhibit the growth of a tumor by preventing further tumor growth, by slowing tumor growth, or by causing tumor regression. Thus, the methods can be used, for example, to treat a cancer such as a lung cancer. It will be understood that the subject to which a compound is administered need not suffer from a specific traumatic state. Indeed, the cell based therapy described herein may be administered prophylactically, prior to development of symptoms (e.g., a patient in remission from cancer). The terms “therapeutic” and “therapeutically,” and permutations of these terms, are used to encompass therapeutic, palliative, and prophylactic uses. Thus, as used herein, by “treating or alleviating the symptoms” is meant reducing, preventing, and/or reversing the symptoms of the individual to which a therapeutically effective amount of a composition has been administered, as compared to the symptoms of an individual receiving no such administration.

As used herein, the terms “effective amount” and “therapeutically effective amount” refer to an amount sufficient to provide the desired therapeutic (e.g., anti-cancer, anti-tumor, or anti-infection) effect in a subject (e.g., a human diagnosed as having cancer or an infection). Anti-tumor and anti-cancer effects include, without limitation, modulation of tumor growth (e.g., tumor growth delay), tumor size, or metastasis, the reduction of toxicity and side effects associated with a particular anti-cancer agent, the amelioration or minimization of the clinical impairment or symptoms of cancer, extending the survival of the subject beyond that which would otherwise be expected in the absence of such treatment, and the prevention of tumor growth in an animal lacking tumor formation prior to administration, i.e., prophylactic administration. In some embodiments, administration of an effective amount of the cell based therapy can increase the activation or proliferation of tumor antigen specific T cells in a subject. For example, the activation or proliferation of tumor antigen specific T cells in the subject can be is increased by at least 10 percent (e.g., at least 25 percent, at least 50 percent, or at least 75 percent) as compared to the level of activation or proliferation of tumor antigen specific T cells in the subject prior to the administration.

Anti-infection effects include, for example, a reduction in the number of infective agents (e.g., viruses or bacteria). When the clinical condition in the subject to be treated is an infection, administration of a cell based therapy as provided herein can stimulate the activation or proliferation of pathogenic antigen specific T cells in the subject. For example, administration of the cell based therapy can lead to activation of antigen-specific T cells in the subject to a level great than that achieved by gp96-Ig vaccination alone.

One of skill will appreciate that an effective amount of a cell based therapy may be lowered or increased by fine tuning and/or by administering more than one dose (e.g., by concomitant administration of two different genetically modified tumor cells, or by administering the cell based therapy with another agent (e.g., an antagonist of PD-1) to enhance the therapeutic effect (e.g., synergistically). This disclosure therefore provides a method for tailoring the administration/treatment to the particular exigencies specific to a given mammal. Therapeutically effective amounts can be determined by, for example, starting at relatively low amounts and using step-wise increments with concurrent evaluation of beneficial effects. The methods provided herein thus can be used alone or in combination with other well-known tumor therapies, to treat a patient having a tumor. One skilled in the art will readily understand advantageous uses of the cell based therapies and methods provided herein, for example, in prolonging the life expectancy of a cancer patient and/or improving the quality of life of a cancer patient (e.g., a lung cancer patient).

### Combination Therapies and Conjugation

In some embodiments, the invention provides for methods that further comprise administering an additional agent to a subject. In some embodiments, the invention pertains to co-administration and/or co-formulation.

In some embodiments, administration of a first cell comprising an expression vector comprising, a nucleotide sequence that encodes a secretable vaccine protein, to a patient undergoing a treatment with a second cell comprising an expression vector comprising a nucleotide sequence that encodes a T cell costimulatory fusion protein act synergistically when co-administered with another agent.

In some embodiments, administration of vaccine protein (e.g., gp96-Ig) and one or more costimulatory molecules act synergistically when co-administered with another agent and is administered at doses that are lower than the doses commonly employed when such agents are used as monotherapy.

In some embodiments, inclusive of, without limitation, cancer applications, the present invention pertains to chemotherapeutic agents as additional agents. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In some embodiments, inclusive of, without limitation, infectious disease applications, the present invention pertains to anti-infectives as additional agents. In some embodiments, the anti-infective is an anti-viral agent including, but not limited to, Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, and Foscarnet. In some embodiments, the anti-infective is an anti-bacterial agent including, but not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In some embodiments, the anti-infectives include anti-malarial agents (e.g., chloroquine, quinine, mefloquine, primaquine, doxycycline, artemether/lumefantrine, atovaquone/proguanil and sulfadoxine/pyrimethamine), metronidazole, tinidazole, ivermectin, pyrantel pamoate, and albendazole.

Other additional agents are described elsewhere herein, including the blocking antibodies targeted to an immune “checkpoint” molecules.

In some embodiments, the method comprises administration in combination with an agent that inhibits immunosuppressive molecules produced by tumor cells. In some embodiments, the agent is an antibody against PD-1. In some embodiments, the antibody against PD-1 is selected from nivolumab, pembrolizumab, pidilizumab, cemiplimab, AGEN2034, AMP-224, AMP-514, and PDR001.

In some embodiments, the agent is an antibody against PD-L1. In some embodiments, the antibody against PD-L1 is selected from Atezolizumab (Tecentriq), Avelumab (Bavencio), Durvalumab (Imfinzi), BMS-936559, and CK-301.

In some embodiments, the agent is an antibody against CTLA-4. In some embodiments, the antibody against CTLA-4 is selected from ipilimumab, tremelimumab, AGEN1884, and RG2077.

In some embodiments, the agent is an antibody against OX40. In some embodiments, the antibody against OX40 is selected from PF-04518600, BMS-986178, INCAGN01949, MEDI0562, GSK1795091, and GSK3174998 Subjects and/or Animals

The methods described herein are intended for use with any subject that may experience the benefits of these methods. Thus, “subjects,” “patients,” and “individuals” (used interchangeably) include humans as well as non-human subjects, particularly domesticated animals.

In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g., GFP). In some embodiments, the subject and/or animal is a transgenic animal comprising a fluorescent cell.

In some embodiments, the subject and/or animal is a human. In some embodiments, the human is a pediatric human. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the non-human animal is a livestock animal. In certain embodiments, the subject is a human cancer patient that cannot receive chemotherapy, e.g., the patient is unresponsive to chemotherapy or too ill to have a suitable therapeutic window for chemotherapy (e.g., experiencing too many dose- or regimen-limiting side effects). In certain embodiments, the subject is a human cancer patient having advanced and/or metastatic disease.

As used herein, an “allogeneic cell” refers to a cell that is not derived from the individual to which the cell is to be administered, that is, has a different genetic constitution than the individual. An allogeneic cell is generally obtained from the same species as the individual to which the cell is to be administered. For example, the allogeneic cell can be a human cell, as disclosed herein, for administering to a human patient such as a cancer patient. As used herein, an “allogeneic tumor cell” refers to a tumor cell that is not derived from the individual to which the allogeneic cell is to be administered. Generally, the allogeneic tumor cell expresses one or more tumor antigens that can stimulate an immune response against a tumor in an individual to which the cell is to be administered. As used herein, an “allogeneic cancer cell,” for example, a lung cancer cell, refers to a cancer cell that is not derived from the individual to which the allogeneic cell is to be administered.

As used herein, a “genetically modified cell” refers to a cell that has been genetically modified to express an exogenous nucleic acid, for example, by transfection or transduction.

Technical and scientific terms used herein have the meaning commonly understood by one of skill in the art to which the present invention pertains, unless otherwise defined.

As used herein, the singular forms “a,” “an” and “the” specifically also encompass the plural forms of the terms to which they refer, unless the content clearly dictates otherwise. As used herein, unless specifically indicated otherwise, the word “or” is used in the “inclusive” sense of “and/or” and not the “exclusive” sense of either/or.” In the specification and the appended claims, the singular forms include plural referents unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. As used in this specification, whether in a transitional phrase or in the body of the claim, the terms “comprise (s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components. The invention will be further described in the following examples, which do not limit the scope of the invention described in the claims.

### EXAMPLES

In order that the invention disclosed herein may be more efficiently understood, examples are provided below. It should be understood that these examples are for illustrative purposes only and are not to be construed as limiting the invention in any manner.

### Example 1: Planned Dose Range Finding Studies in Mice to Support Clinical Dosing

The following nonclinical study is conducted in mice using a surrogate (specific for animal species to be tested) cell-based vaccine for HS-130, which is a genetically engineered human lung adenocarcinoma cell line secreting OX40L-Ig fusion protein, also (referred to as mHS-130 (B16F10) and HS-110 (referred to as mHS-110 (B16F10), generated from a B16F10-ova cell line, to determine a dose of mHS-130 when co-administered with a fixed dose of mHS-110. The starting dose of HS-130 in humans is based on the dose of mHS-130 that generates a minimum response (MABEL) in the mouse model based on the proportional use of mHS-110 in mice vs. the current dose of HS-110 in humans.

In previous studies, the dose of murine HS-110 (mHS-110) in mice was determined to be 1×10⁶ cells. It was further determined by quantitative ELISA that this number of cells secrete 290 ng of gp96-Ig fusion protein per 24 hours. In order to determine the ratio of gp96-Ig to OX40L-Ig in this system, a murine form of HS-130 [mHS-130 (B16F10)] secreting the species specific OX40L-Ig protein is generated. It has also been determined by quantitative ELISA that mHS-130 (B16F10) secretes 850 ng of OX40L-Ig per 10⁶ cells per 24 hours.

### Experimental Design

Wild-type C57BL/6 mice are adoptively transferred with 10⁶ OT-I^{gfp} and 10⁶ OT-II^{FoxP3-rfp} congenic cells. These cells are transgenic CD8+ and CD4+ T cells designed to recognize a specific ovalbumin peptide. When transferred into wild-type mice these cells join the other murine lymphocytes in circulation until they encounter their cognate antigen and can be activated. Both of the murine vaccine cell lines mHS-110 (B16F10) and mHS-130 (B16F10) are engineered to produce ovalbumin, a protein not naturally expressed in the mouse. Two days after adoptive transfer the mice are vaccinated with mHS-110 (B16F10) and mHS-130 (B16F10) according to the doses described in the study protocol below (Table 1). Mice are followed for 14 days. Periodically, peripheral blood is collected from the tail vein and analyzed for the frequency of CD8+ and CD4+ T cells by flow cytometry. The development of CD4+ FoxP3+ T regulatory subset of cells are further characterized in this system by the induction of red fluorescent protein upon expression of the master regulator FoxP3. This helps with studying the influence of vaccination platform on antigen specific T lymphocytes over time, that have been shown to be critical for effective tumor reduction and control. Additionally, the weight of each animal is tracked at the time of blood draw, as an indicator of overall health throughout the study.

TABLE 1
Study protocol for dose-range finding study in mice

| Purpose | To determine the dose of OX40L-Ig when co-administered with a fixed amount | |
|---|---|---|
| | of gp96-Ig secreting cells. | |
| Materials | Animals | |
| | 48 C57BL/6 mice purchased and acclimated to our facility for 3 | |
| | days (per IACUC) | |
| | 10 OT-I/GFP mice purpose bred in-house | |
| | 10 OT-II/RFP mice purpose bred in-house | |
| | Cells | |
| | mHS-110 (B16F10) cells | |
| | mHS-130 (B16F10) cells | |
| | Consumables | |
| | CD4+ negative selection isolation kit | |
| | CD8+ negative selection isolation kit | |
| | Antibodies | |
| | αCD4-AF647 | |
| | αCD8-BV421 | |
| | αCD3-AF700 | |
| | 28-gauge insulin syringes | |
| | HBSS | |
| | FBS | |
| | EDTA solution | |
| | FACS Buffer | |
| | mytomycinC | |
| | Equipment | |
| | Pipettes | |
| | Centrifuge | |
| | 50 ml conical tubes | |
| | 1.5 ml tubes | |
| | 0.7 micron cell strainers, sterile | |
| Reagent | FACS Buffer | |
| Preparation | In a 500 ml bottle of 1X PBS add BSA powder to 2% w/v. Stir | |
| | gently until fully dissolved. Add sodium azide to .01% v/v and | |
| | EDTA solution to 1 mM. Filter sterilize and store at 4° C. | |
| | Flow Cocktail Master Mix | |
| | A 1X master mix for this panel is made as follows: to 41 μl of | |
| | FACS buffer add 2 μl of aCD4, 2 μl of αCD3, and 3 μl of | |
| | αCD8. Scale up as needed for the number of samples to be | |
| | stained. | |
| Instrument | The Sony SH800 cytometer must pass calibration on each day of acquisition. | |
| Preparation | Data is acquired into a template cytometry experiment previously prepared by a | |
| | skilled operator. The template has the appropriate single color compensation | |
| | controls acquired and compensation matrix parameters calculated and applied. | |
| | The template has a minimum of 30,000 CD3+ gated events as the stopping | |
| | condition for each sample. | |
| Study Groups | Group 1 Ratio of gp96-Ig:OX40L-Ig = 1:0.01 | |
| | Group 2 Ratio of gp96-Ig:OX40L-Ig = 1:0.1 | |
| | Group 3 Ratio of gp96-Ig:OX40L-Ig = 1:1 | |
| | Group 4 Ratio of gp96-Ig:OX40L-Ig = 1:10 | |
| | Group 5 Ratio of gp96-Ig:OX40L-Ig = 1:100 | |
| | Group 6 Control (gp96-Ig alone) | |
| Protocol | 1. | Day −2 |

| | |
|---|---|
| a. | Sacrifice OT-I/GFP mice and remove spleens. |
| b. | Prepare a single cell suspension from pooled spleens and |
| | perform a CD8 enrichment with the appropriate isolation kit. |
| c. | Wash the enriched cell pool in HBSS and resuspend at 4 × 10⁷ |
| | cells/ml in HBSS. This is Pool1. |
| d. | Place on ice. |
| e. | Sacrifice OT-II/RFP mice and remove spleens. |
| f. | Prepare a single cell suspension from pooled spleens and |
| | perform a CD4 enrichment with the appropriate isolation kit. |
| g. | Wash the enriched cell pool in HBSS and resuspend at 4 × 10⁷ |
| | cells/ml in HBSS. This is Pool2. |
| h. | Place on ice. |
| i. | Combine equal volumes of Pool1 and Pool2 to form Pool3. |
| j. | Vortex well to mix. |
| k. | Discard any remaining volume of Pool1 and Pool2. |
| I. | Into the tail vein of the C57BL/6 mice inject 50 μl of Pool3. |
| m. | Allow the mice to rest overnight. |

| | |
|---|---|
| 2. | Day −1 |

| | |
|---|---|
| a. | Prepare a staining master mix for the mice in groups 1-6 using |
| | the antibodies described above. Store at 4° C. until ready to |
| | use. |
| b. | Randomly assign the mice to study groups 1-6, 8 mice per |
| | group. |
| c. | Perform tail bleeds from each mouse and stain with the master |
| | mix, following standard lab protocol for cell surface staining. |
| d. | Acquire on the cytometer. |
| e. | Export the FCS files onto the LabServer. |

| | |
|---|---|
| 3. | Day 0 |

| | |
|---|---|
| a. | Harvest the vaccine cell lines and treat with mitomycinC to |
| | render replication incompetent. |
| b. | Resuspend the gp96-Ig secreting cells (mHS-110 (B16F10) at |
| | 4 × 10⁶ cells/ml in HBSS and place on ice until ready to use. |

| | |
|---|---|
| i. | Each mouse receives 250 μl of gp96-Ig cell |
| | suspension, dosing 10⁶ cells per animal. |

| | |
|---|---|
| c. | Resuspend the OX40L-Ig secreting cells (mHS-130 (B16F10) |
| | at 3.4 × 10⁸ cells/ml and prepare dilutions such that each group |
| | receives the amount of cells per animal indicated below in a |
| | volume of 250 μl: |

| | |
|---|---|
| i. | Group 1 - 3.41 × 10³ cells |
| ii. | Group 2 - 3.41 × 10⁴ cells |
| iii. | Group 3 - 3.41 × 10⁵ cells |
| iv. | Group 4 - 3.41 × 10⁶ cells |
| v. | Group 5 - 3.41 × 10⁷ cells |
| vi. | Group 6 - 250 ul of HBSS |

| | |
|---|---|
| d. | Return mice to their cages after each injection and allow to |
| | rest. |

| | |
|---|---|
| 4. | Days 3, 6, 9, 12, 15 |

| | |
|---|---|
| a. | Prepare a staining master mix for the mice in groups 1-6 using |
| | the antibodies described above. Store at 4° C. until ready to |
| | use. |
| b. | Perform tail bleeds from each mouse and stain with the master |
| | mix, following standard lab protocol for cell surface staining. |
| c. | Acquire on the cytometer. |

| | |
|---|---|
| Data Analysis | All FCS files are imported into FlowJo and analyzed by a skilled operator. The |
| | analyst reports the following cell frequencies for each sample acquired: |
| | CD3+/CD8+ |
| | CD3+/CD8+/GFP+ |
| | CD3+/CD4+ |
| | CD3+/CD4+/RFP+ |
| | Data is plotted as frequency of the indicated cell phenotype over time. |

In-Vitro Assay Comparing Signaling Strength of Murine and Human OX40L-Ig in a Human OX40 Receptor Signaling Assay:

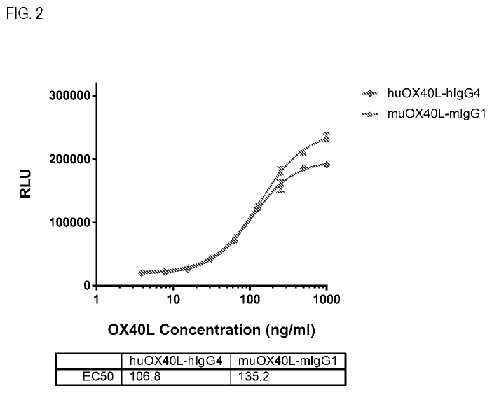

To characterize the signaling activity of OX40L derived from mouse and human species on the human OX40 receptor transfected in the human Jurkat cell line to determine the equivalence of cross-species signaling. A side by side comparison of muOX40L-mlgG1 (mouse derived OX40L), and huOX40L-hIgG4 (human derived OX40L) of NFκB signal induction through human OX40 receptor binding was done using a Jurkat/OX40 cell based system. Cells were stimulated with muOX40L-mlgG1 or huOX40L-hIgG4 with the maximum concentration of 1 μg/ml. Each ligand was tested in duplicate wells on the same plate. The luciferase activation was measured after 5 hours using Bio-Glo reagent from Promega and the relative luminescence was measured using a luminometer. The EC₅₀ values were calculated using a four-parameter logistic curve analysis. The calculated EC₅₀ values indicate that the activity of the mouse and human derived OX40L are very similar against the human OX40 receptor. The EC₅₀ results from this experiment, as shown in FIG. 2, align with the observation of high sequence homology between human and mouse OX40L and suggest that the mouse is a good predictor of clinical pharmacokinetics.

### Example 2: Phase 1 Clinical Trial, Dosage Form, Route of Administration and Dosing Regimen

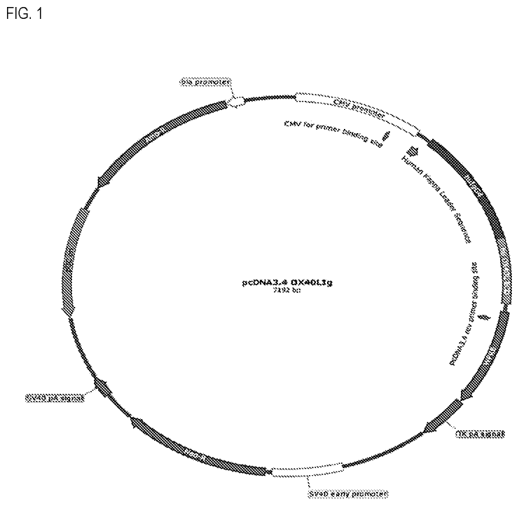

The drug product is a viable whole cell vaccine that has been irradiated to render cell replication-incompetent while expressing the co-stimulatory fusion protein OX40L-Ig, which is the ligand for the OX40 receptor, a member of the TNF-receptor superfamily. The drug product is a viable whole cell vaccine derived from a human lung adenocarcinoma cell line. The cell line is transfected with a 7192 bp plasmid cDNA ‘pcDNA3.4 OX40L-Ig’ stably expressing the cDNA for OX40L-Ig to develop an irradiated whole cell vaccine as shown in FIG. 1. HS-110 refers to (viagenpumatucel-L); genetically engineered human lung adenocarcinoma cell line secreting gp96-Ig fusion protein, and HS-130 refers to genetically engineered human lung adenocarcinoma cell line AD100, secreting OX40L-Ig fusion protein.

A Phase I, first-in-human, dose-escalation study to evaluate the safety and immunologic dose of HS-130 alone and in combination with HS-110 in patients with solid tumors refractory to standard care is undertaken.

Primary Objective: To study safety and tolerability of HS-130 alone and in combination with HS-110 in patients with solid tumors refractory to Standard of Care (SOC).

Secondary Objective: To determine the immunologic dose of HS-130 with a fixed dose of HS-110 administered in combination in patients with solid tumors refractory to SOC. To study the immunological effect generated by HS-130 in combination with HS-110 by determining total peripheral blood mononuclear cell (PBMC) counts and activation state of PBMC subsets using flow cytometry.

Exploratory Objective

1) To evaluate archival tissue for shared antigen expression with HS-110 by RNA-seq; 2) To evaluate immune reactivation response via ELISPOT using IFNγ, granzyme B (gzB) production as functional readouts; 3) To determine the presence of a specific cytokine signature in response to combination treatment with HS-130 and HS-110; and 4) To determine clinical response to combination treatment with HS-130 and HS-110.

Methodology:

This is a first-in-human, Phase I trial of HS-130 alone and in combination with HS-110 in a mixed population of patients with advanced solid tumors refractory to SOC. Both HS-130 and HS-110 are genetically modified, viable, replication-incompetent cancer cells, designed to stimulate an immune response when administered into the intradermal layers of the skin. The purpose of the study is to study the safety and immunological response associated with the treatment. In the study, patients who meet the inclusion/exclusion criteria receive escalating doses of the HS-130 cells using a 3+3 design. The first cohort start treatment with a single fixed dose of HS-130 based on the minimum anticipated biological effect level (MABEL) established in animal models. After a safety assessment interval of 2 weeks following the first dose of HS-130, the patient is administered the same dose of HS-130 along with a fixed dose of HS-110 cells (1×10⁷ cells, which is an established safe dose in the ongoing Phase 2 study of HS-110). In the absence of any safety issues 2 weeks after the combination treatment, the patient can continue the combination treatment of HS-110+HS-130 administered bi-weekly for 6 months or until disease progression, death, patient withdrawal of consent, investigator decision to remove patient, or intolerable toxicity, whichever occurs first.

Three patients are enrolled in each dose cohort. After the first patient, the second and third patients has a 1 week staggered delay following the first patient dose. Once all 3 patients have completed the first cycle of dosing (i.e. 4 weeks, with one dose of HS-130 on Day 1, and one combined dose on Day 15), a review of the safety and tolerability of the treatment administered among the patient cohort is reviewed by an Investigator/Sponsor dose escalation committee. If no Dose Limiting Toxicity (DLT) occurs, then the dose escalation committee may recommend enrollment at the next higher dose level. If DLT occurs in one patient, then up to 3 additional patients is enrolled and treated at the dose level. If less than or equal to one in six patients experiences a DLT, then the dose escalation committee recommends enrollment at the next higher dose level. The conduct and completion of each subsequent dose cohort follows in similar fashion until DLT occurs in 2 patients among a total of up to 6 treated patients at a dose level.

Schematic for treatment regimen for patients in the study:

Should an immunologically active dose be identified before reaching MTD, then the sponsor may decide to discontinue further dose escalation. Any patient who does not complete the first cycle of treatment (i.e. at least one combination dose) is replaced. It is predicted that 4 dose levels of HS-130 are explored, and 12 to 24 patients are enrolled on the trial. All patients are monitored for extensive safety assessment including serum cytokines/chemokines, and immune phenotype profiling of immune cell subsets by flow cytometry. The immune response is evaluated by ELISPOT using HS-110 lysate and HS-110 specific peptides for IFNγ and Granzyme B production. Safety is assessed by frequency of adverse events (AEs), evaluation of clinical laboratory parameters (hematology, and biochemistry), weight, vital signs, electrocardiogram (ECG), performance status, physical exams (PEs), and recording of concurrent illness/therapy and adverse events. CTCAE version 5 is used to grade all toxicities.

Number of Patients: Up to 12-24 total patients are enrolled.

Inclusion Criteria: Patients must meet all of the following inclusion criteria before they are allowed to participate in the trial: Patients with select solid tumor types (defined as those having CTA overexpression overlap with at least 10 CTAs overexpressed by HS-110) who have failed available standard therapy or who are not candidates for standard therapy, and for whom, in the opinion of the investigator, experimental therapy with HS-110+HS-130 may be beneficial. Age ≥18 years. Have an acceptable organ function. Have an Eastern Cooperative Oncology Group (ECOG) performance status of 0 or 1. Life expectancy of at least three months. Have fresh or archival tumor tissue available at screening. Patients, both females and males, of childbearing/reproductive potential must agree to use adequate contraception while included in the trial and for six months after the last treatment with HS-130 and/or HS-110. Patients must sign the informed consent form.

Exclusion Criteria: If any of the following apply, the patient must not enter the trial: Clinically significant cardiac disease, congestive heart failure and/or uncontrolled hypertension. Known or clinically suspected leptomeningeal disease. Stable, previously treated metastases in the brain or spinal cord, are allowed as long as these are considered stable (by CT or MR), and not requiring systemic corticosteroids. History of ≥grade 3 allergic reactions as well as known or suspected allergy or intolerance to any agent given in the course of this trial, live cell therapies, or live vaccines. History of suspected cytokine release syndrome (CRS). Known immunodeficiency disorders. Ongoing or current autoimmune disease (history of checkpoint inhibitor immune related adverse events are permissible if resolved). Any condition requiring concurrent systemic immunosuppressive therapy. Major surgery within four weeks before first IMP administration. Any ongoing anticancer therapy including; small molecules, immunotherapy, chemotherapy, monoclonal antibodies or any other experimental drug. Prior therapy must be stopped within four weeks before first infusion in the study, or 5 half-lives, or twice the duration of the biological effect of the investigational product (whichever is shortest). Adjuvant anti-hormonal treatment(s) for prior breast cancer or prostate cancer are allowed. Known current malignancy other than inclusion diagnosis. Prior curable cancer with complete remission for >2 years is allowed. Any other ongoing significant, uncontrolled medical condition. Received a live vaccine within 30 days prior to first dose of study drug. Clinically significant active viral, bacterial or fungal infection requiring: Intravenous treatment with antimicrobial therapy completed less than two weeks prior to first dose, or Oral treatment with antimicrobial therapy completed less than one week prior to first dose. Prophylactic treatment (e.g., for dental extractions) is allowed. Known positive serology for human immunodeficiency virus (HIV), hepatitis B, or hepatitis C (except in cases of immunity after cured infection). Substance abuse, medical, psychological or social conditions that may interfere with the patient's participation in the trial or evaluation of the trial result. Women who are pregnant or breast feeding. Dose and Mode of Administration: Each patient is administered an intradermal injection of HS-130 on Day 1 and another intradermal injection of HS-130 at the same dose with a fixed dose of HS-110 (1×10⁷ cells) on Day 15. Patients not progressing may continue treatment at the discretion of the investigator.

Dose Limiting Toxicity: DLT is defined as any non-acceptable (as defined below) treatment related toxicity (i.e., not attributable to the active disease, disease-related processes under investigation or intercurrent illness) observed during the first 28 days of study treatment. Ongoing safety events beyond Cycle 1 is reviewed across all cohorts during the study to help inform dose escalation decisions.

DLT includes: Hematological toxicities ≥CTCAEv5 grade 3. Non-hematological toxicity ≥CTCAEv5 grade 3. Any other toxicity (greater than at baseline), considered clinically significant and/or unacceptable, and that does not respond to supportive care and results in a disruption of the dosing schedule of more than 14 days.

DLT excludes: Grade 3 self-limited or medically controllable toxicities (e.g., fever without ≥Grade 3 neutropenia, nausea, vomiting, diarrhea, fatigue). Electrolyte disturbances that are managed to grade 1 or less with supplemental therapy.

Data Monitoring Committee: A Data Monitoring Committee (DMC) evaluates the data obtained at each dose level and recommends whether the dose should be escalated as per protocol, revised to a lower level or intermediate level, halted altogether or more patients are required at the same dose level to evaluate safety. Following each DMC meeting, a sponsor safety committee meeting is held to discuss and confirm actions recommended by the DMC.

Duration of Treatment: Upon completion of the first treatment cycle (i.e. 4 weeks, with one dose of HS-130 on Day 1, and one combined dose with HS-110 on Day 15), in the absence of disease progression or unacceptable toxicity, patients may continue to be treated with the combination of HS-130+HS-110 at the same dose bi-weekly for 6-months or until disease progression, death, patient withdrawal of consent, investigator decision to remove patient, or intolerable toxicity, whichever occurs first.

Criteria for Evaluation:

### Primary Endpoints

Safety and Tolerability: Measured by the frequency of treatment emergent adverse events (TEAEs)/serious adverse events (SAEs), including clinically significant (CS) abnormal laboratory parameters, ECGs, PEs, and vital signs in patients receiving at least 1 dose of study drug.
Maximum Tolerated Dose (MTD; highest dose level at which less than one-third of at least 6 patients experienced a DLT during the first treatment cycle), OR definition of an immunologically active dose that makes further dose escalation redundant (i.e. plateau of immune markers or trigger of immunosuppression).

### Secondary Endpoints

Peripheral blood IR analysis of surface markers, CD3, CD4, CD8, CD19, CD25, CD39, CD45, CD56, CD73, FoxP3, Ki-67, and ICAM-1 as surrogates for T-cell activation and proliferation.

### Exploratory Endpoints

Bioinformatic analysis of RNA-seq data generated with comparison to historical gene expression data.
Calculation of responding cell numbers over the course of treatment after subtraction of appropriate patient controls.
Analysis by Luminex multiplex panel to detect changes in pro-inflammatory serum cytokines before and after treatment with HS130 and HS110.
Patient disease status is monitored by clinical and radiological assessment as per the institutional standard. Patient clinical response is evaluated as complete response, partial response, stable disease, or progressive disease per Investigator assessment.

Safety: All patients who have received any component of study treatment are considered evaluable for safety. Safety is assessed by means of physical examination, weight, vital signs, performance status, laboratory evaluations (hematology, biochemistry including cytokines and C-reactive protein), electrocardiogram (ECG), and recording of concurrent illness/therapy and adverse events. CTCAE version 5 are used to grade all toxicities. All related adverse events are monitored until resolution. Patients are monitored for safety and concomitant medications throughout the study. Cytokines (e.g., IFNγ, IL-1β, IL-2, IL-4, IL-6, IL-10, IL-12 p70, IL-17A, TNFα) are be monitored once after each dose and repeatedly in relation to clinically observed reactions after drug injections.

Statistical Methods: All analyses are descriptive. Categorical variables are presented with numbers and, if meaningful, percentages. Continuous variables are presented by n, mean, median, standard deviation and range (min and max) as appropriate. Presentations are by each dose cohort. Efficacy: This is an exploratory trial and therefore no sample size calculations have been performed. Safety Population: All patients who receive at least 1 dose of study drug are evaluated for safety.

### Example 3: Mouse HS-110 (B16F10-OVA-Gp96) and Mouse HS-130 (B16F10-OVA-OX40L) Immunization at a “Narrow” Dose-Range to Correlate CD8+ T-Cell Expansion to Tumor Growth

The experiments of this example demonstrate a best ratio of a mouse HS-110 (mHS-110; B16F10-OVA-gp96-Ig) to a mouse HS-130 (mHS-130; B16F10-OVA-OX40L) for the generation of a primary and secondary tumor-specific CD8+ T-cell response, and demonstrate how anti-tumor T-cell expansion correlates to tumor growth control, in vivo.

As disclosed in the prior examples, HS-110 is a lung adenocarcinoma cancer cell line. HS-110 secretes gp96-Ig, which presents antigens to prime and expand CD8+ T cell responses. Preclinical studies have suggested that the addition of secreted T cell costimulatory molecules, OX40L-Ig in combination with Gp96-Ig in a cellular system expressing a defined antigen, enhances T cell immunity and results in elimination of tumors (Fromm G et al., Cancer Immunol Res. 2016 Sep. 2; 4(9):766-78.).

Therefore, a mouse cell line was devised that secretes only OX40L-Ig, herein referred to as “mouse HS-130” or “mHS-130”, which was used in combination with mHS-110 (See Example 1, above). The best ratio of mHS-110 to mHS-130 for the generation of a primary and secondary CD8+ or CD4+ T cell pool was investigated in the following experiments. The experiments of this example further included a tumor challenge arm to better understand how in vivo expansion of CD8+ T cells correlates to anti-tumor immune responses and tumor growth control.

Experimental Design and Methodology

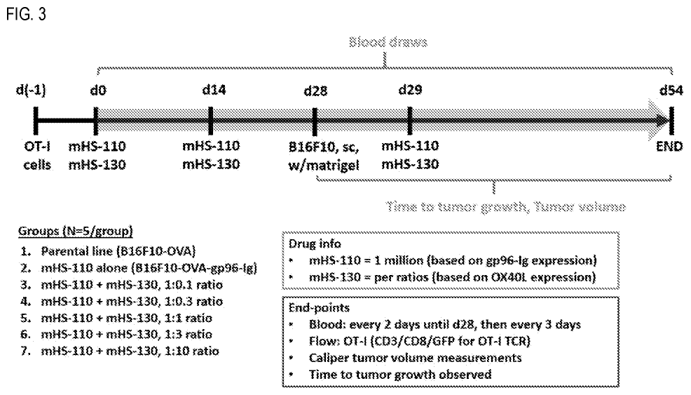

An image showing the full study design can be found in FIG. 3.

OT-1 purification, adoptive T-cell transfer, mHS-110/mHS-130 dosing, and flow cytometry staining: T-cell receptor (TCR) transgenic mouse CD8+(OT-I) cells were isolated from OT-I-GFP bred mice using Easy Sep Mouse CD8 T Cell isolation kit (cat #19853A) and injected into each C57BL/6 mouse intravenously (i.v) through lateral tail vein with 1 million OT-I cells suspended in HBSS (GIBCO 14175-095). Two days after injecting OT-I, all the mice were tail bled for baseline and 4 hours later, mHS-110 (B16F10-OVA-gp96-Ig) cells and mHS-130 (B16F10-OVA-OX40L-Ig) were treated with 10 μg/mL of Mitomycin-C(Sigma-Aldrich cat #M0503) for 3 hours and given intraperitoneally (i.p.) to each group accordingly. Mice were divided into 7 groups with 5 mice per group, except for the parental group which had 3 mice. Animals were dosed based on nanogram expression level (per 10⁶ cells per 24 hr) of gp96-Ig or OX40L-Ig per the design shown in FIG. 3 and Table 2 below.

TABLE 2
Animal Dosing Design

| | | | | | | Ratio | |
|---|---|---|---|---|---|---|---|
| | mHS110 | mHS130 | Parental | GP96 | OX40 | mHS110 to | |
| Group | Cells | Cells | B16 F10-OVA | ng | ng | mHS130 (ng) | Animals |
| A | 1.00E+06 | 3.41E+04 | 0 | 290 | 29 | 1:0.1 | 5 |
| B | 1.00E+06 | 1.02E+05 | 0 | 290 | 87 | 1:0.3 | 5 |
| C | 1.00E+06 | 3.41E+05 | 0 | 290 | 290 | 1:1 | 5 |
| D | 1.00E+06 | 1.02E+06 | 0 | 290 | 870 | 1:3 | 5 |
| E | 1.00E+06 | 3.41E+06 | 0 | 290 | 2900 | 1:10 | 5 |
| F | 1.00E+06 | 0 | 0 | 290 | 0 | — | 5 |
| G | 0 | 0 | 1.00E+06 | 0 | 0 | — | 3 |

Mice were tail bled consecutively on days 3, 5, 7, 10, and 14 post-immunization, and days 17, 19, 21, 24, 28, 33, 38 and 41 post-boosts into heparinized PBS (10 units/ml), and lysed using ACK lysis buffer (150 mM NH₄Cl, 100 mM KHCO₃ and 10 mM EDTA 0.2 Na, pH 7.2) for 3 minutes, and neutralized with 1×PBS. Samples from the OT-1 transfer experiments were then centrifuged at 300 g for 5 minutes, supernatant was removed, and the cell pellet stained with anti-CD3 (20 μg/mL), anti-CD4 (40 μg/mL) and anti-CD8 (5 μg/mL) antibody cocktail made in FACS buffer using Alexa Fluor 700 anti-mouse CD3 (Bio legend cat #100216), Alexa Fluor 647 anti-mouse CD4 antibody (Biolegend Cat #100530) and Brilliant Violet 421 anti-mouse CD8alpha antibody (Biolegend Cat #100738) for 30 minutes at 4° C.

Intracellular Cytokine Staining and Flow Cytometry: Splenocytes (1×10⁶) were incubated with synthetic peptides; SIINFEKL (SEQ ID NO:41), gp100, TRP-1, TRP-1-Variant, or TRP-2 in the wells of a 96-well plate at 37° C. and 5% CO2. Synthetic peptides were added to a final concentration of 0.5 μM with Golgi stop and incubated for 4-10 hours depending on the peptide. Plates were spun, medium was removed, and cells were resuspended with surface markers CD8 and CD3 and incubated at 4° C. for 20 minutes. Cells were washed, resuspended in 50 μl of BD Cytofix/Cytoperm, and incubated at 4° C. for 20 min before another two washes and staining with anti-IFN-γ. Cells were washed once before acquisition and analysis of fluorescence. Analysis was done using FlowJo software (Tree Star Inc.); events were gated for live lymphocytes on FSC×SSC followed by CD8+ cells using CD8×CD3 and displayed as CD8×IFN-γ.

B16F10-OVA tumor challenge and volume calculations: Melanoma B16F10 cells were harvested and resuspended at a concentration of 5×10⁵ cells/100 μl in a volume of 80 μl HBSS and 20 μl Matrigel. C57BL/6 mice were subcutaneously injected with 100 μl of B16F10 cells (5×10⁵ cells/mouse) on the inner abdomen 29 days post-OT-1 transfer and 28 days post-primary vaccination, designated “day 28”, as shown in study design (FIG. 3). The tumor size was measured and documented every 3 days with a caliper, starting on day 7, and calculated using the formula (A×B; A as the largest and B as the smallest diameter of tumor). Tumor growth was documented as standard error mean. To record the survival of the tumor-bearing mice, either natural death or a tumor volume greater than 450 mm² leading to death was counted as death. Each experimental group included five animals.

Tumor tissue digestion for Tumor Infiltrating Lymphocytes (TILs): The MACS Miltenyl Biotec tumor dissociation kit was used for this procedure (cat #130096-730).

ELISPOT Assay: Splenocytes were harvested and red blood cell lysis buffer (cat #36858500, Roche) was used to eliminate red blood cells. Cells were washed in IMDM medium and pelleted. Counted cells were resuspended in IMDM with 10% FBS. Each ELISPOT well received 1 million cells, in a total volume of 200 μl. Treatments included the use of B16F10-OVA parental line lysates (vial of 10 million cells; freeze-thawed three times) at a 10-fold dilution, immunodominant epitopes for B16F10 tumors: “gp100/pmel” (EGSRNQDWL (SEQ ID NO:38)), “TRP-1/gp75” (TWHRYHLL (SEQ ID NO:39)), TRP-2 (SVYDFFVWL (SEQ ID NO:40)) and the MHC-I restricted peptide for H2^{b} haplotype for OVA (SIINFEKL (SEQ ID NO:41)). All peptides were used at a final concentration of 10 μg/mL. PMA/lonomycin was used as a positive control at “1 to 400 dilution” 100 nM of PMA, 1.6 μM ionomycin (500× stock from eBioscience, cat #00-4970-03, and whole chicken ovalbumin protein “OVA” at 250 μg/ml.

Interferon-gamma capture ELISPOT assay was carried out as previously published (Klinman D M et al., Current Protocols in Immunology. 1994 June (1): 6.19.1-6.19.8). Briefly, MAIP N45 Milipore 96-well filtration plates were coated with anti-mouse IFN-γ purified monoclonal antibody (clone AN18) in PBS saline at 10 μg/mL, 50 μl/well, at 4° C., overnight. The next day, cells were washed in IMEM medium plus 10% FBS at 200 ul/well, washed three times, then blocked for 1 hour at 25° C. Cells were processed as described above and 1 million or 100,000 splenocytes were added to each well and treated per the description above with various peptide stimulations. Each plate was incubated in a 37° C., 5% C02, non-vibrating incubator for 16 hrs or overnight. At the end of the incubation, cells were flicked into the sink to stop the culture and washed with PBS+0.1% Tween-20 three times and blotted hard onto paper towels to get rid of residual liquid after the last wash. Secondary biotinylated antibody was added at 50 μl per well, 4 μg/mL of anti-IFN-γ (clone AN18), incubated at 25° C. for 2 hrs. Plates were then washed again with PBS+0.1% Tween-20 three times. Peroxidase-conjugated streptavidin from Jackson research labs was then added at a 1 to 1000 dilution from stock concentration in PBS+0.1% Tween-20+2% BSA, 50 μl per well, 30-minute incubation at 4° C. Plates were then washed with PBS+0.1% Tween three times. The plastic backing of the ELISPOT plate was removed and the entire plate was submerged and soaked in PBS+0.1% Tween-20 for 1 hour at 25° C. The plate was then washed in PBS to remove the tween, then 100 μl of the AEC substrate (Vector kit) added in 100 mM of TRIS buffer at pH 8.2 was added to each well to allow for development of spots. At the end of the incubation, 10-20 minutes, all plates incubated for the same time, the reaction as stopped by rinsing the plates with tap water and air dried.

Plates were scanned and counted in an AID Autoimmun Diagnostika GMBH iSpot monochromatic ELISPOT reader (2018). Counting parameters for each plate remained consistent across all plates that were developed together as to prevent bias, as spot development, density and magnitude is influence by the operator, reagent lots and final substrate development time.

### Experimental Results

Naïve OT-I GFP+CD8+ T cells expressing Vα2/Vβ5 and specific for H-2K^{b}/OVA₂₅₇₋₂₆₄ were adoptively transferred into naïve WT C57BL/6 mice. Recipient mice were injected with mHS-110 at a fixed dose of 1 million cells (290 ng of gp96-Ig) with different ratios of mHS-130, in which a 1 to 1 ratio of mHS-110 to mHS-130 was equivalent to 290 ng of gp96-Ig to 290 ng of OX40L.

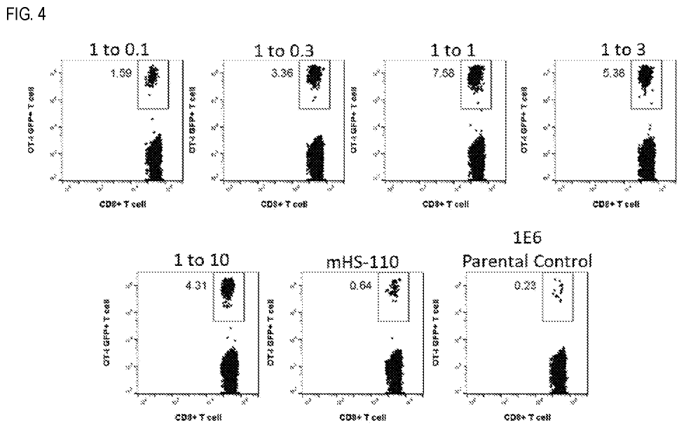

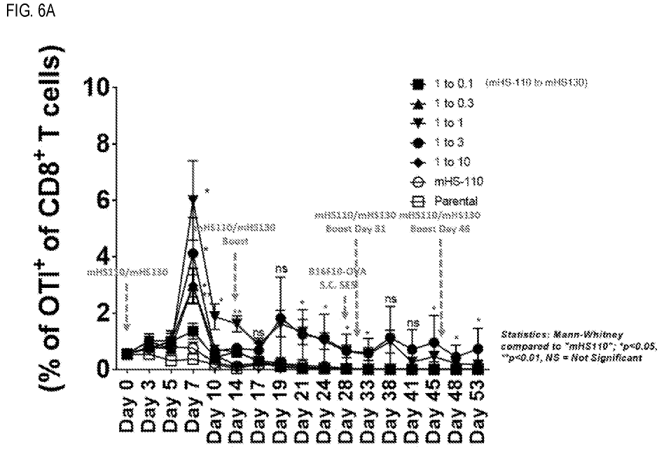

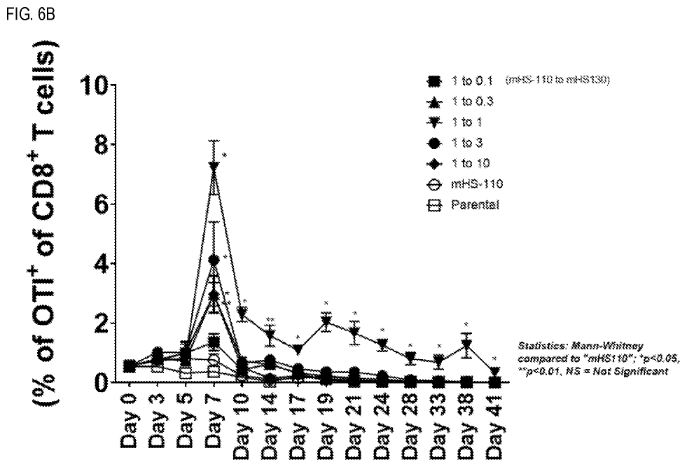

After vaccination, OT-I GFP+CD8+ T cells were analyzed in the blood on days 0, 3, 5, 7, 10, and 14, representing the acute expansion phase and the contraction phase of CD8+ T cell responses. Based on accumulation of the transferred cells, the generation of the primary effector pool was found to peak on day 7 (FIG. 4, FIG. 6A, FIG. 6B) and contract by day 14 post-vaccination (FIG. 6A, FIG. 6B). Significant increases were found on day 7 in the 1 to 1, the 1 to 3 and the 1 to 10 ratios of mHS-110 to mHS-130 (FIG. 6A, FIG. 6B) (*p<0.05; **p<0.01). Furthermore, the 1 to 1 ratio had significant increases on days 10 and 14 post-vaccination (*p<0.05).

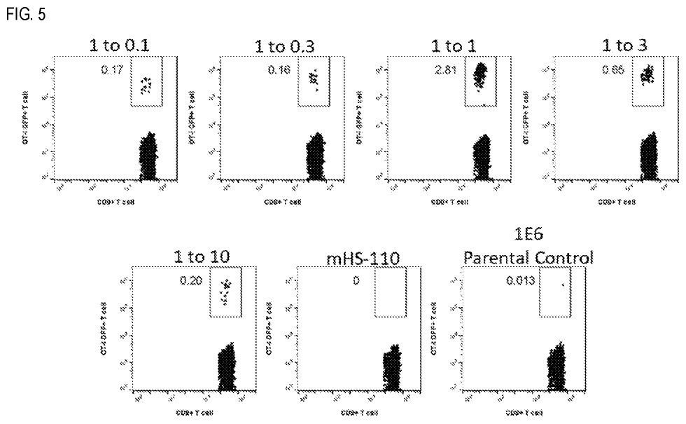

Following this analysis, a boost vaccination was given on day 14, and OT-I GFP+CD8+ T cells were analyzed in the blood on days 17, 19, 21, 24, and 28, representing the secondary expansion phase and the contraction phase of CD8+ T cell responses (FIG. 5, FIG. 6A, FIG. 6B). Based on accumulation of the transferred cells, the generation of the memory effector pool was found to peak and increase in percentages by day 21 post-challenge and contract by day 28 post-challenge. Significant increases were found on day 21, 24 and 28 in the 1 to 1 ratio of mHS-110 to mHS-130 (FIG. 6A, FIG. 6B) (*p<0.05).

With each subsequent boost of mHS-110 with mHS-130, the expansion of antigen-specific T-cells became less. Tumor was given s.c., at 500,000 B16F10-OVA cells per mouse on day 28, with another boost of mHS-110 with mHS-130 given on day 31. This resulted in a non-significant increase in antigen-specific CD8+ T-cells (FIG. 6A, FIG. 6B), however without outliers removed, the 1 to 1 ratio provided the best and most consistent response to vaccine re-challenge (FIG. 6B).

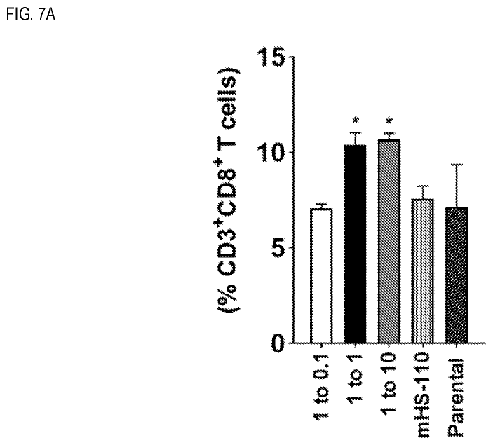

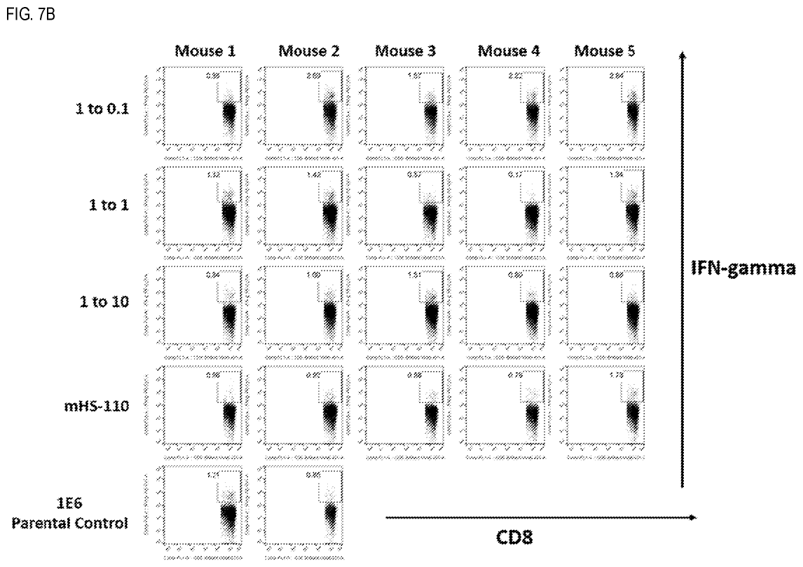

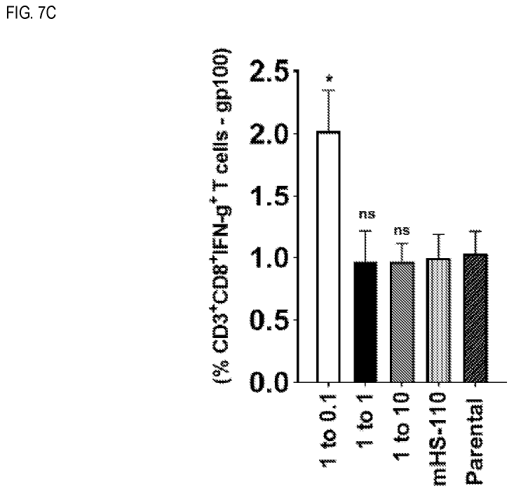

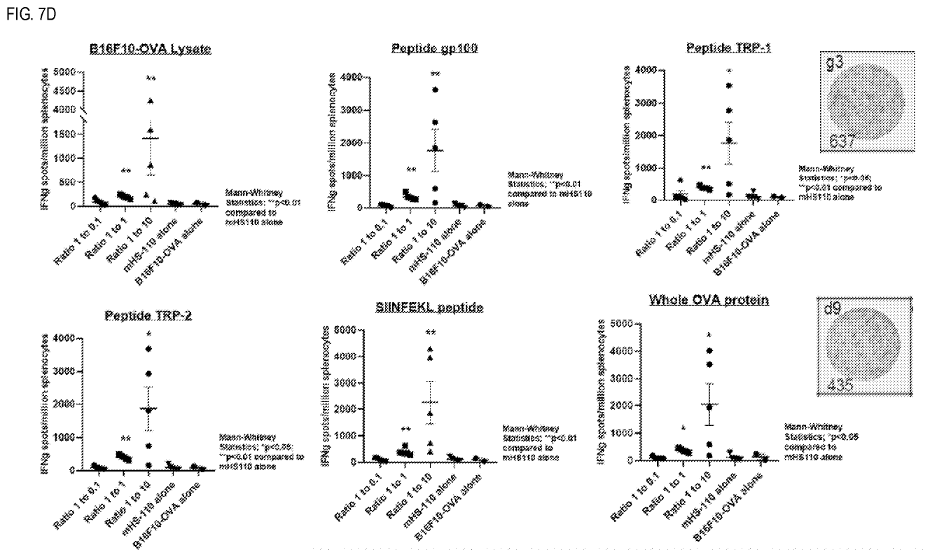

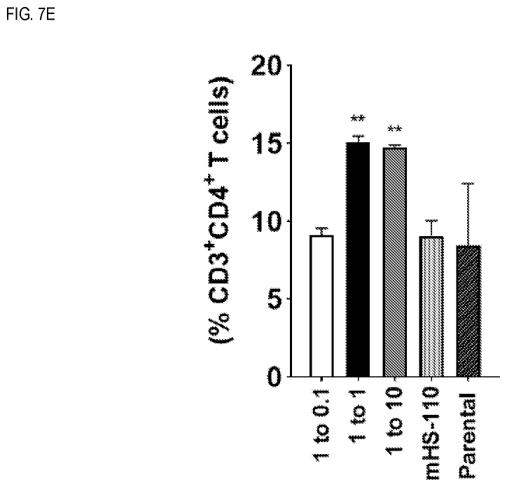

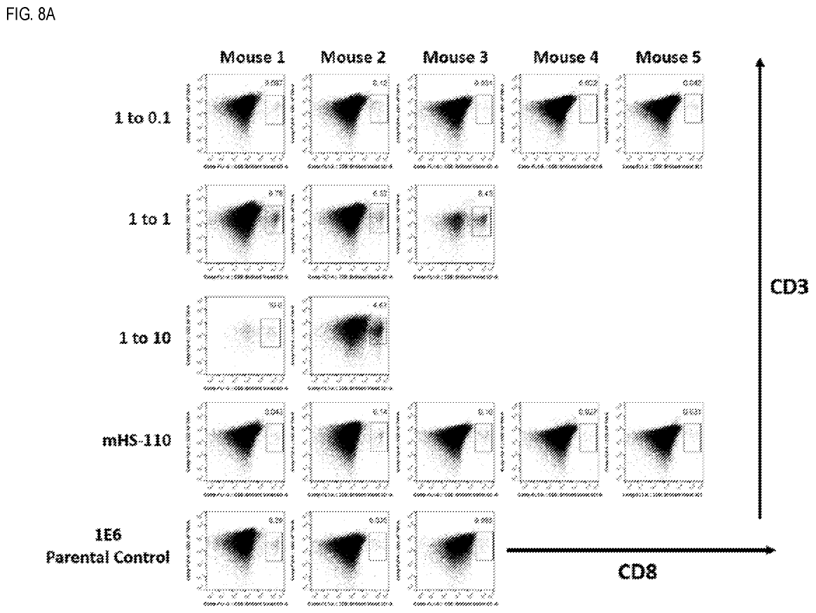

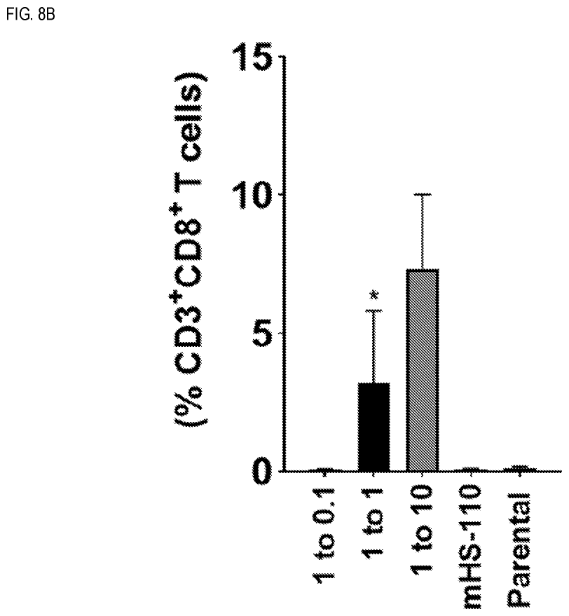

Looking at the endogenous response, at day 54, the end-of-study, the percent of CD8+ T-cells is elevated in only those ratio groups that showed the greatest anti-tumor response, 1 to 1 and 1 to 10 (FIG. 7A), *p<⁰0.05 as compared to mHS-110 alone. Splenocytes rechallenged, with B16F10, H2K^{b}restricted, immunodominant peptide, gp100, directly ex vivo, generated IFN-γ release by tumor-specific CD8+ T-cells, however, all groups except the 1 to 0.1 group showed no difference compared to mHS-110 alone, suggesting that such antigen specific T-cells most likely migrated directly to the tumor, which resulted in the observed growth retardation (FIG. 7B, FIG. 7C). This is supported by data showing an increase in CD8+ TILs in remaining tumors (FIG. 8A). Other peptides were also tested including TRP-2, TRP-1, and TRP-1-variant. The H2K^{b} restricted peptide of OVA, SIINFEKL (SEQ ID NO:41), was also tested, however, OT-1 frequencies were very low at the day 54 timepoint, that only a weak response was noted (data not shown).

Endogenous immune responses to vaccination and tumor burden was examined using end-of-study ELISPOTs from splenocytes. In agreement with the reported flow cytometry results and tumor growth, responses to tumor lysate, various B16F10 immunodominant peptides and OVA all followed the predicted ratios and appeared to be dependent on the presence of mHS-130 (FIG. 7D). Responses to various stimulants were significant compared to mHS-110 alone by the Mann-Whitney non-parametric statistical test.

With regard to flow cytometry plots, endogenous CD4+ T-cells expanded in frequency for the 1 to 1 and 1 to 10 ratio dose groups, suggesting the need for Th1 help to generate anti-tumor cytotoxic T-lymphocytes, in vivo (FIG. 7E). Effector (CD62L^{lo} CD44^{hi}) and naïve (CD62L^{hi} CD44^{lo}) CD4+ and CD8+ endogenous T-cell frequencies were also measured; and no biologically significant response was found between the groups (data not shown). Also, no biologically significant response was seen for PD-1+CD8+ endogenous T-cells nor ILRG+/−IL-7R+/− memory CD8+ T-cell subsets, for any group tested (data not shown).

Tumor infiltrating lymphocytes (TILs) were quantitated for each remaining tumor mass, per animal, at the end of the study, day 54. The proportion of CD8+ TILs in the 1 to 1 and 1 to 10 groups were 51-fold and 118-fold, respectively, over that of the 1 to 0.1 vaccination ratio of mHS-110 to mHS-130 (FIG. 8A, FIG. 8B). The 1 to 1 group had two animals that failed to establish stable tumors (full tumor growth inhibition), and the 1 to 10 group had three animals with full tumor growth inhibition. For this reason, proper statistics for TIL percentages cannot be performed with less than three samples, thus the 1 to 10 group is ineligible for statistical analysis. However, comparing the 1 to 1 group to mHS-110 alone (FIG. 8A, FIG. 8B; *p<0.05), it was observed that significantly more CD8+ TILs were found in these tumors, and this was true when compared to the 1 to 0.1 group as well. In both cases, there was a dose-dependent increase in the proportion of CD8+ TILs with vaccination ratio with the 1 to 1 and 1 to 10 ratio giving the best response.

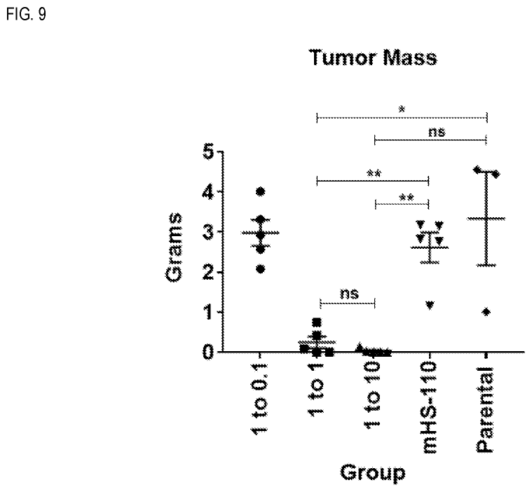

Tumor volume was measured over time, from the point of tumor cell inoculation until end of study, a total of 25 days of logarithmic growth. Since this was a protection, prophylaxis, study, tumor delay was measured, rather a therapeutic response to established tumors. As shown in FIG. 10A, only two ratios, 1 to 1 (290 ng of gp96-Ig to 290 ng of OX40L-Ig) and 1 to 10 (290 ng of gp96-Ig to 2900 ng of OX40L-Ig) of mHS-110 to mHS-130, gave a consistent and significant tumor growth inhibition as compared to the mHS-110 group alone, with greatest separation observed for days 21-25 (**p<0.01). Individual tumor growth curves are shown in FIG. 10B to show individual animal variance. Measuring end-of-study tumor mass confirmed what tumor volume had demonstrated, in that both the 1 to 1 and 1 to 10 ratio gave the best response, as compared to mHS-110 alone (FIG. 9). For tumor volume, the 1 to 1, and 1 to 10 ratio were not significantly different.

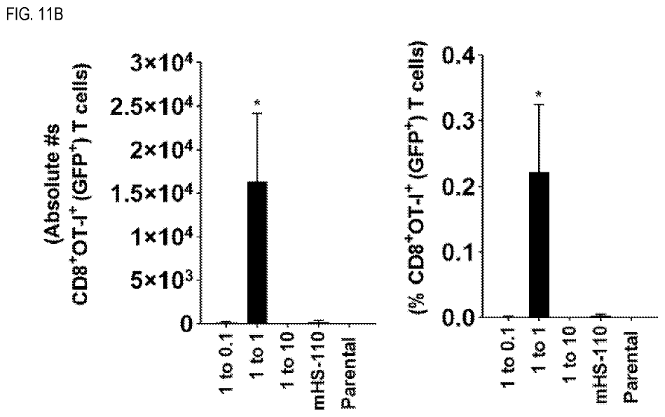

Tumor growth inhibition, ex vivo anti-tumor T-cell responses and TIL infiltration correlated with reduced PD-1+expression on CD8+ T-cells, observed in the 1 to 1 dose ratio group (FIG. 12A, FIG. 12B; *p<0.05). Although not significantly different from mHS-110 alone group, the 1 to 10 ratio also showed a trend decrease in PD-1 expression. Expression of PD-1 occurs during initial T-cell activation, but under constant re-stimulation or in the presence of cognate antigen, PD-1 is expressed by exhausted T-cells (Simon S. et al., Oncoimmunology. 2018 Sep. 7 (1): e1364828). Elevated expression of PD-1 was seen in those groups with the greatest tumor burden (1 to 0.1, mHS-110 alone and parental B16F10-OVA alone), which suggested that cells have become exhausted from continued anti-tumor fighting, but those that have received proper stimulation with OX40L, via mHS-130 vaccination, show lower PD-1 expression and less tumor burden. This may explain why the 1 to 1 and 1 to 10 ratio dose groups showed the lowest PD-1 expression in the spleen. Expression of PD-1 was also measured in the tumor, by looking at TILs, but the frequency was below the limit of quantitation making analysis difficult (data not shown).

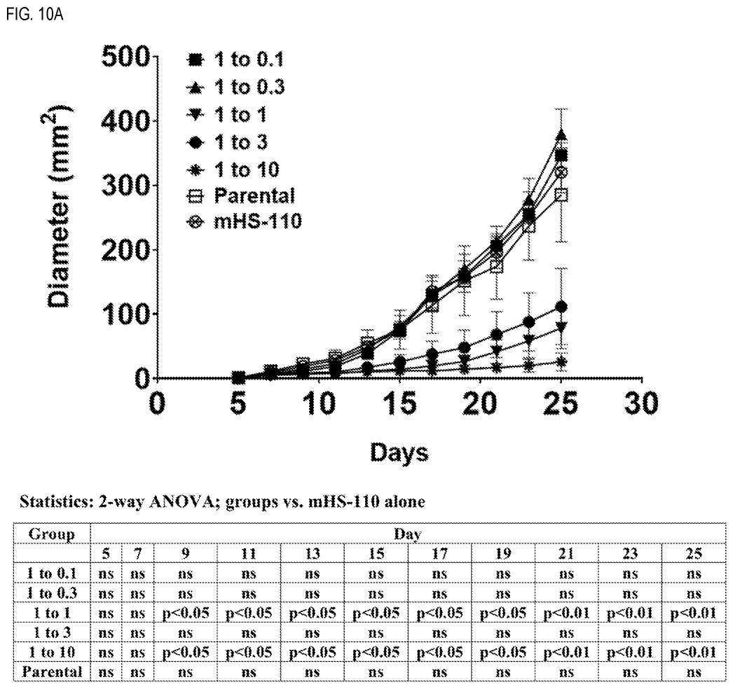

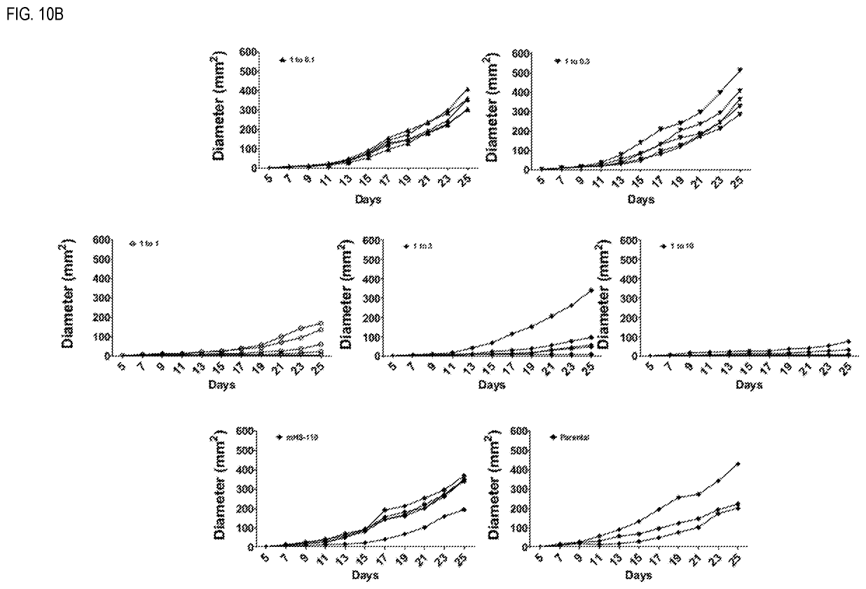

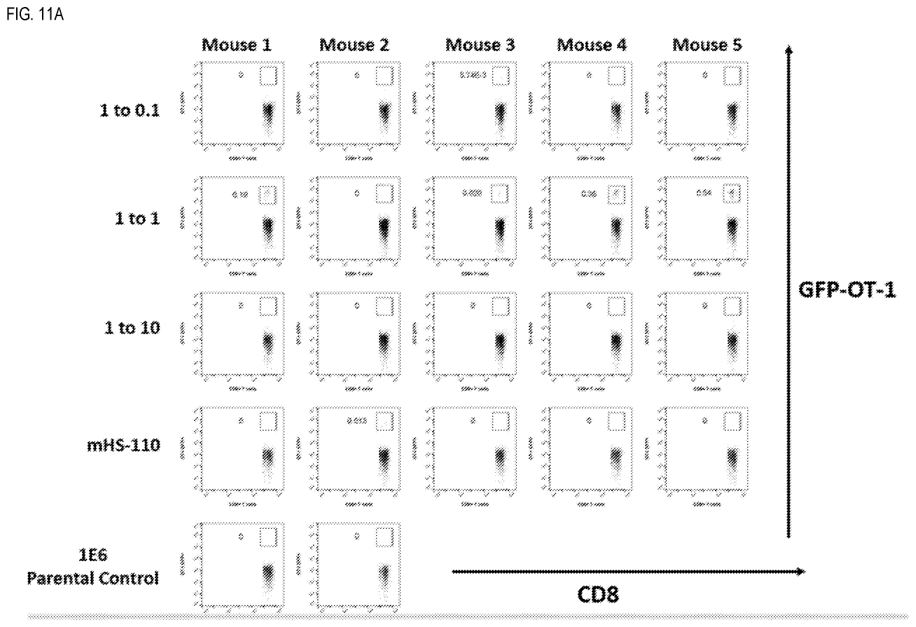

Day 54 spleen OT-1 frequencies and absolute counts were measured for remaining groups at the end of the study. Only the 1 to 1 ratio produced long-lived circulating OT-1 cells above all other groups tested (FIG. 11A, FIG. 11B; *p<0.05). This data strongly suggested that the 1 to 1 ratio of gp96-Ig to OX40L-Ig produces the best anti-tumor T-cell expansion response for both immediate and long-term immune responses, and that this directly correlates to tumor growth inhibition, in vivo.

### Example 4: Study of Gp96-Ig (mHS-110) to OX40L-Ig (mHS-130) Dose Ratios

In this example, experiments were conducted to determine best gp96-Ig (mHS-110) to OX40L-Ig (mHS-130) dose ratios that result in CD8+ T cell expansion and tumor growth inhibition. HS-110 is a lung adenocarcinoma cancer cell line that secretes gp96-Ig, which presents antigens to prime and expand CD8+ T cell responses. In the present example, a mouse cell line (mHS-130) was developed that secretes only OX40L-Ig.

In the present study, mHS-130 (secreting gp96-Ig fusion protein) was used in combination with mHS-110 (secreting OX40L-Ig fusion protein). An objective of the study was to determine ratio of mHS-110 to mHS-130 most suitable for generation of a primary and secondary CD8+ or CD4+ T cell pool. Thus, this study tested in tumor-bearing animals a variable ratio, in a dose-escalation manner, to determine the ratio(s) and dose(s) that result in the most effective CD8+ T-cell expansion and tumor growth inhibition combination.

### Experimental Design and Methodology

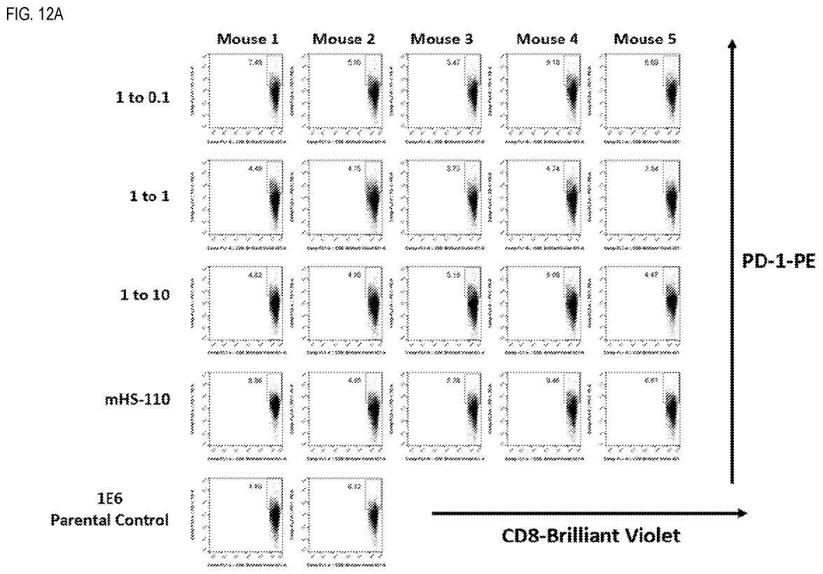

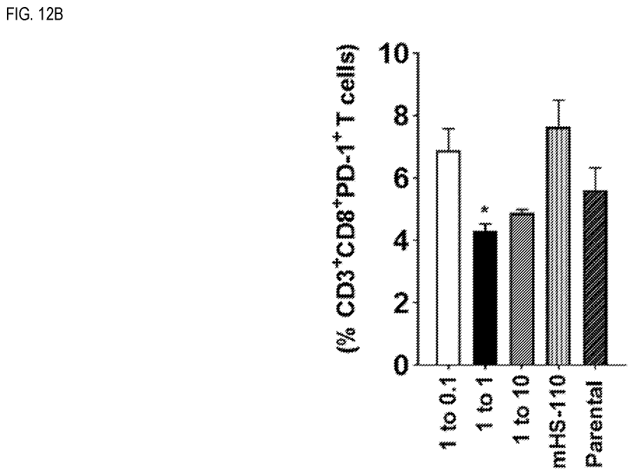

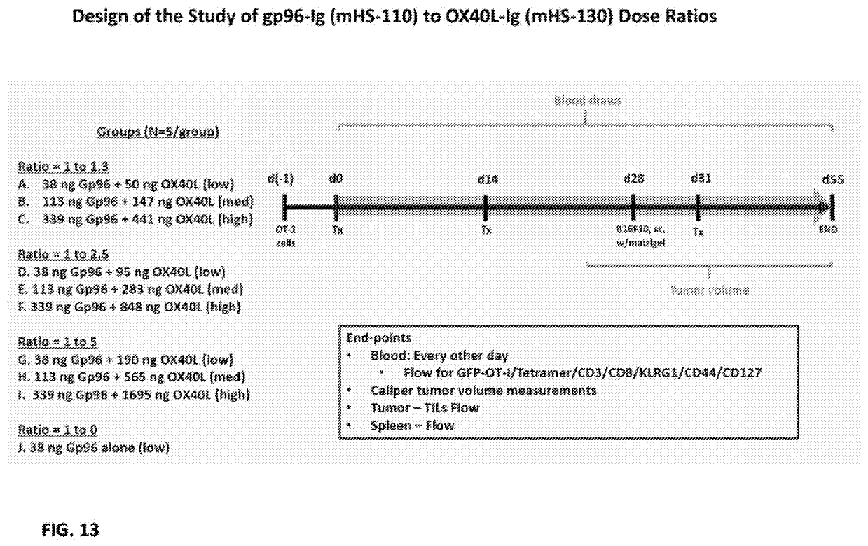

FIG. 13 illustrates a design of the present study.

OT-1 Purification, Adoptive T-Cell Transfer, mHS-110/mHS-130 Dosing, and Flow Cytometry Staining

T-cell receptor (TCR) transgenic mouse CD8+(OT-I) cells were isolated from in-house bred OT-I-GFP mice using Easy Sep Mouse CD8+ T Cell isolation kit (cat #19853A) and injected into each C57BL/6 mouse intravenously (i.v.) through lateral tail vein with 1 million OT-I cells suspended in HBSS (GIBCO 14175-095). Two days after injecting OT-I, all the mice were tail bled for baseline and 4 hours later, mHS-110 (B16F10-OVA-gp96-Ig) cells and mHS-130 (B16F10-OVA-OX40L-Ig) were treated with 10 μg/mL of Mitomycin-C(Sigma-Aldrich cat #M0503) for 3 hours and given intraperitoneally (i.p.) to each group accordingly. Mice were divided into 10 groups with 5 mice per group. Three different treatment ratios were provided with escalating doses within each ratio group and all compared against mHS-110 (gp96-Ig) alone, the control group (ratio 1 to 0). Animals were dosed based on nanogram expression level (measured as ng/10⁶ cells/24 hrs) of gp96-Ig or OX40L-Ig, as shown in Tables 3 and 4 below. As shown in the study design FIG. 13, dose ratios of gp96-Ig to OX40L-Ig were 1:1.3, 1:2.5, 1:5, and 1:0 (mHS-110 (gp96-Ig) alone), and each dose was tested at three different dose levels (“low,” “medium,” and “high”). Mice were boosted on days 14 and 31 with the same ratios of mHS-110 and mHS-130 as in the primary phase, and OT-I GFP+CD8+ T cells were analyzed in the blood days post-challenge. Tumor was provided on day 28. Consecutive bleeds were collected from the peripheral blood and analysis was performed by flow cytometry on both exogenous, adoptively transferred, OT-1 and endogenous CD8+ and CD4+ T-cells for activation, and short (SLECs) and long-term (MPECs) memory markers as outlined in the methods section. Tumor growth kinetics, response rates and infiltrating lymphocytes, were also quantitated.

Cell Line Protein Expression Data for mHS-110 and mHS-130

The amount of murine gp96 protein expressed by the mHS-110 cells was determined by ELISA. For each sample to be tested, one million B16F10-Ova9 parental and mHS-110 cells were plated in a 6-well tissue culture plate in a total volume of 1 ml each. Cells were incubated at 37° C. with 5% CO₂ for 24 hours at which point the supernatants were harvested. Supernatants were then centrifuged at 2500 rpm for 5 minutes to pellet any cell debris. Clarified supernatants were then transferred to new 1.5 ml tubes and stored at −80° C. Each sample tested was from a fresh vial of mHS-110 cells thawed and expanded.

To perform the ELISA, 96-well plates (Corning, cat #9018) were coated with 2 μg/ml of sheep anti-gp96 (R&D Systems, cat #AF7606) in carbonate-bicarbonate buffer. Plates were sealed and stored at 4° C. overnight. Plates were then washed 4 times with 1×TBST (VWR, cat #K873) and then blocked with 1× casein solution (Sigma-Aldrich, cat #B6429) for 1 hour at room temperature. The plates were then washed 4 times with 1×TBST and a human gp96-mouse Fc standard (Thermo Fisher Scientific, lot #2065447) was prepared in IMDM (Gibco, cat #12440-053) with 10% FBS (Gibco, cat #10082-147). A 2000 ng/ml human-gp96-mFc standard solution was made and 2-fold serial dilutions were performed down to 1.95 ng/ml. Sample supernatants were loaded onto the ELISA plates starting at a 1:2 dilution and then 2-fold serial dilutions were performed to a highest dilution of 1:16. Plates were sealed and incubated for 1 hour at room temperature and then washed 4 times with 1×TBST. The detection antibody, goat anti-mouse IgG (Fc)-HRP (Jackson Immunoresearch, cat #115-036-008) was diluted 1:5,000 in 1× TBST and added to the ELISA plates. Plates were then sealed and incubated in the dark for 1 hour at room temperature. After washing the plates 4 times with 1×TBST, TMB substrate (SeraCare, cat #5120-0076) was added to each well and incubated in dark for 15 minutes at room temperature. Reactions were then stopped with 1N sulfuric acid and plates read on the Biotek ELx800 plate reader. Concentrations of gp96 expressed from each sample were then determined based off the standard curve.

The amount of mouse OX40L protein expressed by the mHS-130 cells was also determined by ELISA. For each sample to be tested, one million B16F10-Ova9 parental and mHS-130 cells were plated in a 6-well tissue culture plate in a total volume of 1 ml each. Cells were incubated at 37° C. with 5% CO₂ for 24 hours at which point the supernatants were harvested. Supernatants were then centrifuged at 2500 rpm for 5 minutes to pellet any cell debris. Clarified supernatants were then transferred to new 1.5 ml tubes and stored at −80° C. Samples collected were from freshly thawed vials of cells.

To perform the ELISA, 96-well plates were coated with 2.5 μg/ml His-tagged mouse OX40 protein (Acro Biosystems, cat #OXO-M5228) in PBS. Plates were sealed and stored at 4° C. overnight. Plates were then washed 4 times with 1×TBST and then blocked with 1% BSA (Sigma-Aldrich, cat #A2153) for 1 hour at room temperature. The plates were then washed 4 times with 1×TBST and a mouse IgG1-mouse OX40L standard (Thermo Fisher Scientific, lot #2214217) was prepared in IMDM with 10% FBS. A 2000 ng/ml mlgG1-mOX40L standard solution was made and 2-fold serial dilutions were performed down to 1.95 ng/ml. Sample supernatants were loaded onto the ELISA plates and 2-fold serial dilutions were performed. Plates were sealed and incubated for 90 minutes at 37° C. and then washed 4 times with 1×TBST. The detection antibody, goat anti-mouse IgG (Fc)-HRP (Jackson Immunoresearch, cat #115-036-008), was diluted 1:5000 in 1×PBS/0.05% Tween 20/0.1% BSA and added to the ELISA plates. Plates were then sealed and incubated in the dark for 1 hour at room temperature. After washing the plates 4 times with 1×TBST, TMB substrate was added to each well and incubated in dark for 10 minutes at room temperature. Reactions were then stopped with 1 N sulfuric acid and plates read on the Biotek ELx800 plate reader. Concentrations of OX40L expressed from each sample were then determined based off the standard curve. This protocol is based on human OX40L protocol.

TABLE 3
Expression of mouse gp96-Ig and OX40L-Ig from mHS-110 and
mHS-130, respectively, per million cells in a 24-hour period
Mouse gp96-Ig (nanograms/mL per million cells in 24 hrs)

| | | 1^{st} | 2^{nd} | 3^{rd} | |
|---|---|---|---|---|---|
| | Prime Dose: | Boost Dose: | Boost Dose: | Boost Dose: | Average |
| Replicate | Concentration | Concentration | Concentration | Concentration | Expression |
| 1 | 99.27 | 122.93 | 124.92 | 79.06 | 106.55 |
| 2 | 106.47 | 136.21 | 139.55 | 86.22 | 117.11 |
| 3 | 101.89 | 113.04 | 136.21 | 96.66 | 111.95 |
| 4 | 102.05 | 126.87 | 130.78 | 82.32 | 110.51 |
| 5 | 100.74 | 135.98 | 147.70 | 81.00 | 116.36 |
| 6 | 109.90 | 120.35 | 139.89 | 87.60 | 114.44 |
| Mean | 103.39 | 125.90 | 136.51 | 85.47 | 112.82 |
| STDEV | 4.00 | 9.10 | 7.91 | 6.35 | 6.24 |
| CV % | 3.87 | 7.23 | 5.80 | 7.42 | — |

Mouse OX40L-Ig (nanograms/mL per million cells in 24 hrs)

| | | 1^{st} | 2 ^{nd} | 3^{rd} | |
|---|---|---|---|---|---|
| | Prime Dose: | Boost Dose: | Boost Dose: | Boost Dose: | Average |
| Replicate | Concentration | Concentration | Concentration | Concentration | Expression |
| 1 | 400.97 | 513.57 | 424.8 | 413.8 | 413.19 |
| 2 | 372.13 | 455.03 | 460.6 | 411.4 | 414.71 |
| 3 | 314.70 | 456.81 | 415.1 | 405.3 | 378.37 |
| 4 | 453.23 | 572.00 | 535.9 | 484.0 | 491.04 |
| 5 | 408.47 | 517.90 | 538.0 | 447.1 | 464.52 |
| 6 | 374.08 | 530.53 | 474.0 | 440.6 | 429.56 |
| Mean | 387.3 | 484.3 | 474.7 | 433.7 | 445.00 |
| STDEV | 46.2 | 41.3 | 52.9 | 29.9 | 42.58 |
| CV % | 11.92 | 11.49 | 11.1 | 6.9 | — |

TABLE 4
Expression level of active biologic protein per
cell type and resulting ratio for each group

| Group | GP96 ng | OX40L ng | Ratio (dose level) | Animals |
|---|---|---|---|---|
| A | 38 | 50 | 1:1.3 (low) | 5 |
| B | 113 | 147 | 1:1.3 (med) | 5 |
| C | 339 | 441 | 1:1.3 (high) | 5 |
| D | 38 | 95 | 1:2.5 (low) | 5 |
| E | 113 | 283 | 1:2.5 (med) | 5 |
| F | 339 | 848 | 1:2.5 (high) | 5 |
| G | 38 | 190 | 1:5 (low) | 5 |
| H | 113 | 565 | 1:5 (med) | 5 |
| I | 339 | 1695 | 1:5 (high) | 5 |
| J | 38 | N/A | N/A | 5 |

Mice were tail bled consecutively on days 3, 5, 7, 10, 12, and 14 post-immunization and days 17, 19, 21, 24, 26, 28, 33, 38, 41, 45, 48, and 54 into heparinized PBS (10 units/ml) and lysed using ACK lysis buffer (150 mM NH₄Cl, 100 mM KHCO₃ and 10 mM EDTA 0.2 Na, pH 7.2) for 3 minutes and neutralized with 1×PBS. Samples from the OT-1 transfer experiments were then centrifuged at 300×g for 5 minutes, supernatant was removed and the cell pellet stained with anti-CD3 (20 pg/ml), anti-CD44 (20 μg/ml), anti-CD127 (20 μg/ml), anti-KLRG1 (20 μg/ml) and anti-CD8 (5 μg/ml) antibody cocktail made in FACS buffer using Alexa Fluor 700 anti-mouse CD3 (BioLegend cat #100216), PE-Cy7 anti-mouse CD44 antibody (Biolegend Cat #103030), PE anti-mouse CD127 (BioLegend Cat #135010), MBL TRP2 tetramer (MBL cat #T03014B, H-2K^{b} TRP2 “SVYDFFVWL” (SEQ ID NO:40)) (used forspleen only), APC anti-mouse KLRG1 (BioLegend Cat #138412) and Brilliant Violet 421 anti-mouse CD8alpha antibody (BioLegend Cat #100738) for 30 minutes at 4° C. Consecutive bleeds were collected from the peripheral blood and analysis was performed by flow cytometry on both exogenous, adoptively transferred, OT-1 and endogenous CD8+ and CD4+ T-cells for activation, and short (SLECs) and long-term (MPECs) memory markers as outlined in the methods section. Tumor growth kinetics, response rates and infiltrating lymphocytes, were also quantitated.

Intracellular Cytokine Staining and Flow Cytometry: Splenocytes (1×10⁶) were incubated with synthetic peptides; SIINFEKL, gp100, TRP-1, TRP-1-Variant, or TRP-2 in the wells of a 96-well plate at 37° C. and 5% CO2. Synthetic peptides were added to a final concentration of 0.5 μM with Golgi stop and incubated for 4-10 hours depending on the peptide. Plates were spun, medium was removed, and cells were resuspended with surface markers CD8 and CD3 and incubated at 4° C. for 20 minutes. Cells were washed, resuspended in 50 μl of BD Cytofix/Cytoperm, and incubated at 4° C. for 20 min before another two washes and staining with anti-IFN-γ. Cells were washed once before acquisition and analysis of fluorescence. Analysis was done using FlowJo software (Tree Star Inc.); events were gated for live lymphocytes on FSC×SSC followed by CD8+ cells using CD8× CD3 and displayed as CD8×IFN-γ.

B16F10-OVA tumor challenge and volume calculations: Melanoma B16F10 cells were harvested and resuspended at a concentration of 5×10⁵ cells/100 μl in a volume of 80 μl HBSS and 20 μl Matrigel. C57BL/6 mice were subcutaneously injected with 100 μl of B16F10 cells (5×10⁵ cells/mouse) on the inner abdomen 29 days post-OT-1 transfer and 28 days post-primary vaccination, designated “day 28”, as shown in the study design (FIG. 13). The tumor size was measured and documented every 3 days with a caliper, starting on day 7, and calculated using the formula (A×B; A as the largest and B as the smallest diameter of tumor). Tumor growth was documented as standard error mean. To record the survival of the tumor-bearing mice, either natural death or a tumor volume greater than 450 mm² leading to death was counted as death. Each experimental group included five animals.

Tumor tissue digestion for Tumor Infiltrating Lymphocytes (TILs): The MACS Miltenyl Biotec tumor dissociation kit was used for this procedure (cat #130096-730).

ELISPOT Assay: Splenocytes were harvested and red blood cell lysis buffer (cat #36858500, Roche) was used to eliminate red blood cells. Cells were washed in IMDM medium and pelleted. Counted cells were resuspended in IMDM with 10% FBS. Each ELISPOT well received 1 million cells, in a total volume of 200 μl. Treatments included the use of B16F10-OVA parental line lysates (vial of 10 million cells; freeze-thawed three times) at a 10-fold dilution, immunodominant epitopes for B16F10 tumors: “gp100/pmel” (EGSRNQDWL (SEQ ID NO:38)), “TRP-1/gp75” (TWHRYHLL (SEQ ID NO:39)), TRP-2 (SVYDFFVWL (SEQ ID NO:40)) and the MHC-I restricted peptide for H2b haplotype for OVA (SIINFEKL (SEQ ID NO:41)). All peptides were used at a final concentration of 10 μg/mL. PMA/lonomycin was used as a positive control at “1 to 400 dilution” 100 nM of PMA, 1.6 μM ionomycin (500× stock from eBioscience, cat #00-4970-03, and whole chicken ovalbumin protein “OVA” at 250 μg/ml.

Interferon-gamma capture ELISPOT assay was carried out as previously published (Klinman et al., Current Protocols in Immunology. 1994 June (1): 6.19.1-6.19.8). Briefly, MAIP N45 Milipore 96-well filtration plates were coated with anti-mouse IFN-γ purified monoclonal antibody (clone AN18) in PBS saline at 10 μg/mL, 50 μl/well, at 4° C., overnight. The next day, cells were washed in IMEM medium plus 10% FBS at 200 ul/well, washed three times, then blocked for 1 hour at 25° C. Cells were processed as described above and 1 million or 100,000 splenocytes were added to each well and treated per the description above with various peptide stimulations. Each plate was incubated in a 37° C., 5% CO₂, non-vibrating incubator for 16 hrs or overnight. At the end of the incubation, cells were flicked into the sink to stop the culture and washed with PBS+0.1% Tween-20 three times and blotted hard onto paper towels to get rid of residual liquid after the last wash. Secondary biotinylated antibody was added at 50 μl per well, 4 μg/mL of anti-IFN-γ (clone AN18), incubated at 25° C. for 2 hrs. Plates were then washed again with PBS+0.1% Tween-20 three times. Peroxidase-conjugated streptavidin from Jackson research labs was then added at a 1 to 1000 dilution from stock concentration in PBS+0.1% Tween-20+2% BSA, 50 μl per well, 30-minute incubation at 4° C. Plates were then washed with PBS+0.1% Tween three times. The plastic backing of the ELISPOT plate was removed and the entire plate was submerged and soaked in PBS+0.1% Tween-20 for 1 hour at 25° C. The plate was then washed in PBS to remove the tween, then 100 μl of the AEC substrate (Vector kit) added in 100 mM of TRIS buffer at pH 8.2 was added to each well to allow for development of spots. At the end of the incubation, 10-20 minutes, all plates incubated for the same time, the reaction as stopped by rinsing the plates with tap water and air dried.

Plates were scanned and counted in an AID Autoimmun Diagnostika GMBH iSpot monochromatic ELISPOT reader (2018). Counting parameters for each plate remained consistent across all plates that were developed together as to prevent bias, as spot development, density and magnitude is influence by the operator, reagent lots and final substrate development time.

### Experimental Results

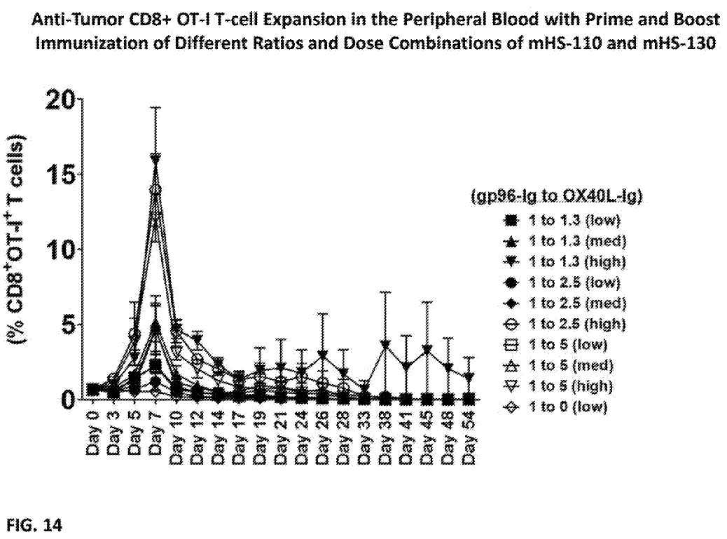

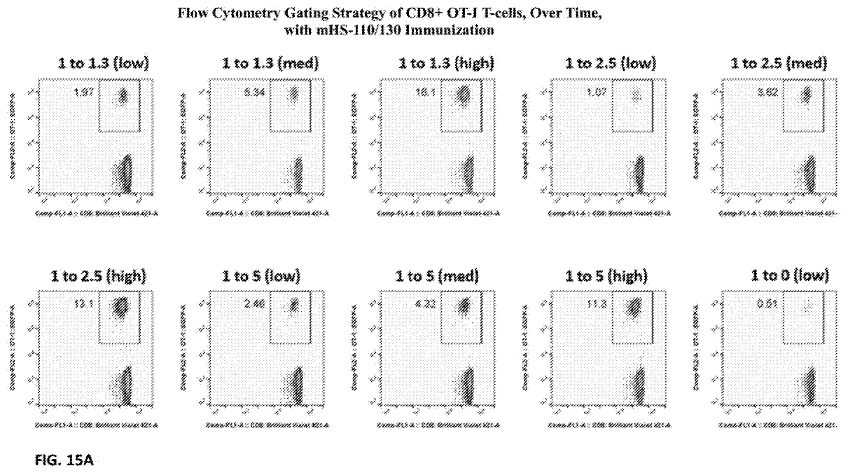

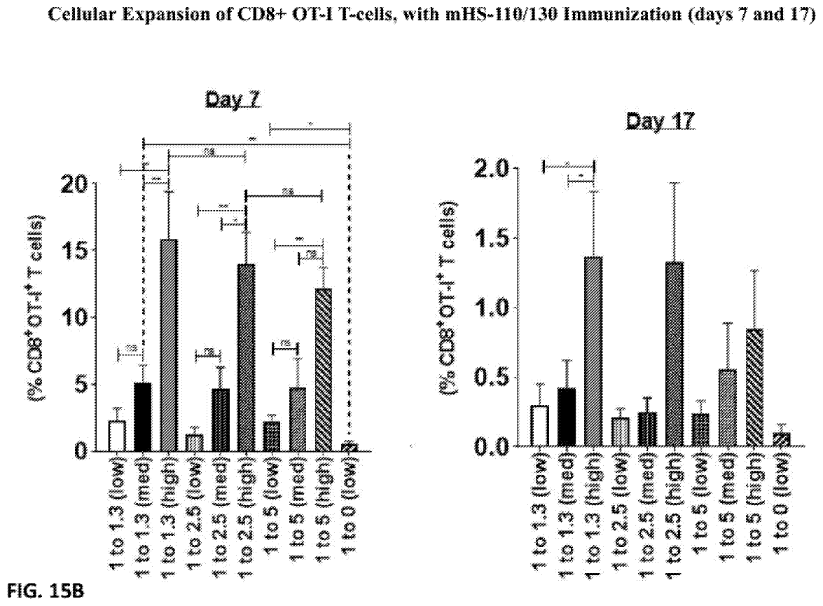

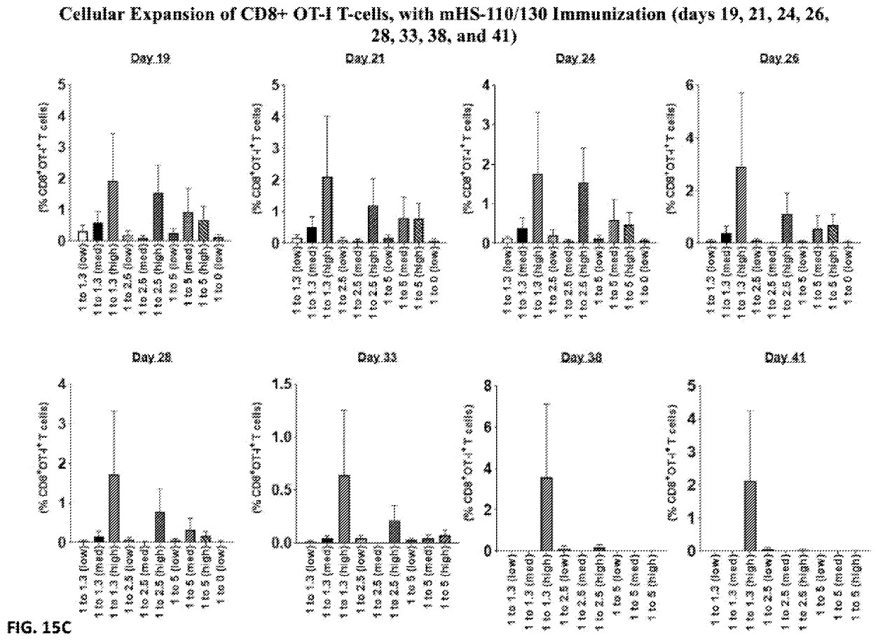

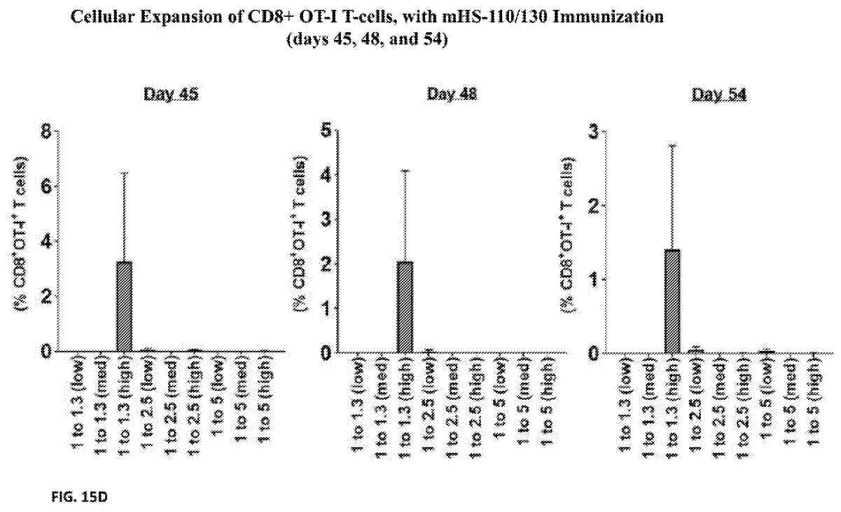

As shown in FIGS. 14, 15A, 15B, 15C, and 15D, priming with mHS-110 (gp96-Ig) and mHS-130 (OX40L-Ig) produced a primary immune response and cellular expansion of OT-1 cells (anti-OVA, CD8+ TCR transgenic T-cells) in a dose and ratio dependent manner.

FIG. 14 illustrates anti-tumor CD8+OT-I T cell expansion in the peripheral blood with prime and boost Immunization of different ratios and dose combinations of mHS-110 and mHS-130. Recipient mice were injected with mHS-110 and mHS-130 at different ratios and doses of gp96-Ig to OX40L-Ig. OT-I GFP+CD8+ T cells were analyzed in the blood on days 0-54 days post-vaccination. Mice were boosted on day 14 with the same ratios of mHS-110 and mHS-130 as in the primary phase, and OT-I GFP+CD8+ T cells were analyzed in the blood days post-challenge. As shown, the ratio 1:1.3 ratio of mHS-110 to mHS-130 (high dose) results in the peak of CD8+OT-I T cell expansion at day 7 and remains higher than other dose ratios through day 54.

FIG. 15A illustrates gating strategy for flow cytometry experiments of the study of FIG. 13. Recipient mice were injected with a mHS-110 and mHS-130 at different ratios and doses of gp96-Ig to OX40L-Ig. OT-I GFP+CD8+ T cells were analyzed in the blood on days 0-54 days post-vaccination. Mice were boosted on day 14 and 31 with the same ratios of mHS-110 and mHS-130 as in the primary phase, and OT-I GFP+CD8+ T cells were analyzed in the blood days post-challenge. Tumor was provided on day 28.

FIG. 15B, illustrating expansion of OT-1 cells on days 7 and 17, shows that the dose ratio 1 to 1.3 provided the best response with the greatest expansion seen for the high dose, 339 ng gp96 to 441 ng OX40L. In particular, upon the primary immunization, there was a dramatic, nearly a three-fold increase in OT-1 cells after adding OX40L to gp96 (38 ng gp96 vs. 50 ng group; for the 1 to 1.3 ratio) when compared to mHS-110 (38 ng gp96) alone, as shown in FIG. 15B. There was a trend decrease in expansion of OT-1 cells that indirectly correlated with an increase in OX40L-Ig to gp96-Ig, as shown for day 7 in FIG. 15B. This trend was observed for other days (from day 7 to day 26) but is most pronounced at the primary immunization peak of day 7. Boosting on day 14 produced an increase in cellular expansion, and, among the studied dose ratio groups, the observed expansion was not pronounced for the 1 to 1.3 ratio (FIG. 15B, day 17). The groups that harbored the most OT-1 cells just prior to the boosting on day 14, showed the best and most rapid response shown on day 17 through day 26, with the same dose trend between the ratios and groups, especially as it correlates to OX40L-Ig, as shown in FIG. 15C. The 1 to 1.3 ratio of gp96-Ig to OX40L-Ig maintained the best expansion of CD8+OT-I T-cells through the end of the study, as shown in FIG. 15D illustrating the results for days 45, 48, and 54.

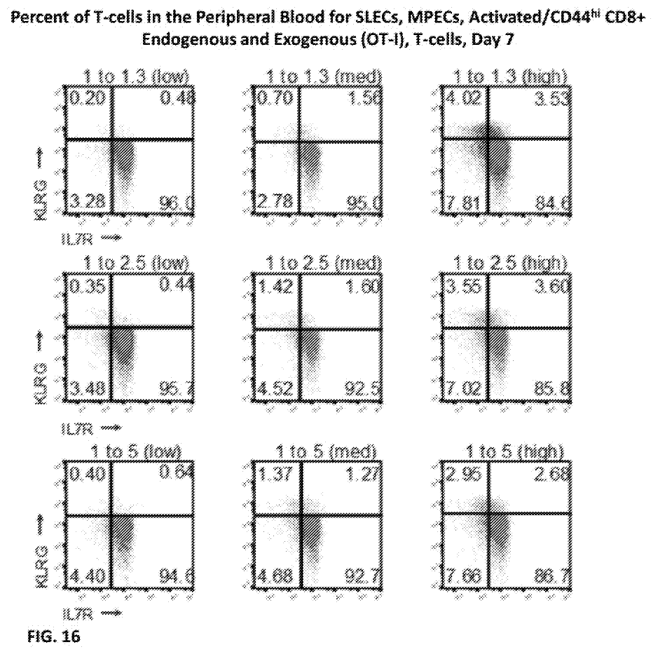

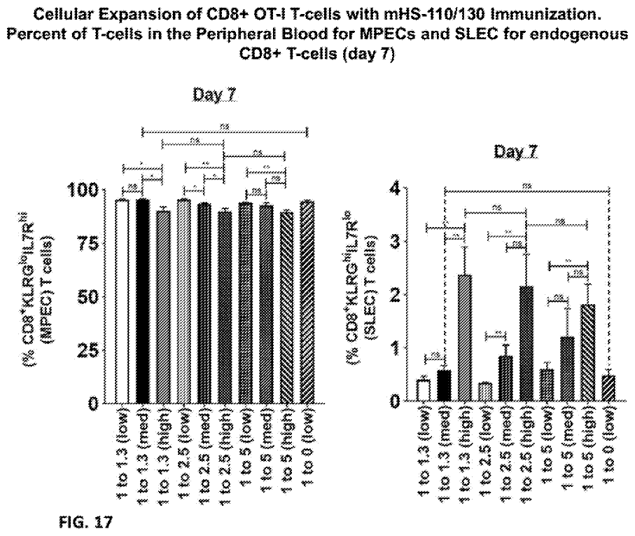

In the present study, activation and key memory markers were also measured for the studied peripheral blood populations. Flow cytometry gating strategy for memory markers KLRG1 and IL-7R is shown in FIG. 16. Further, as shown in FIG. 17, significant changes in CD8+ memory precursor effector cells (MPECs) (KLRG1^{lo} IL-7R^{hi}) and short-lived effector cells (SLECs) (KLRG^{hi} IL-7R^{lo}) were observed for endogenous cell populations on day 7. Short-lived effector cells increased directly proportional to gp96-Ig exposure; but indirectly proportional to OX40L-Ig (FIG. 17). This suggests that increasing OX40L may produce better CD8+ T-cell expansion. These data support an approach of combining both gp96 and OX40L in a single vaccine.

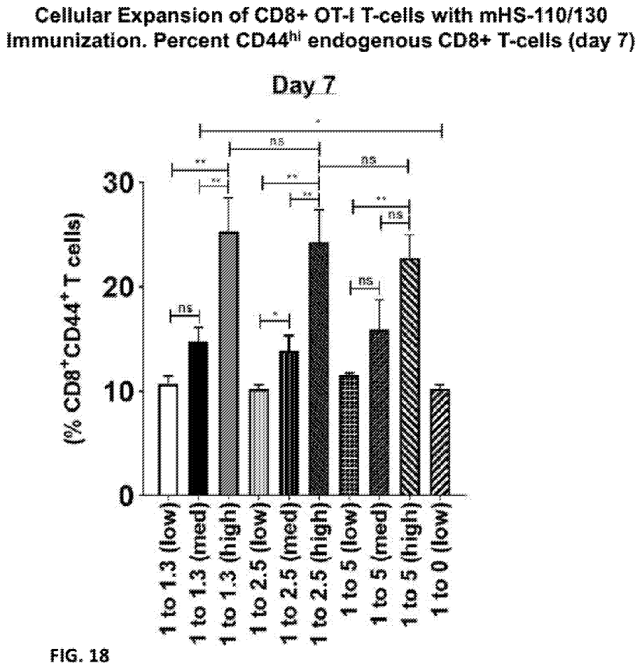

In addition, similar to what was observed with OT-1 T-cell expansion in FIGS. 15B-15D, greater OX40L to less gp96 stimulation results in a more robust cellular response, resulting in greater SLEC formation (FIG. 17, day 7, endogenous CD8+ T-cells). Formation of SLECs correlated with increased activation, as shown by the upregulation of adhesion molecule, CD44, on endogenous CD8+ T-cell populations (FIG. 18, day 7). Expression of CD44, which appears on antigen stimulated T-cells to allow entry to target tissues, is highly elevated on endogenous populations and trends with the increased dose levels of OX40L relative to gp96.

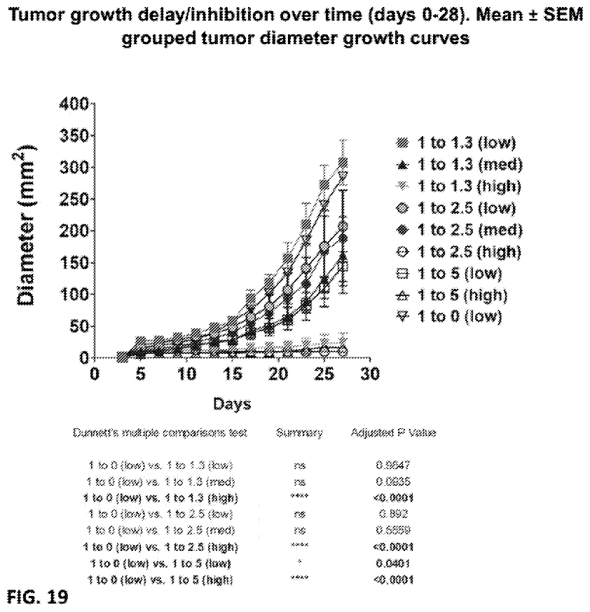

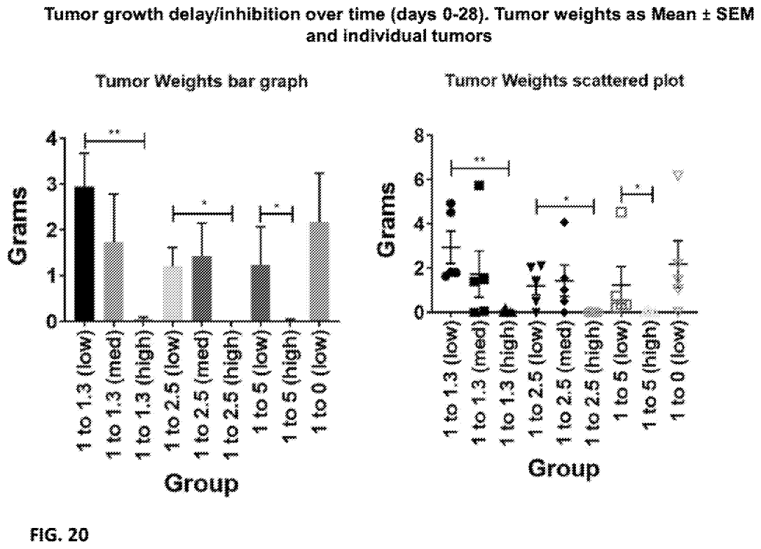

On day 28, B16F10-OVA tumors were given subcutaneously, and tumor delay challenge began. Tumor growth inhibition for each group tested is shown in FIG. 19. Significant differences, as compared to mHS-110 (38 ng gp96-Ig) alone, measured by 1-way ANOVA statistical test were observed for the 339 ng to 441 ng group (1 to 1.3, high, gp96 to OX40L), 339 ng to 848 ng (1 to 2.5, high, gp96 to OX40L), and 339 ng to 1695 ng (1 to 5, high, gp96 to OX40L) (FIG. 19, ****p<0.0001). End of study (day 55) tumor weights, grouped (FIG. 20, left) and individual (FIG. 20, right) agree with the caliper measurements (FIG. 19).

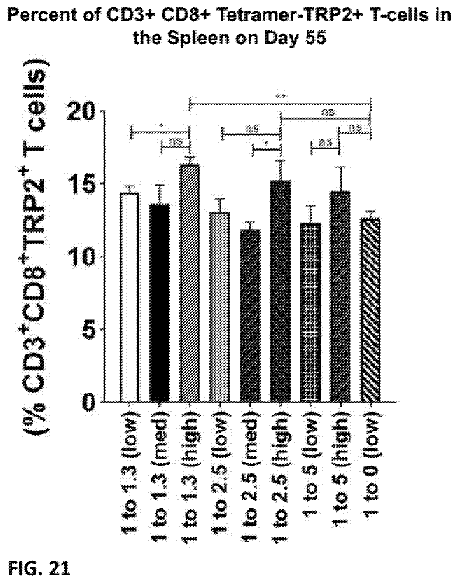

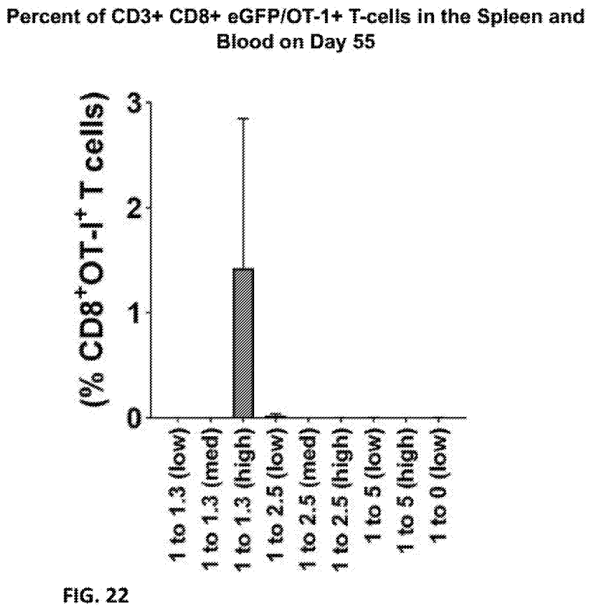

Tumor-specific, TRP2 tetramer positive CD8+ T-cells increased with treatment in a dose-dependent manner and showed a significant increase over treatment with 38 ng of gp96-Ig via mHS-110 alone (FIG. 21) As for the transferred CD8+OT-1+eGFP cells, at day 55, only the 1 to 1.3 ratio of 339 ng gp96-Ig to 441 ng OX40L-Ig dose (high dose) showed any presence of expanded subsets on day 55 in both the spleen and blood, as shown in FIG. 22.

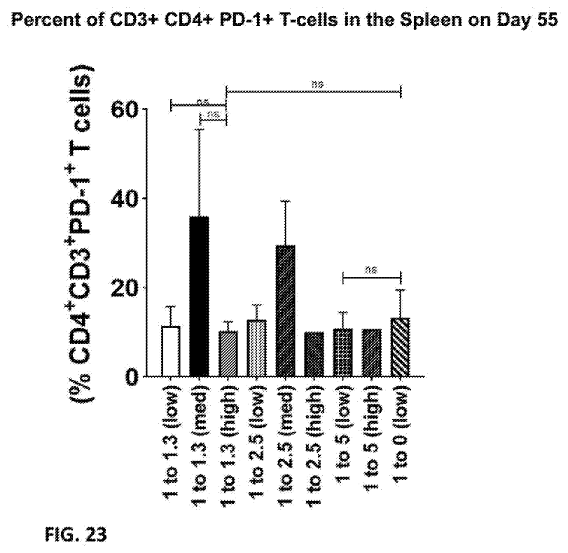

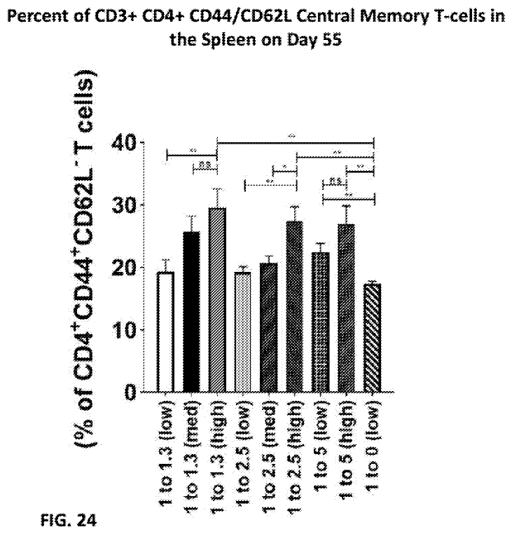

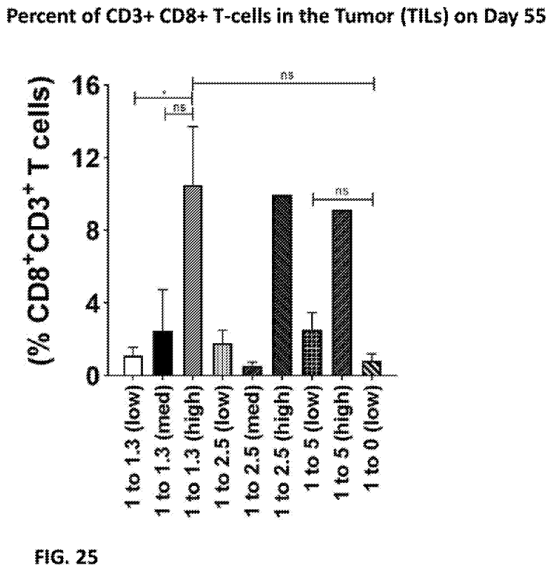

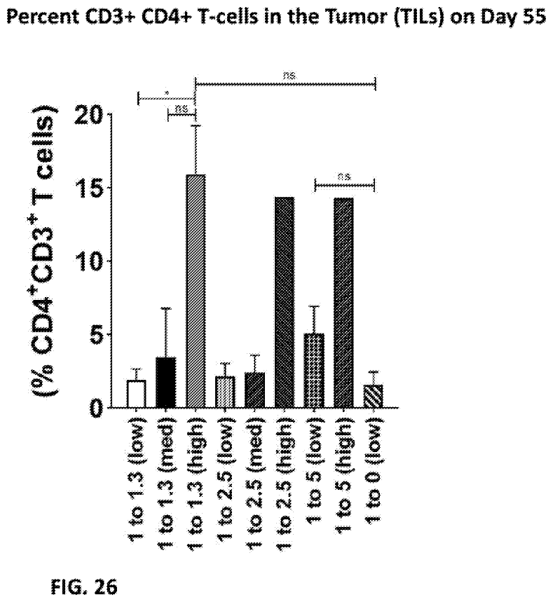

FIG. 23 illustrates an increased percentage of exhausted CD4+ T-cells staining positive for PD-1 in the spleen (% of CD3+CD4+PD-1+ T cells) on day 55. The percentage of effector memory CD4+ T-cells in the spleen of treated and tumor-burden mice changed with vaccination, in an OX40L dose-dependent manner, as shown in FIG. 24. This increase in the percentage of activated CD4+ T-cells correlated with an increase proportion of CD4+ T-cell infiltrating the tumor (TILs), and was dose dependent (see FIG. 26).

The present study demonstrates that 1) both CD4+ and CD8+ T-cell subsets are expanded with gp96/OX40L-Ig co-vaccinations that correlate with increased TIL percentages and tumor growth inhibition; 2) the best dose combination for long-term survival and expansion of tumor specific CD8+ T-cells cells is the 1 to 1.3 ratio at a dose of 339 ng of gp96-Ig to 441 ng of OX40L-Ig; and 3) a dose determines tumor growth inhibition, with 38 ng of gp96-Ig to 50 ng of OX40L-Ig being the no-observed effect level (NOEL) and 113 ng gp96-Ig to 147 ng of OX40L-Ig being the minimum active biological effect level (MABEL) for this dose combination, in mice.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

### INCORPORATION BY REFERENCE

All patents and publications referenced herein are hereby incorporated by reference in their entireties. The publications discussed herein are provided solely for their disclosure prior to the filing date of the present application. Nothing herein is to be construed as an admission that the present invention is not entitled to antedate such publication by virtue of prior invention. As used herein, all headings are simply for organization and are not intended to limit the disclosure in anyway.

1.-2. (canceled)

3. A method for treating cancer in a patient in need thereof, comprising administering to the patient an effective amount of:
(a) a first cell comprising an expression vector comprising a nucleotide sequence that encodes a secretable vaccine protein and
(b) a second cell comprising an expression vector comprising a nucleotide sequence that encodes a T cell costimulatory fusion protein and wherein the T cell costimulatory fusion protein enhances activation of antigen-specific T cells when administered to the subject.

4. The method of claim 3, wherein the secretable vaccine protein is a secretable gp96-Ig fusion protein.

5. The method of claim 4, wherein the Ig tag in the gp96-Ig fusion protein comprises the Fc region of human IgG1, IgG2, IgG3, IgG4, IgM, IgA, or IgE.

6. The method of claim 3, wherein the T cell costimulatory fusion protein is selected from OX40L-Ig, or a portion thereof that binds to OX40: ICOSL-Ig, or a portion thereof that binds to ICOS; 4-1BBL-Ig, or a portion thereof that binds to 4-1BBR; TL1A-Ig, or a portion thereof that binds to TNFRSF25: GITRL-Ig, or a portion thereof that binds to GITR; CD40L-Ig, or a portion thereof that binds to CD40; and CD70-Ig, or a portion thereof that binds to CD27.

7.-12. (canceled)

13. The method of claim 6, wherein the Ig tag in the T cell costimulatory fusion protein comprises the Fc region of human IgG1, IgG2, IgG3, IgG4, IgM, IgA, or IgE.

14. The method of claim 3, wherein the expression vector is incorporated into a virus or virus-like particle.

15. The method of claim 3, wherein the expression vector is incorporated into a human tumor cell.

16. The method of claim 3, wherein the patient is a human cancer patient.

17. The method of claim 16, wherein administration to the human patient increases the activation or proliferation of tumor antigen specific T cells in the patient.

18. The method of claim 17, wherein the activation or proliferation of tumor antigen specific T cells in the patient is increased by at least 25 percent as compared to the level of activation or proliferation of tumor antigen specific T cells in the patient prior to the administration.

19. The method of claim 18, comprising administering in combination with an agent that inhibits immunosuppressive molecules produced by tumor cells.

20. The method of claim 19, wherein the agent is an antibody against PD-1.

21. The method of claim 20, wherein the antibody against PD-1 is selected from nivolumab, pembrolizumab, pidilizumab, cemiplimab, AGEN2034, AMP-224, AMP-514, PDR001.

22.-24. (canceled)

25. The method of claim 3, wherein the T cell costimulatory molecule enhances the activation of antigen-specific T cells in the subject to a greater level than gp96-Ig administration alone.

26. The method of claim 3, wherein the ratio of the secretable vaccine protein to the T cell costimulatory fusion protein is about 1:1.

27. The method claim 3, wherein the ratio of the secretable vaccine protein to the T cell costimulatory fusion protein is about 1:1.3.

28. The method claim 3, wherein the ratio of the secretable vaccine protein to the T cell costimulatory fusion protein is about 1:10.

29. The method of claim 4, wherein the secretable gp96-Ig fusion protein lacks the gp96 KDEL (SEQ ID NO:3) sequence.

30. The method of claim 15, wherein the human tumor cell is a lung adenocarcinoma cell line.
